(19) United States
(12) Patent Application Publication
(10) Pub. No.: US 2026/0091028 A1
(43) Pub. Date: Apr. 2, 2026

(54) METHOD OF TREATING AMYOTROPHIC LATERAL SCLEROSIS WITH PRIDOPIDINE

(71) Applicant: PRILENIA NEUROTHERAPEUTICS LTD., Yakum (IL)

(72) Inventors: Michael HAYDEN, Yakum (IL); Michal GEVA, Even-Yehuda (IL)

(73) Assignee: PRILENIA NEUROTHERAPEUTICS LTD., Yakum (IL)

(21) Appl. No.: 19/391,284

(22) Filed: Nov. 17, 2025

Related U.S. Application Data
(63) Continuation-in-part of application No. 18/416,210, filed on Jan. 18, 2024.
Application No. 18/416,210 is a continuation-in-part of application No. 18/172,927, filed on Feb. 22, 2023.
Application No. 18/172,927 is a continuation-in-part of application No. 17/076,069, filed on Oct. 21, 2020, now abandoned.
Application No. 17/076,069 is a continuation-in-part of application No. 16/789,564, filed on Feb. 13, 2020, now Pat. No. 11,406,625.
Application No. 16/789,564 is a continuation-in-part of application No. PCT/US2018/046481, filed on Aug. 13, 2018.
(60) Provisional application No. 62/545,315, filed on Aug. 14, 2017.

(51) Int. Cl. A61K 31/451; A61K 9/48; A61K 31/192; A61K 31/4152; A61K 31/4245; A61K 31/428; A61K 31/439; A61K 31/485; A61K 31/575; A61K 31/661; A61K 31/7016; A61K 33/242; A61P 25/28

(52) U.S. Cl. CPC A61K 31/451; A61K 9/4825; A61K 31/192; A61K 31/4152; A61K 31/4245; A61K 31/428; A61K 31/439; A61K 31/485; A61K 31/575; A61K 31/661; A61K 31/7016; A61K 33/242; A61P 25/28

(57) ABSTRACT

Provided herein is a method for treating a human subject afflicted with ALS by administering to the subject a therapeutically effective amount of pridopidine or pharmaceutically acceptable salt thereof.

### CROSS REFERENCE TO RELATED APPLICATIONS

This application is a Continuation-in-Part from U.S. application Ser. No. 18/416,210 filed Jan. 18, 2024, which is a Continuation-in-Part from U.S. application Ser. No. 18/172,927 filed Feb. 22, 2023, which is a Continuation-in-Part from U.S. application Ser. No. 17/076,069 filed Oct. 21, 2020, which is a Continuation-in-Part application from U.S. application Ser. No. 16/789,564 filed Feb. 13, 2020, which is a Continuation-in-Part application from International Patent Application No. PCT/US2018/046481 filed Aug. 13, 2018, which claims the benefit of U.S. Provisional Application No. 62/545,315, filed Aug. 14, 2017, the entire content of which are incorporated herein by reference.

### BACKGROUND OF THE INVENTION

### Amyotrophic Lateral Sclerosis

Amyotrophic lateral sclerosis (ALS) is a devastating degenerative disease characterized by progressive loss of motor neurons in the motor cortex, brainstem, and spinal cord (Peters 2015). This rapidly progressing fatal disease leads to weakness of limb, respiratory, and bulbar muscles. Patients progressively lose control of voluntary muscles, leading to loss of limb function and the ability to chew, swallow, speak and eventually breathe.

ALS is a rare condition, having a mean incidence rate of 2.8/100,000 in Europe and 1.8/100,000 in North America, and a mean prevalence rate of 5.40/100,000 in Europe and 3.40/100,000 in North America (Bozzoni 2016).

About 10% of ALS cases are classified as familial (fALS), whereas the remaining 90% are classified as sporadic (sALS) and occur randomly (Riva 2016). Over 60% of patients die within 3 years of presentation, usually from respiratory failure and about 10% survive for more than 10 years (Zou 2016). There are currently three approved drugs for the treatment of ALS, all of which confer a modest effect on disease progression and survival. These include riluzole, edaravone and AMX0035. Nuedexta is approved only for treating pseudobulbar effect (symptomatic) in ALS patients.

Clinical manifestations of ALS include muscle weakness and hypotrophy, fasciculations and cramps, spastic hypertonus, and hyperreflexia are the main clinical manifestations. Some patients also display dysarthria, dysphagia, and respiratory weakness. Non-motor symptoms include behavioral disturbances, dysexecutive impairment, and frontotemporal dementia.

The neuropathological features of ALS include muscle atrophy, loss of anterior horn cells, and sclerosis of the spinal cord lateral columns (Martel 2016). Gliosis, defined as activation of astrocytes and microglia, is also a hallmark of ALS.

### Pridopidine

The chemical name of pridopidine is 4-(3-(Methylsulfonyl)phenyl)-1-propylpiperidine, and its Chemical Registry Number is CAS 346688-38-8 (CSID: 7971505, 2016). The Chemical Registry number of pridopidine hydrochloride is 882737-42-0 (CSID: 25948790 2016). Processes of synthesis of pridopidine and a pharmaceutically acceptable salt thereof are disclosed in U.S. Pat. No. 7,923,459 and PCT Application Publication No. WO 2017/015609. U.S. Pat. No. RE46,117 discloses pridopidine for the treatment of a variety of diseases and disorders.

Pridopidine is a high affinity and highly selective S1R ligand which has ˜30-fold higher affinity towards the S1R vs D3Rs, and ˜500-fold higher affinity vs D2Rs. Selective binding of pridopidine for the S1R with no dopamine D2/D3R binding was confirmed using positron emission tomography (PET) imaging in rats (Sahlholm, 2015), and in humans (TV7820-IMG-10082). The neuroprotective properties of pridopidine are demonstrated in preclinical models of ALS and other neurodegenerative diseases and are mediated by its activation of the S1R, as its silencing by genetic or pharmacological methods abolishes the protective effects of pridopidine.

The S1R is a highly conserved transmembrane protein located in the endoplasmic reticulum (ER) and specifically enriched in the subregions contacting mitochondria (Mitochondria-Associated Membranes, MAM). The S1R is highly enriched in the CNS. The S1R is a key component of the ER-Mitochondria axis, and is thus implicated in cellular differentiation, neuroplasticity, neuroprotection, and cognitive function in the brain.

### SUMMARY OF THE INVENTION

The present invention is based, at least in part, on the surprising experimental discovery that pridopidine treatment improves axonal transport deficits, enhances ERK activation and restores neuromuscular junction (NMJ) activity in SOD1 impaired muscle cell co-cultures, reduces mutant SOD1 aggregates in the spinal cord, and attenuates NMJ disruption and subsequent muscle wasting in SOD1 impaired mice.

The invention is also based on the results of a clinical trial in which the effect of pridopidine or pharmaceutically acceptable salt thereof was assessed in ALS subjects.

The invention provides a method for treating amyotrophic lateral sclerosis (ALS) in a subject, comprising administering to the subject an effective amount of pridopidine or pharmaceutically acceptable salt thereof.

In some embodiments provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof. In other embodiments, the subject has faster disease progression as measured by the ALSFRS-R pre-baseline slope. In another embodiment the subject has faster disease progression as measured by the baseline NfL levels. In other embodiments, the subject has early ALS with less than 18 months from symptom onset. In other embodiments, the subject has faster disease progression as measured by the ALSFRS-R pre-baseline slope and early with <18 months from symptom onset.

In other embodiments, the symptom is impairment in muscle strength.

In some embodiments, the symptom is impairment in speech. In other embodiments, the impairment of speech comprises reduced speaking rate, reduced phonation time, reduced articulation rate and reduced articulation precision.

In some embodiments, the symptom is impairment in functionality. In other embodiment, the impairment in functionality comprises speech, salivation, swallowing, handwriting, cutting food and handling utensils, dressing and hygiene, turning in bed and adjusting bed clothes, walking, climbing stairs, dyspnea, orthopnea, respiratory insufficiency or any combination thereof.

In some embodiments, the symptom is impairment in respiratory function. In other embodiments, the respiratory function is assessed by slow vital capacity (SVC) or forced vital capacity (FVC) or by the ALSFRS-R-Respiratory sub-domain.

In some embodiments, the symptom is impairment in bulbar function. In other embodiments, the bulbar function is measured by the ALSFRS-R bulbar subdomain (Q1-Q3) score. In other embodiments, the bulbar function is measured by the CNS-BFS. In other embodiments, the bulbar function comprises of impaired speech, swallowing or salivation.

In some embodiments provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof, wherein the amount of pridopidine or pharmaceutically acceptable salt thereof is effective in maintaining, reducing or lessening the increase in neurofilament light (NfL) protein levels in a human subject afflicted with ALS.

In some embodiments provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof, wherein the amount of pridopidine or pharmaceutically acceptable salt thereof is administered daily, twice a week, three times a week or more often than once daily. In other embodiments the amount of pridopidine or pharmaceutically acceptable salt thereof is administered orally. In other embodiments, the amount of pridopidine or pharmaceutically acceptable salt thereof administered is 10 mg per day to 90 mg per day. In other embodiments, the pridopidine salt is pridopidine hydrochloride.

In some embodiments provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof and further comprising administering to the subject a second composition comprising a therapeutically effective amount of a Second compound, wherein the Second compound is riluzole, edaravone, dextromethorphan/quinidine, sodium phenylbutyrate (PB), tauroursodeoxycholic acid, sodium phenylbutyrate (PB)/tauroursodeoxycholic acid, SLS-005 (Trehalose), DNL343, CNM-Au8 nanocrystalline gold or ABBV-CLS-7262. In other embodiments, the second compound precedes the administration of pridopidine or pharmaceutically acceptable salt thereof. In other embodiments, the administration of pridopidine or pharmaceutically acceptable salt thereof precedes the administration of the Second compound. In some embodiments, the pridopidine or pharmaceutically acceptable salt thereof is administered adjunctively to the Second compound. In other embodiments, the Second compound is administered adjunctively to the pridopidine or pharmaceutically acceptable salt thereof.

In some embodiments provided herein, the method further comprises evaluating treatment efficacy using speech- and communication-related endpoints. In certain embodiments, the assessment includes quantitative measures of speech intelligibility obtained through in-clinic or remote recordings.

### BRIEF DESCRIPTION OF THE FIGURES

FIGS. 1A-1C: Axonal transport assay. FIG. 1A. Experimental workflow for the axonal transport assay. Approximately 5 days after plating in the soma compartment, motor neuron axons cross into the axonal compartment. On day 6, pridopidine is added to both compartments for overnight incubation. On day 7, fluorescent QDot-BDNF is added to the axonal compartment and imaged using a confocal microscope in live cells. FIG. 1B. Schematic illustration of the experimental system for axonal transport tracking in motor neurons (MNs), showing the locations of the proximal (soma) and distal compartments, with the axonal compartment connecting them. Qdot-BDNF is added to the distal compartment, and retrogradely transported to the cell body in the soma compartment. FIG. 1C. Time lapse images and kymograph of Qdot-BDNF (marked with arrow) axonal transport. A kymograph plots movement over time (y axis) on a straight line, i.e. an axon (x axis).

FIGS. 2A-2B: Graphs showing effect of pridopidine on instantaneous velocity values and particle stop count of Qdot-BDNF along the axon (WT=wild type) FIG. 2A. Pridopidine increases axonal transport which is impaired in SOD1G93A ALS neurons in a S1R-mediated mechanism. Pridopidine's effect on instantaneous velocity values (μm/sec) for Qdot-BDNF particles in WT, SOD1G93A or Sigma-1 receptor knock out (S1R−/−) MNs. Instantaneous velocity is reduced ˜10% in SOD1 neurons. Pridopidine treatment significantly increases particle velocity by ˜25% and ˜35% at the 0.1 and 1 μM doses, respectively. Riluzole (Rilu.), the standard of care for ALS, has no effect on particle velocity. The effect of pridopidine is abolished in cells in S1R−/− cells, indicating that this effect is mediated by the S1R. FIG. 2B. Pridopidine increases axonal transport which is impaired in SOD1G93A ALS neurons in a S1R-mediated mechanism: stop counts. Pridopidine's effect on particle stop count (number of counted stops of Qdot-BDNF per second). Pridopidine (1 μM) significantly reduces the number of stop counts ˜2.5-fold compared to untreated SOD1 neurons (p<0.01). Pridopidine's effect is abolished in S1R−/− neurons (p<0.001). Riluzole has no effect on particle stop counts. Data are shown as the mean±SEM. *p value <0.05; **p value <0.01; ***p value <0.001 (n=6 independent experiments; the sample size for each experiment is indicated on bars; Student's t test).

FIG. 3: Schematic illustration of the experimental procedure for neuromuscular coculture assays measuring muscle innervation and Neuro Muscular Junction function (NMJ). Spinal cord explant is cultured in the proximal compartment and primary myocytes are cultured in the distal compartment. Pridopidine is added to both proximal and distal compartments.

FIG. 4: Pridopidine increases axonal growth which is impaired in SOD1G93A neurons. The number of grooves in which axons cross to the muscular compartment is diminished ˜10-fold in SOD1 neurons (p<0.05). Pridopidine increases the number of grooves containing axons ˜3-fold (p<0.05).

FIGS. 5A-5B: Recreation of function neuromuscular junctions in an in vitro model in a microfluidic chamber (MFC). FIG. 5A. Microscope image of neuromuscular junction in microfluidic co-culture chamber. Upper panel: Phase image of a myocyte in the distal compartment connected by axons (arrowheads). Lower panel: High magnification images of myocyte: MN contact points. The muscle compartment is visualized by fluorescently marking the acetyl choline receptor (AchR) with fluorescently-labelled bungarotoxin (Btx). The neuronal compartment is marked by expressing green fluorescent protein (GFP) on the MN-specific genetic marker HB9. (Hb9:GFP) Inset: rendering of colocalization. FIG. 5B. Muscle contraction traces as extracted from intensity over time measurements of muscle contraction in microfluidic co-culture chamber. A contracting myofiber demonstrates increased intensity peaks.

FIG. 6: Pridopidine increases the innervation rate of myocytes by MN axons. Graph showing pridopidine's effect on axonal innervation rate in WT and SOD1G93A (SOD1) ALS myocytes. SOD1 axons demonstrate a 50% reduction in innervation rate compared to WT axons. Pridopidine increases innervation rate in SOD1 axons 2-fold, back to levels comparable to WT.

FIG. 7: Pridopidine increases the percent of contracting myocytes in a S1R-dependent manner. SOD1 G93A myocytes show reduced contractility compared to WT cells (p<0.05). SOD1 myocytes innervated with S1R−/− neurons show a bigger decrease in contractility (p<0.0001). Pridopidine increases the percent of contracting myocytes in WT cells (p<0.05), and restores the percent of contracting myocytes in SOD1 G93A muscles in a S1R-mediated mechanism (p<0.001). In cultures in which the S1R has been knocked out (S1R-KO), pridopidine's effect is abolished. *p value <0.05; **p value <0.01, ***p value <0.001, ****p value <0.0001. (n=number of microfluidic chambers from 3 or more independent experiments; Student's t test)

FIGS. 8A-8B: Pridopidine increases phosphorylated ERK levels in a S1R-mediated mechanism. FIG. 8A. Representative images of Western blot analysis of phosphorylated and total (extracellular-signal-regulated kinase) ERK levels in motor neuron extracts from WT, SOD1 and S1R−/− cultures. Pridopidine increases pERK in a dose-dependent and S1R-dependent manner. Effect of pridopidine on ERK levels. FIG. 8B. Quantification of pridopidine's effect on ERK activation as measured by pERK (phosphorylated ERK levels). Pridopidine increases pERK levels in both WT and SOD1G93A cells, with optimal effect at 1 μM. Data are shown as the mean pERK/tERK±SEM. *p value <0.05, ˜p value <0.1 (n=3 independent experiments; Student's t test.)

FIGS. 9A-9C: Pridopidine reduces mutant SOD1 aggregation in the spinal cord of SOD1 mice. Effect of pridopidine on mutant SOD1 aggregates. FIG. 9A. Visualization and quantification of fluorescently labeled spinal cords with NC500 to label mutant SOD1 aggregates in WT and SOD1G93A (ALS) spinal cords, from mice treated or not with pridopidine 30 mg/kg. FIG. 9B. Pridopidine reduces mutant SOD1 aggregation in the spinal cord of SOD1 mice-quantification in gray matter (GM). Quantitative analysis of the number of SOD1 aggregates per area identified in the gray matter. In SOD1 mice, aggregates are increased ˜7-fold compared to WT mice (p<0.01). Pridopidine treatment reduces aggregates by ˜50% (p<0.05). FIG. 9C Pridopidine reduces mutant SOD1 aggregation in the spinal cord of SOD1 mice—quantification in white matter (WM). Quantitative analysis of the number of SOD1 aggregates per area identified in the white matter. SOD1 increases the number of NSC-positive aggregates by more than 7-fold (p<0.01). Pridopidine treatment reduces aggregation by ˜60% (p<0.01). Data are shown as the mean±SEM. *p value <0.05; **p value <0.01 (n=4 mice in each group; one-way ANOVA followed by Fisher's LSD post hoc tests.).

FIGS. 10A-10B: Pridopidine prevents muscle fiber wasting in SOD1 mice. FIG. 10A. Representative images of hematoxylin and eosin (H&E)-stained cross sections from Gastrocnemius muscle of WT or SOD1G93A mice treated or not with Pridopidine 30 mg/kg. Pridopidine rescues muscle fiber wasting in SOD1 G93A muscles. FIG. 10B. Assessment of pridopidine's effect on muscle fiber wasting: quantitative analysis of pridopidine's effect on muscle fiber diameter. Muscle fiber diameter is reduced in SOD1 mice compared to WT (p<0.001). Pridopidine rescues muscle fiber wasting in SOD1 G93A muscles (p<0.05). Data are shown as mean±SEM (n=number of NMJs). *p value <0.05; **p value <0.01; ***p value <0.001 (n=5 mice in each group; Student's t test).

FIGS. 11A-11B: Pridopidine preserves neuromuscular junctions in SOD1 mice.

FIG. 11A. Representative images of immunostained neuromuscular junctions in gastrocnemius muscle of SOD1 mice. The muscle compartment is marked by fluorescently labelled bungarotoxin (Btx). The neuronal compartment is marked by immunostaining of neurofilament heavy (NFH) and synaptophysin (SynP) proteins. FIG. 11B. Pridopidine preserves neuromuscular junctions in SOD1 mice (quantification). In SOD1 mice, the number of innervated NMJs is reduced by ˜60% (p<0.01). Pridopidine treatment rescues NMJ innervation back to ˜80% of WT (p<0.05). Pridopidine prevents loss of NMJs in SOD1 G93A muscles. Quantitative analysis of pridopidine's effect on the percentage of innervated NMJs. Data are shown as mean±SEM (n=number of NMJs). *p value <0.05; **p value <0.01; ***p value <0.001 (n=5 mice in each group; Student's t test).

In the following FIGS. 12-20, the effect of pridopidine, compound 1 and compound 4 was evaluated individually in primary motor neurons (MNs) from the Transactive Response DNA binding protein (TDP)43 lacking the nuclear localization signal (ΔNLS) mouse model of ALS. The TDP43 ΔNLS model is a doxycycline (DOX)-inducible model. With DOX, motor neurons are healthy. Upon DOX withdrawal, TDP43 without the nuclear localization signal (ΔNLS) accumulates in the cytoplasm, creating toxic TDP43 aggregates that are a hallmark of ALS, and recapitulating the downstream neurotoxic effects seen in ALS. In the figures below, neurite health was evaluated by (1) cell body cluster area; (2) cell body cluster count, and (3) neurite length at two timepoints: 7 days in vitro (DIV) and 14 DIV using the Incucyte automated imaging system. DOX withdrawal in FIGS. 12-20 results in a significant reduction of 20-40% in all of these parameters. Data is mean±SEM, n=5 with 5 technical repeats per experiment (indicated by dots on the graph). One-way ANOVA test. P-values: *p<0.05, **p<0.01, ***p<0.001, ****p<0.0001).

In FIGS. 12A-12B, 13A-13B and 14A-14B, the effect of pridopidine on all these parameters was evaluated.

FIGS. 12A-12B: Pridopidine rescues cell body cluster area in TDP43 ΔNLS neurons. FIG. 12A. Pridopidine rescues cell body cluster area in TDP43 ΔNLS motor neurons, 7 DIV. DOX withdrawal results in a significant ±40% reduction (p<0.0001) in cell body cluster area. Pridopidine increases cell body cluster area at 0.01, 20, 30, and 80 nM (p<0.05), as well as at 0.05, 1, 10, and 50 nM (p<0.01). The greatest effect is observed at the low concentrations of 0.01 and 0.05 nM.

FIG. 12B. Pridopidine rescues cell body cluster area in TDP43 ΔNLS neurons, 14 DIV. DOX withdrawal results in a significant ±20% reduction (p<0.01) in cell body cluster area. Pridopidine increases cell body cluster are back to WT levels at 0.05, 10, 20, 30 and 80 nM. (p<0.05) as well as at 1 nM (p<0.01) and 50 nM (p<0.001).

FIGS. 13A-13B: Pridopidine rescues cell body cluster count in TDP43 ΔNLS neurons. FIG. 13A: Pridopidine rescues cell body cluster count in TDP43 ΔNLS neurons, 7 DIV. DOX withdrawal results in a significant #15% (p<0.01) reduction cell body cluster count. Pridopidine increases cell body cluster count at all concentrations tested, significantly at 0.01 and 5 nM (p<0.05), 0.05, 1, 20 nM (p<0.01), 10, 30, 50, 60 and 80 nM (p<0.001). FIG. 13B: Pridopidine rescues cell body cluster count in TDP43 ΔNLS neurons, 14 DIV. DOX withdrawal results in a significant ±20% (p<0.01) reduction in cell body cluster count. Pridopidine at all concentrations increases cell body cluster count back to control levels, significantly at 10, 20 and 80 nM (p<0.05) and 0.05, 1 and 50 nM (p<0.01).

FIG. 14A-14B: Pridopidine rescues neurite length in TDP43 ΔNLS neurons. FIG. 14A: Pridopidine rescues neurite length in TDP43 ΔNLS neurons, 7 DIV. DOX withdrawal results in a significant ±25% (p<0.01) reduction in neurite length. Pridopidine at all concentrations tested increases neurite length back to control levels. The effect is most significant at concentrations of 10, 20, 30, 50, 80 and 100 nM (p<0.05). FIG. 14B: Pridopidine rescues neurite length in TDP43 ΔNLS neurons, 14 DIV. DOX withdrawal results in a significant ±40% reduction (p<0.0001) in neurite length. Pridopidine increases neurite length at all concentrations tested. The effect is largest and most significant at the lowest concentrations of 0.01 and 0.05 nM (p<0.01). It is also significant at concentrations of 20, 30 and 80 nM (p<0.05) and 1, 10, and 50 nM (p<0.01).

In FIGS. 15A-15B, 16A-16B, and 17A-17B, the effect of compound 1 on all parameters was evaluated.

FIG. 15A-15B: Compound 1 rescues cell body cluster area in TDP43 ΔNLS neurons. FIG. 15A: Compound 1 rescues cell body cluster area in TDP43 ΔNLS neurons, 7 DIV. DOX withdrawal results in a significant ±20% decrease (p<0.05) in cell body cluster area. Compound 1 increases cell body cluster area at all concentrations tested. The effect is largest and most significant at 10, 25, 50, 100 and 500 nM concentrations (p<0.0001). A significant effect is also observed at 1 nM (p<0.05) and 0.001 and 0.01 nM (p<0.01) as well as at 75 nM (p<0.001). FIG. 15B: Compound 1 rescues cell body cluster area in TDP43 ΔNLS neurons, 14 DIV. DOX withdrawal results in a significant #30% reduction (p≤xxx) in cell body cluster area. Compound 1 increases cell body cluster area at all concentrations tested: 5 nM (p<0.001) and 1, 10, 25, 50, 75, 100 and 500 nM (p<0.0001)

FIG. 16A-16B: Compound 1 rescues cell body cluster count in TDP43 ΔNLS neurons. FIG. 16A: Compound 1 rescues cell body cluster count in TDP43 ΔNLS neurons, 7 DIV. DOX withdrawal results in a significant #15% reduction in cell body cluster count (p<0.0001). Compound 1 increases cell body cluster count at all concentrations tested. The effect is largest and most significant in concentrations 1, 10, 25, 50, 75, 100 and −500 nM (p<0.0001). A significant effect is also observed at 5 nM (p<0.001). FIG. 16B: Compound 1 rescues cell body cluster count in TDP43 ΔNLS neurons, 14 DIV. DOX withdrawal results in a significant ±25% reduction in cell body cluster count (p<0.01). Compound 1 at all concentrations tested induced an increase in cell body cluster count. The effect is largest and most significant at 1, 10, 50, 100 and 500 nM concentrations (p<0.0001). A significant increase is also observed at 5 nM (p<0.01) and 25 and 75 nM (p<0.001).

FIG. 17A-17B: Compound 1 rescues neurite length in TDP43 ΔNLS neurons. FIG. 17A: Compound 1 rescues neurite length in TDP43 ΔNLS neurons, 7 DIV. DOX withdrawal results in a significant ±30% reduction in neurite length (p<0.0001). Compound 1 increases neurite length back to control levels at all concentrations tested. Significant effects are observed at 0.01 nM (p<0.05), 0.001 nM (p<0.01), 1 μM (p<0.001) and 1, 5, 10, 25, 50, 75, 100 and 500 nM (p<0.0001). FIG. 17B: Compound 1 rescues neurite length in TDP43 ΔNLS neurons, 14 DIV. DOX withdrawal results in a significant ±30% reduction in neurite length (p<0.0001). Compound 1 at increases neurite length at all doses. A significant effect is observed at 0.01 nM (p<0.01), 0.001 and 5 nM (p<0.001) and 1, 10, 25, 50, 75, 100 and 500 nM (p<0.0001).

In FIGS. 18A-18B, 19A-19B, and 20A-20B, the effect of compound 4 on all parameters was evaluated.

FIG. 18A-18B: Compound 4 rescues cell body cluster area in TDP43 ΔNLS neurons. FIG. 18A: Compound 4 rescues cell body cluster area in TDP43 ΔNLS neurons, 7 DIV. DOX withdrawal results in a significant ±25% reduction in cell body cluster area (p<0.05). Compound 4 increases cluster area at all concentrations tested. Significant effects are observed at 0.01 and 5 nM (p<0.05), 0.1 nM, 1 nM and 1 μM (p<0.01) and most significantly at 10, 25, 50, 75, 100 and 500 nM (p<0.0001) FIG. 18B: Compound 4 rescues cell body cluster area in TDP43 ΔNLS neurons, 14 DIV. DOX withdrawal results in a significant ±25% reduction in cell body cluster area (p<0.01). Compound 4 increases cell body cluster area at all concentrations tested. A significant effect is observed at 1 nM (p<0.05), 1 μM (p<0.01), 25 and 100 nM (p<0.001) and 10, 50, 75 and 500 nM (p<0.0001)

FIG. 19A-19B: Compound 4 rescues cell body cluster count in TDP43 ΔNLS neurons. FIG. 19A: Compound 4 rescues cell body cluster count in TDP43 ΔNLS neurons, 7 DIV. DOX withdrawal results in a significant ±20% decrease in cell body cluster count (p<0.05). Compound 4 increases cell body cluster count at all concentrations tested. The effect is significant at concentrations 1 nM, 5 nM and 1 μM (p<0.05), as well as at 10, 25, 50, 75, 100 and 500 nM (p<0.0001). FIG. 19B: Compound 4 rescues cell body cluster count in TDP43 ΔNLS neurons, 14 DIV. DOX withdrawal causes a significant ±30% reduction in cell body cluster count (p<0.001). All concentrations tested of compound 4 increase cell body cluster count. A significant effect is observed at 25 nM and 1 μM (p<0.01), 10, 100 and 500 nM (p<0.001) and 50 and 75 nM (p<0.0001)

FIG. 20A-20B: Compound 4 rescues neurite length in TDP43 ΔNLS neurons. FIG. 20A: Compound 4 rescues neurite length in TDP43 ΔNLS neurons, 7 DIV. DOX withdrawal results in a significant (p<0.01)+20% decrease in neurite length. Treatment with compound 4 increases neurite length significantly at all concentrations tested: 0.01 nM (p<0.05), 0, 0.1 nM (p<0.01), 1 nM, 5 nM and 1 μM (p<0.001) and 10, 25, 50, 75, 100 and 500 nM (p<0.0001) FIG. 20B: Compound 4 rescues neurite length in TDP43 ΔNLS neurons, 14 DIV. DOX withdrawal results in a significant ±25% decrease in neurite length (p<0.0001). Compound 4 treatment significantly increases neurite length at 0.1 and 1 nM (p<0.01), 0.01 nM (p<0.001) and 10, 25, 50 75, 100 and 500 nM as well as at 1 μM).

In the following brief descriptions of the figures and the corresponding figures, the efficacy of pridopidine for treating subjects afflicted with ALS was assessed as part of the HEALEY ALS Platform trial detailed in Experiment 9. Efficacy measures were collected throughout the 24-week double-blind period and analyzed as change from baseline vs. placebo. Rate of change (change/month) was analyzed using the Random-Slopes model. Additional analysis was done using the Mixed Models for Repeated Measures (MMRM) analysis. For subgroup analysis, subjects were classified by time from symptom onset (<18 months was defined as a cutoff), faster progressors (by pre-baseline ALSFRS-R slope >=0.75 and >=1.0) and El Escorial criteria for ALS diagnosis.

FIG. 21. Change from baseline pridopidine vs. placebo in ALSFRS-R Total score at week 24. Pridopidine demonstrates improvement vs placebo in ALSFRS-R Total score in the full analysis set (FAS) of subjects (change vs. placebo 0.26, p=0.67). The effect is larger in subjects who are faster progressors with an ALSFRS-R pre-baseline slope ≥0.75 (change vs, placebo 1.08, p=0.35), and early subjects who have <18 months from symptom onset (change vs., placebo 1.41, p=0.17), The effect of pridopidine on ALSFRS-R Total is strongest in subjects with definite ALS diagnosis (per El Escorial criteria) who are early with <18 months from symptom onset (change vs, placebo 2.4, p=0.19). MMRM statistical model, positive change indicates improvement.

FIG. 22: Change from baseline pridopidine vs. placebo in ALSFRS-R Total score at week 24, in definite+probable ALS subjects. Pridopidine demonstrates improvement vs. placebo in ALSFRS-R Total in subjects with definite+probable ALS (per El Escorial definition) (change vs, placebo 1.24, p=0.07). This effect is larger and statistically significant in definite+probable subjects who are early in the disease with <18 months from symptom onset (change vs, placebo 2.9, p=0.03). A stronger improvement is observed in definite+probable subjects who are faster progressors with pre-baseline slope ≥1 (change vs. placebo 3.4, p=0.07). The largest, statistically significant improvement is seen in definite+probable subjects who are early <18 months from symptom onset and faster progressors with pre-baseline slope ≥1 (change vs. placebo 5.2, p=0.04). MMRM model; positive change indicates improvement.

FIGS. 23A-23C: change from baseline in ALSFRS-R Total Score per visit. Graphs illustrate the change from baseline in ALSFRS-R Total Score at 8, 16 and 24 weeks. Pridopidine shows less decline in ALSFRS-R Total score vs placebo over time. Negative change indicates worsening. FIG. 23A: Change from baseline in all subjects (FAS) who are faster progressors with pre-baseline ALSFRS-R slope ≥0.75. FIG. 23B: Change from baseline in all subjects (FAS) who are early with <18 months from symptom onset. FIG. 23C: Change from baseline in definite ALS subjects who are early with <18 months from symptom onset.

FIG. 24A-24B: change from baseline in ALSFRS-R Total Score per visit. Graphs illustrate the change from baseline in ALSFRS-R Total Score at 8, 16 and 24 weeks. Pridopidine shows less decline in ALSFRS-R Total score vs placebo over time. Negative change indicates worsening. FIG. 24A: change from baseline in full analysis set (FAS) subjects who are early with <18 months from symptom onset and faster with pre-baseline slope ≥1, per visit. Pridopidine demonstrates a significant less decline vs. placebo in ALSFRS-R Total Score at week 8 (change from baseline pridopidine −1.81 vs. placebo −4.63, p=0.043) and at week 16 (change from baseline pridopidine −4.68 vs. placebo −9.15, p=0.03). The strong trend towards improvement is maintained at 24 weeks (change vs. placebo 4.19, p=0.07). FIG. 24B: change from baseline in definite+probable subjects who are early with <18 months from symptom onset and faster with pre-baseline slope ≥1, per visit. Pridopidine demonstrates a significant less decline vs. placebo in ALSFRS-R Total Score at week 8 (change from baseline pridopidine −1.94 vs. placebo −5.42, p=0.02) week 16 (change from baseline pridopidine −4.97 vs. placebo −10.41, p=0.014), and week 24 (change from baseline pridopidine −7.51 vs. placebo −12.71, change vs. placebo −12.71, p=0.04).

FIG. 25: Change from baseline vs. placebo in ALSFRS-R Respiratory Score to 24 weeks, Random Slope Model. Pridopidine demonstrates a trend towards improvement vs placebo in the full analysis set (FAS, change vs placebo 0.09, p=0.06). The effect is larger in subjects with pre-baseline slope ≥0.75 (change vs. placebo 0.11, p=0.26) and in subjects who are early with <18 months from symptom onset (change vs. placebo 0.11, p=0.14). The largest effect is seen in definite ALS subjects who are early with <18 months from symptom onset (change vs placebo 0.2, p=0.12). Positive change indicates improvement.

FIG. 26. Change from baseline vs. placebo in ALSFRS-R Respiratory Scale to 24 weeks, MMRM Model. Pridopidine demonstrates improvement in the full analysis set of subjects (FAS, change vs placebo 0.44, p=0.09). The effect is larger in subjects who are faster progressors with pre-baseline slope ³ 0.75 (change vs. placebo 0.53, p=0.34) and in subjects who are early with <18 months from symptom onset (change vs. placebo 0.79, p=0.08). The largest effect is seen in definite ALS subjects who are early with <18 months from symptom onset (change vs placebo 1.04, p=0.18). Positive change indicates improvement.

FIGS. 27A-27D: Change from baseline in ALSFRS-R Respiratory Score per visit, MMRM model. Graphs illustrate the change from baseline in ALSFRS-R Respiratory Score at 8, 16 and 24 weeks. Pridopidine shows less decline vs placebo in ALSFRS-R Respiratory score over time. Negative change indicates worsening. FIG. 27A: Change from baseline in ALSFRS-R Respiratory Score per visit, Full analysis set (FAS). Pridopidine demonstrates less decline vs placebo in ALSFRS-R Respiratory Score vs. placebo at 8, 16 and 24 weeks (change from baseline pridopidine −0.79 vs. placebo −1.24, p=0.09 at 24 weeks). FIG. 27B: Change from baseline in ALSFRS-R Respiratory Score, FAS & slope ³ 0.75. Pridopidine demonstrates less decline vs. placebo 16 and 24 weeks (change from baseline pridopidine −1.13 vs. placebo −1.66, at 24 weeks p=0.34). FIG. 27C: Change from baseline in ALSFRS-R Respiratory Score per visit, FAS & symptom onset <18 months. Pridopidine demonstrates less decline vs. placebo in subjects who are early with <18 months from symptom onset at weeks 8, 16 and 24 (change from baseline at week 24 pridopidine −0.83 vs. placebo −1.61, p=0.08). FIG. 27D: Change from baseline in ALSFRS-R Respiratory Score per visit, in definite ALS diagnosis in subjects who are early with, symptom onset <18 months. Pridopidine demonstrates less decline vs. placebo in definite ALS subjects with <18 months from symptom onset at weeks 16 and 24 (change from baseline at 24 weeks pridopidine −0.94 vs. placebo −1.98, p=0.18).

FIG. 28: Change from baseline pridopidine vs. placebo in ALSFRS-R Respiratory Scale to 24 weeks, in definite+probable ALS subjects, MMRM Model. Pridopidine demonstrates significant less decline in definite+probable ALS subjects (change vs placebo 0.73, p=0.02). The effect is larger in subjects <18 months from symptom onset (change vs. placebo 1.2, p=0.03). A beneficial effect is observed in subjects with pre-baseline slope ³ 1 (change vs. placebo 1.11, p=0.21). The largest beneficial effect of pridopidine is seen in definite+probable ALS subjects who are early with <18 months from symptom onset and faster with pre-baseline slope ³ 1 (change vs placebo 1.81, p=0.08). Positive change indicates improvement.

FIGS. 29A-29D: Change from baseline in ALSFRS-R Respiratory Score per visit, definite+probable ALS subjects, MMRM model. Graphs illustrate the change from baseline in ALSFRS-R Respiratory Score at 8, 16 and 24 weeks. Pridopidine shows less decline vs placebo in ALSFRS-R Respiratory score over time. Negative change indicates worsening. FIG. 29A: Change from baseline in ALSFRS-R Respiratory Score per visit, definite+probable ALS. Pridopidine demonstrates a significant less decline in ALSFRS-R Respiratory Score vs. placebo at 8, 16 and 24 weeks (change from baseline at week 24 pridopidine −0.71 vs. placebo −1.45, p=0.02). FIG. 29B: Change from baseline in ALSFRS-R Respiratory Score per visit, definite+probable ALS, slope ≥1. Pridopidine demonstrates less decline vs. placebo in subjects with pre-baseline slope ≥1 at 8, 16 and 24 weeks (change from baseline at week 24 pridopidine-1.3 vs. placebo −2.4, p=0.21). FIG. 29C: Change from baseline in ALSFRS-R Respiratory Score per visit, definite+probable ALS, symptom onset <18 months. Pridopidine demonstrates less decline vs. placebo in subjects with <18 months from symptom onset at weeks 8, 16 and 24 (change from baseline pridopidine −0.74 vs. placebo −1.94, p=0.03). FIG. 29D: Change from baseline in ALSFRS-R Respiratory Score, per visit, definite+probable ALS, symptom onset <18 months and pre-baseline slope ≥1. Pridopidine demonstrates less decline vs. placebo in definite+probable ALS subjects with <18 months from symptom onset and pre-baseline slope ≥1 at weeks 8, 16 and 24 (change from baseline at week 24 pridopidine −1.05 vs. placebo −2.86, p=0.08).

FIG. 30: Change from baseline pridopidine vs. placebo in ALSFRS-R Bulbar Score to 24 weeks, Full analysis set (FAS), MMRM Model. Pridopidine demonstrates less decline vs placebo in the full analysis set of subjects (FAS, change vs placebo 0.11, p=0.6). The effect is larger in subjects who are faster progressors with pre-baseline slope ≥0.75 (change vs. placebo 0.14, p=0.75) and in subjects who are early with <18 months from symptom onset (change vs. placebo 0.32, p=0.4). The largest effect is seen in definite ALS subjects who are early <18 months from symptom onset (change vs placebo 0.82, p=0.23). Positive change indicates improvement.

FIGS. 31A-31B: Change from baseline in ALSFRS-R Bulbar Score per visit. Graphs illustrate the change from baseline in ALSFRS-R Bulbar Score at 8, 16 and 24 weeks. Pridopidine mitigates the decline in ALSFRS-R Bulbar score over time. Negative change indicates worsening. FIG. 31A: Change from baseline in ALSFRS-R Bulbar Score per visit, Full analysis set (FAS)<18 months from symptom onset. Pridopidine demonstrates less decline in ALSFRS-R Bulbar Score vs. placebo at 16 and 24 weeks (change from baseline at week 24 pridopidine −1.18 vs. placebo −1.49, p=0.4). FIG. 31B: Change from baseline in ALSFRS-R Bulbar Score per visit, definite ALS subjects <18 months from symptom onset. Pridopidine demonstrates less decline vs. placebo at 16 and 24 weeks (change from baseline at week 24 pridopidine −1.53 vs. placebo −2.53, p=0.23).

FIG. 32: Change from baseline pridopidine vs. placebo in ALSFRS-R Bulbar Score to 24 weeks, definite+probable ALS, MMRM Model. Pridopidine demonstrates less decline vs placebo in definite+probable subjects (change vs placebo 0.36, p=0.19). The effect is larger in definite+probable subjects who are early with <18 months from symptom onset (change vs. placebo 0.93, p=0.059) and in definite+probable subjects who are faster with pre-baseline slope ≥1 (change vs. placebo 0.41, p=0.57). A large effect is also seen in definite+probable ALS subjects who are early with <18 months from symptom onset and faster with pre-baseline slope ≥1 (change vs placebo 0.81, p=0.33). Positive change indicates improvement.

FIG. 33: Change from baseline pridopidine vs. placebo in Speaking Rate (syllables/second) to 24 weeks, MMRM Model. Pridopidine demonstrates a significant improvement in the full analysis set of subjects (FAS, change vs placebo 0.2, p=0.009). The effect is larger in subjects who are early with <18 months from symptom onset (change vs. placebo 0.31, p=0.009). The effect is largest and most significant in subjects who are faster progressors with pre-baseline slope ≥0.75 (change vs. placebo 0.53, p=0.0005). A similarly large effect is seen in definite ALS subjects who are early with <18 months from symptom onset (change vs placebo 0.53, p=0.02). Positive change indicates improvement.

FIG. 34: Change from baseline pridopidine vs. placebo in Speaking rate (syllables/second) to 24 weeks, definite+probable ALS, MMRM Model. Pridopidine demonstrates a significant improvement in definite+probable subjects (change vs placebo 0.35, p=0.0001). The effect is larger in subjects who are early with <18 months from symptom onset (change vs. placebo 0.43, p=0.004) and larger in subjects who are faster progressors with pre-baseline slope ≥1 (change vs. placebo 0.95, p=0.00001). The largest, significant effect seen in definite ALS subjects who are early with <18 months from symptom onset and faster with pre-baseline slope ≥1 (change vs placebo 1.08, p=0.00003). Positive change indicates improvement.

FIG. 35: Change from baseline pridopidine vs. placebo in Articulation Rate (syllables/second) to 24 weeks, MMRM Model. Pridopidine demonstrates a significant improvement in the full analysis set of subjects (FAS, change vs placebo 0.15, p=0.048). The effect is larger and more significant in subjects who are early with <18 months from symptom onset (change vs. placebo 0.28, p=0.009). The effect is largest and most significant in subjects with who are faster progressors with a pre-baseline slope ≥0.75 (change vs. placebo 0.57, p=0.00002). A similarly large and statistically significant effect is seen in definite ALS subjects who are early with <18 months from symptom onset (change vs placebo 0.48, p=0.013). Positive change indicates improvement.

FIG. 36: Change from baseline pridopidine vs. placebo in Articulation rate (syllables/second) to 24 weeks, definite+probable ALS, MMRM Model. Pridopidine demonstrates a significant improvement in definite+probable subjects (change vs placebo 0.32, p=0.0002). The effect is larger in subjects who are early with <18 months from symptom onset (change vs. placebo 0.44, p=0.001) and larger and more statistically significant in subjects who are faster progressors with pre-baseline slope ≥1 (change vs. placebo 0.85, p=0.00004). The largest, significant effect seen in definite ALS subjects who are early with <18 months from symptom onset and faster progressors with pre-baseline slope ≥1 (change vs placebo 1.03, p=0.0002). Positive change indicates improvement.

FIG. 37: Percent change from baseline pridopidine vs. placebo at week 24 in serum levels of Neurofilament Light (NfL) protein, MMRM model. Serum levels of NfL protein were log-transformed and percent change of Geometric LSMean ratio from baseline was calculated and compared to placebo. Graph illustrates the percent in change of Geometric LS Means ratio from baseline vs. placebo in serum NfL levels (log pg/mL) at week 24. In the full analysis set (FAS), pridopidine demonstrates a −4% reduction from baseline vs. placebo in NfL levels (p=0.59, negative change indicates improvement). This change is larger in subjects who are early with <18 months from symptom onset (−7%, p=0.65) and largest in subjects with who are faster progressors with pre-baseline slope ≥0.75 (change vs. placebo −16%, p=0.4)

FIGS. 38A-38B: Change from baseline in serum NfL levels per visit. Serum levels of NfL protein were log-transformed and percent change of Geometric LSMean ratio from baseline was calculated and compared to placebo. Graph illustrates the percent in change of Geometric LS Means ratio from baseline vs. placebo in serum NfL levels (log pg/mL) at 8, 16 and 24 weeks. FIG. 38A: Change from baseline in serum NfL levels per visit, FAS. Pridopidine demonstrates a decrease in serum NfL levels from baseline vs. placebo at 16 weeks (change from baseline −1% vs. +3% in placebo, negative change indicates improvement) and at 24 weeks (−3% vs. +1% in placebo, p=0.59). FIG. 38B: Change from baseline in serum NfL levels per visit, FAS who are faster progressors with pre-baseline slope ≥0.75. Pridopidine demonstrates less increase in serum NfL levels from baseline vs. placebo at 8 weeks (+2% vs. +5% in placebo), at 16 weeks (0% vs. +5% in placebo) and a decrease at 24 weeks (−13% vs. +4% in placebo, p=0.4).

FIG. 39: Percent change from baseline in serum levels of Serum Neurofilament Light (NfL) protein to 24 weeks, definite+probable ALS, MMRM model. Serum levels of NfL protein were log-transformed and percent change of Geometric LSMean ratio from baseline was calculated. Graph illustrates the percent in change of Geometric LS Means ratio from baseline in serum NfL levels (log pg/mL) in definite+probable ALS subjects. In definite+probable ALS, there is no change at 24 weeks in the placebo group in NfL levels, and pridopidine demonstrates a −6% change from baseline (negative change indicates improvement). In definite+probable subjects who are early with <18 months from symptom onset, placebo group shows an increase in NfL levels (+4%), while pridopidine decreases NfL levels (−5%). This change is larger in definite +probable subjects who are faster progressors with pre-baseline slope ≥1 (−2% in placebo vs. −28% in pridopidine groups). The largest effect is seen in definite and probable subjects who are early with <18 months from symptom onset and faster progressors pre-baseline slope ≥1 (+8% in placebo vs. −35% in pridopidine group).

FIG. 40: Percent change from baseline pridopidine vs. placebo in serum levels of Neurofilament Light (NfL) protein to 24 weeks, definite+probable ALS, MMRM model. Serum levels of NfL protein were log-transformed and percent change of Geometric LSMean ratio from baseline vs. placebo was calculated. In definite+probable ALS subjects, pridopidine decreased NfL levels vs placebo by −6.1% (negative change indicates improvement, p=0.62). This effect is larger in definite+probable subjects who are early <18 months from symptom onset (change vs placebo −9%, p=0.75). A larger effect is observed in definite+probable subjects who are faster progressors with pre-baseline slope ≥1 (change vs. placebo −27%, p=0.51). The largest effect is observed in definite+probable subjects with who are early <18 months from symptom onset and faster with pre-baseline slope ≥1 (change vs. placebo −40%, p=0.49).

FIGS. 41A-41B: association between change in NfL and change in ALSFRS-R Total Score at 24 weeks. FIG. 41A: Pridopidine effect on association between Δ log NfL and ΔALSFRS-R Total Score, FAS. Graph demonstrates slope of ΔNfL levels and ΔALSFRS-R Total score in the placebo and pridopidine groups in FAS, who are early with <18 months from symptom onset and faster with a pre-baseline slope ≥1. In the placebo group, a significant negative association between worsening in ALSFRS-R and increased levels of NfL (slope −3.06, p=0.043) is observed. In contrast, pridopidine flattens the slope, indicating less decline in ALSFRS-R and reduction in NfL levels, at 24 weeks (slope 0.17, p=0.92). FIG. 41B: association between change in NL and change in ALSFRS-R Total Score at 24 weeks, definite+probable ALS. Graph demonstrates the slope of ΔNfL levels and ΔALSFRS-R Total score in the placebo and pridopidine groups in definite+probable, <18 months from symptom onset and pre-baseline slope ≥1. In the placebo group, a significant negative association between worsening in ALSFRS-R and increased levels of NfL at 24 weeks (slope −3.25, p=0.046) observed. In contrast, pridopidine flattens the slope, indicating less decline in ALSFRS-R Total and decrease in NfL levels (slope 0.61, p=0.74).

FIG. 42: Presents Product-Limit Survival Estimates with number of subjects at risk and 95% Hall-Wellner bands. Pridopidine shows a clinical meaningful effect on survival probability compared to placebo.

FIG. 43: Significant increase in survival in patients treated with pridopidine compared to matched subjects from PRO-ACT.

FIG. 44: Significant increase in survival in Definite, Probable and Early Subgroup of ALS patients treated with pridopidine compared to matched subjects from PRO-ACT.

### DETAILED DESCRIPTION OF THE INVENTION

The invention provides for a method for treating amyotrophic lateral sclerosis (ALS) in a subject, comprising administering to the subject an amount of pridopidine or pharmaceutically acceptable salt thereof effective to treat the subject.

ALS diagnosis is done by El Escorial. Diagnosis of ALS involves an in-depth evaluation and multiple diagnostic tests. The definitive diagnosis is established by considering the progressive upper (UMN) and lower motor neuron (LMN) loss. (Brooks, B. R., 1994).

Clinically Definite ALS according to El Escorial is defined on clinical evidence alone by the presence of UMN, as well as LMN signs, in three regions.

Clinically Probable ALS according to El Escorial is defined on clinical evidence alone by UMN and LMN signs in at least two regions with some UMN signs necessarily rostral to (above) the LMN signs.

Clinically Probable with Labs according to El Escorial is defined when clinical signs of UMN and LMN dysfunction are in only one region, or when UMN signs alone are present in one region, and LMN signs defined by EMG criteria are present in at least two limbs, with proper application of neuroimaging and clinical laboratory protocols to exclude other causes.

Clinically Possible ALS according to El Escorial is defined when clinical signs of UMN and LMN dysfunction are found together in only one region or UMN signs are found alone in two or more regions; or LMN signs are found rostral to UMN signs and the diagnosis of Clinically Probable—Laboratory-supported ALS cannot be proven by evidence on clinical grounds in conjunction with electrodiagnostic, neurophysiologic, neuroimaging or clinical laboratory studies. Other diagnoses must have been excluded to accept a diagnosis of Clinically Possible ALS

In some embodiments, the ALS subject to be treated is defined as clinically definite ALS. In some embodiments the ALS subject is defined as clinically probable ALS. In some embodiments, the ALS subject is defined as clinically probable with labs. In some embodiments the ALS subject is defined as clinically possible ALS.

In some embodiments provided herein, the subject population the subject to be treated is based on a TRICALS Risk Score, assessed using the ENCALS prognostic model. In certain embodiments, subjects are selected if they meet a predefined TRICALS/ENCALS risk threshold suitable for identifying patients with a desired predicted disease trajectory.

In some embodiments provided herein, the subject to be treated is selected according to respiratory-function criteria. In certain embodiments, subjects are selected if they exhibit a Slow Vital Capacity (SVC) of ≥60% predicted, measured in accordance with the GLI-2012 reference equations.

In some embodiments, the method comprises administering the composition to subjects who meet at least one of the following criteria: (i) a predefined TRICALS/ENCALS risk threshold: or (ii) an SVC ≥60% predicted (GLI-2012). In certain embodiments, subjects may be selected who meet both criteria.

In some embodiments, the method comprises administering the composition to subjects who meet at least one of the following criteria: TRICALS Risk Score between >6 and <−2, a disease duration ≤18 months from symptom onset, and a Slow Vital Capacity (SVC)≥60% of predicted according to GLI-2012 standards.

The invention further provides a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof.

In some embodiments the ALS is limb-onset or a bulbar onset.

Bulbar signs include major impacts on speech, swallowing, and quality of life. Bulbar signs can either be the presenting symptoms (in the case of bulbar onset) or appear in later stages of the disease. Bulbar-onset ALS subjects often experience a more severe form of the disease, with rapid progression and shorter survival.

### Respiratory Function

Respiratory impairment is a key feature of ALS, which results from weakening of the respiratory musculature, leading to decreased lung capacity, reduced airflow, and increased difficulty breathing. This can result in respiratory failure and potentially life-threatening complications. Many patients will require assisted ventilation at advanced stages of the disease. Respiratory failure is the leading cause of death in ALS. Thus, respiratory function is a critical predictor of survival in ALS. Early identification and monitoring of respiratory symptoms can provide information on the expected rate of progression and inform treatment plans.

In ALS, respiratory and speech are two closely interrelated functions that are commonly affected. Respiratory dysfunction in ALS can lead to speech difficulty as well as increased breathing difficulties. As the disease progresses, both speech and respiratory function may decline, leading to worsening disability and impaired quality of life.

Respiratory function in ALS patients is commonly assessed by forced vital capacity (FVC) and/or slow vital capacity (SVC). These measures provide information about the subject's lung function and ability to inhale and exhale air.

FVC measures how quickly air can be expelled from the lungs. Can predict hypoventilation, functional decline, and survival.

SVC measures the amount of air expelled from the lungs during a slow, gentle breath. It can predict survival and disease progression.

SVC and FVC are strongly correlated and decline similarly in ALS (about 2% per month). (Pinto S, de Carvalho M. SVC Is a Marker of Respiratory Decline Function, Similar to FVC, in Patients With ALS. Front Neurol. 2019 Feb. 28; 10:109).

### Quantitative Voice Analysis

Quantitative voice analysis involves using advanced speech analysis technology to objectively measure changes in speech and voice quality in individuals with ALS.

ALS causes speech difficulties in the majority (80-95%) of patients, leading to the need for augmentative and alternative communication methods. The loss of effective communication can result in psychological and social problems and decreased quality of life. Different parameters of speech can be measured, including speaking rate maximum phonation time, pause rate, breathy vocal quality, pitch instability, regulation of voicing, articulatory precision, articulation rate and monotonicity.

### Speaking Rate (Syllable/Sec):

Speaking rate refers to the number of syllables produced in each time, and a decrease in this rate can lead to difficulties in communicating effectively. Decline in Speaking rate can have a significant impact on a person's quality of life and ability to communicate. Speaking rate is an important factor that affects the abilities of individuals with ALS.

### Phonation Time:

Phonation time refers to the amount of time a person can produce sound during speech, and a decrease in this time can lead to difficulties in speech production.

### Articulation Rate (Syllables/Sec):

Articulation rate refers to the speed at which speech sounds are produced. It is measured in terms of the number of syllables or speech sounds produced in a given period of time. A typical adult has an articulation rate of about 150-160 syllables per minute. A slower articulation rate can result in speech that is difficult to understand. In the context of ALS, a reduction in articulation rate can result from the degeneration of the motor neurons that control the muscles responsible for speech production, leading to a slowing of speech sounds. This reduction in articulation rate can have a significant impact on the intelligibility and clarity of speech, making it challenging for listeners to understand what is being said. The measurement of articulation rate can be used as an indicator of the progression of ALS and can be useful in monitoring the effectiveness of speech therapy interventions aimed at improving speech intelligibility.

### Articulation Precision:

Articulation precision refers to the ability to produce speech sounds correctly and distinctly. In the context of ALS, articulation precision is used as a measure of speech function to assess the impact of the disease on speech abilities. The decline of articulation precision can indicate bulbar involvement and disease progression in ALS subjects. Measurements of articulation precision can be useful in monitoring changes in speech function and guiding treatment decisions for people with ALS.

### Measuring Clinical Progression in ALS

### ALS Functional Rating Scale—Revised (ALSFRS-R)

The ALSFRS-R (Amyotrophic Lateral Sclerosis Functional Rating Scale-Revised) is the gold standard clinical scale used to diagnose and track the progression of ALS. The ALSFRS-R comprises of 4 subdomains, each having 3 questions with scores of 0-4 (total of 12 questions per subdomain), with a maximal score of 48, corresponding to normal functionality in the three evaluated domains:
1. Bulbar function (speech, salivation and swallowing)
2. Fine Motor (handwriting, cutting food and dressing & hygiene)
3. Gross motor (turning in bed, walking and climbing stairs)
4. Respiratory function (dyspnea, orthopnea and respiratory insufficiency)

Higher scores indicate better function. Total ALSFRS-R score is obtained by summing scores from all questions, providing a comprehensive assessment of functional abilities in ALS.

### Center for Neurologic Study—Bulbar Function Scale (CNS-BFS)

Central Nervous System—Bulbar Function Scale (CNS-BFS) is a 21-item instrument completed by participants for assessing bulbar function in three domains: speech, swallowing, and sialorrhea. For each domain, participants are asked to rate the degree to which each of seven statements describing an aspect of bulbar dysfunction apply to the participant's personal experience over the past week on a scale from 1 (“Does not apply”) to 5 (“Applies most of the time”). Subjects unable to speak are assigned a value of 6 for each item comprising the speech domain. The total score is the sum of all items (range 21 to 112). Higher scores indicate worse bulbar dysfunction.

The CNS-BFS can help clinicians monitor the progression of bulbar symptoms and track changes in function over time, which can inform treatment decisions and provide important insights into the overall prognosis of the subject.

### Slow Vital Capacity (SVC) and Forced Vital Capacity (FVC)

The SVC is measured using a portable spirometer. Slow vital capacity (SVC) is the maximum volume of air that can be slowly exhaled after slow, maximal inhalation.

FVC is the maximum volume expired and converted to percent of predicted normal using normal values for FVC. Higher values indicate greater respiratory function. FVC normal values are calculated based on sex, age at time of assessment, height at time of screening, and race using equations published by the Global Lung Function Initiative (GLI; Quanjer et al. 2012).

In some embodiments, the ALS is sporadic ALS.

In some embodiments, the ALS is familial ALS (FALS). In some embodiments the ALS is juvenile ALS (JALS).

In some embodiments the ALS is not FALS. In some embodiments the ALS is not juvenile ALS (JALS).

In some embodiments, the type of ALS is classic, bulbar, flail arm, flail leg, pyramidal and respiratory ALS, progressive muscular atrophy, primary lateral sclerosis or progressive bulbar palsy.

In some embodiments, the subject carries a mutant version of a gene that causes or contributes to ALS pathogenesis. In some embodiments the mutant version of the gene is selected from the group of genes consisting of the superoxide dismutase 1 (SOD1), TAR DNA-binding protein (TARDBP) encoding TDP-43, fused in sarcoma (FUS), p62 (SQSTM1), ubiliquin-2 (UBQLN2), TANK-binding kinase 1 (TBK1), profilin 1 (PFN1), VCP or p97 (VCP), angiogenin (ANG), optineurin (OPTN), C9orf72, Sigma-1 Receptor (S1R), Tubulin alpha-4A (TUBA4A), Dynactin (DCTN1), hnRNPA1 (HNRNPA1), Matrin 3 (MATR3), Coiled-coil-helix-coiled-coil-helix domain containing 10 (CHCHD10) genes and any combination thereof.

In some embodiments, maintaining, improving, or lessening the decline of ALS patient's functionality comprises maintaining, improving, or lessening the decline of speech, salivation, swallowing, handwriting, cutting food and handling utensils, dressing and hygiene, turning in bed and adjusting bed clothes, walking, climbing stairs, dyspnea, orthopnea, respiratory insufficiency, or any combination thereof in ALS patients.

In some embodiments, the change in respiratory function is assessed by slow vital capacity (SVC). In some embodiments, the change in respiratory function is expressed as a change/month (slope). In some embodiments, the improvement is observed as a change/month of 0.2-0.5%. In other embodiments, the improvement is observed as a change/month of 0.5-2.5%. In other embodiments, the improvement is observed as a change/month of 2-5%. In some embodiments, the change/months in SVC % is over 5%.

In some embodiments, the change in respiratory function is assessed by full vital capacity (FVC). In some embodiments, the change in respiratory function is expressed as a change/month (slope). In some embodiments, the improvement is observed as a change/month of 0.2-0.5%. In other embodiments, the improvement is observed as a change/month of 0.5-2.5%. In other embodiments, the improvement is observed as a change/month of 2-5%. In some embodiments, the change/months in FVC % is over 5%.

In some embodiments, the maintaining, improving, or lessening the decline in muscle strength is measured isometrically using hand-held dynamometry (HHD), bilateral Hand Grip or combination thereof.

In an embodiment of the invention, the subject has bulbar dysfunction.

In some embodiments, the maintaining, improving, or lessening the decline in bulbar function is measured by the ALSFRS-R bulbar subdomain (Q1-Q3) score. In some embodiments, the change in bulbar function is expressed as a change/month (slope).

In some embodiments, the maintaining, improving, or lessening the decline in bulbar function is measured by the CNS-BFS. In some embodiments, the change in bulbar function is expressed as a change/month (slope).

In some embodiments, the subject has rapid pre-baseline progression wherein the pre-baseline progression is expressed by the ALSFRS-R slope. In other embodiments, the pre-baseline slope in ALSFRS-R (delta-FRS) is defined as 48 minus the baseline ALSFRS-R total score then divided by the number of months from onset of symptomatic weakness to the Baseline Visit.

In some embodiments the ALS subject is defined as a faster progressor. In some embodiments the ALS subject is defined as a faster progressor based on ALSFRS-R pre-baseline slope. In other embodiments the ALS subject has a pre-baseline slope ≥0.75. In other embodiments, the ALS subject has a pre-baseline slope of ≥0.9. In other embodiments, the ALS subject has a pre-baseline slope of ≥0.95. In other embodiments, the ALS subject has a pre-baseline slope of ≥1.

In some embodiments the ALS subject is defined as an early ALS subject. In other embodiments early ALS is defined by time from symptom onset. In some embodiments, the early ALS subject is <12 months from symptom onset. In some embodiments, the early ALS subject is <18 months from symptom onset. In some embodiments the early ALS subject is <20 months from symptom onset. In some embodiments the ALS subject is <24 months from symptom onset.

In some embodiments, the subject is an early ALS subject and faster progressor. In other embodiments, the ALS subject is <12 months from symptom onset, with a pre-baseline slope ≥0.75. In other embodiments, the ALS subject is <12 months from symptom onset, with a pre-baseline slope ≥0.9. In other embodiments, the ALS subject is <12 months from symptom onset, with a pre-baseline slope ≥0.95. In other embodiments, the ALS subject is <12 months from symptom onset, with a pre-baseline slope ≥1. In other embodiments, the ALS subject is <18 months from symptom onset, with a pre-baseline slope ≥0.75. In other embodiments, the ALS subject is <18 months from symptom onset, with a pre-baseline slope ≥0.9. In other embodiments, the ALS subject is <18 months from symptom onset, with a pre-baseline slope ≥0.95. In other embodiments, the ALS subject is <18 months from symptom onset, with a pre-baseline slope ≥1. In other embodiments, the ALS subjects is <20 months from symptom onset, with a pre-baseline slope ≥0.75. In other embodiments, the ALS subject is <20 months from symptom onset, with a pre-baseline slope ≥0.9. In other embodiments, the ALS subject is <20 months from symptom onset, with a pre-baseline slope ≥0.95. In other embodiments, the ALS subject is <24 months from symptom onset, with a pre-baseline slope ≥1. In other embodiments, the ALS subject is <14 months from symptom onset, with a pre-baseline slope ≥0.75. In other embodiments, the ALS subject is <24 months from symptom onset, with a pre-baseline slope ≥0.9. In other embodiments, the ALS subject is <24 months from symptom onset, with a pre-baseline slope ≥0.95. In other embodiments, the ALS subject is <24 months from symptom onset, with a pre-baseline slope ≥1.

In some embodiments, the amount of pridopidine is effective to change time to first evidence of bulbar dysfunction.

In some embodiments, the maintaining, improving, or lessening the decline in speech is measured by the CNS-BFS Speech domain.

In an embodiment of the invention, the maintaining, improving, or lessening the decline in speech is measured by the ALSFRS-R speech subdomain score (Q1).

Several studies have found that speech features, such as jitter, shimmer, articulatory rate, speaking rate, and pause rate, are affected in ALS. In some embodiments of the invention, use of pridopidine maintains, improves or lessens the decline in speech characteristics as measured by automated algorithmic assessment of speech collected digitally as described in Stegmann, G. et al., 2020 which is incorporated herein by reference.

In some embodiments, the maintaining, improving, or lessening the decline in speech is measured by automated algorithmic assessment of speech collected digitally.

In some embodiments, the effect on speech is measured by articulation rate (syllables/second). In other embodiments, the effect on speech is measured by speaking rate (syllables/sec). In other embodiments, the effect on speech is measured in phonation time (sec). In other embodiments, the effect on speech is measured by max phonation time (sec). In other embodiments, the effect on speech is measured by pause rate. In other embodiments, the effect on speech is measured by breathy vocal quality. In other embodiments, the effect on speech is measured by pitch instability. In other embodiments, the effect on speech is measured by regulation of voicing. In other embodiments, the effect on speech is measured by monotonicity. In other embodiments, the effect on speech is measured by articulatory precision (ratio). In other embodiments, the effect on speech is measured by articulation rate (syllables/second).

A technology for assessing and evaluating the following parameters:
1. Articulation rate: The speed at which a person speaks, measured in syllables or words per minute.
2. Jitter: A measure of the variability in the duration of successive speech sound units.
3. Shimmer: A measure of the variability in the amplitude (volume) of successive speech sound units.
4. Voice onset time: The time from the initiation of a speech sound to the onset of vocal fold vibration.
5. Phonation time ratio: The ratio of the time of voice to the time of silence in a speech sample.

In an embodiment of the invention, the maintaining, improving, or lessening the decline of ALS as measured by the ALS Functional Rating Scale-Revised (ALSFRS-R). In some embodiments, the change in ALSFRS-R is expressed as a change/month (slope).

In an embodiment of the invention, the amount of pridopidine is effective to improve, maintain, or lessen the decline of a symptom of the ALS in the subject. In some embodiments, the progression of a symptom is expressed as a change/month (slope).

In some embodiments, the amount of a composition comprising pridopidine is effective to maintain, reduce or lessen the increase in neurofilament light (NfL) protein levels. In some embodiments, the amount of a composition comprising pridopidine is effective to maintain NfL levels. In some embodiments, a composition comprising pridopidine is effective to reduce neurofilament light (NfL) protein levels by more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, >80%. In some embodiments a composition comprising pridopidine is effective lessen the increase in neurofilament light (NfL) protein levels by more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80% compared to untreated ALS subjects.

In some embodiments, the symptom of ALS is a clinical symptom of ALS.

In some embodiments, the symptom of ALS is muscle weakness and hypotrophy, fasciculations and cramps, spastic hypertonus, hyperreflexia, dysarthria, dysphagia and respiratory weakness, behavioral disturbances, dysexecutive impairment, or frontotemporal dementia.

In some of embodiments of the invention, the symptom of ALS is a neuropathological symptom.

In some embodiments, the symptom is bulbar palsy or pseudobulbar affect (PBA).

In some embodiments, the symptom of ALS is muscle atrophy, loss of motor neurons, loss of anterior horn cells, sclerosis of the spinal cord lateral columns, or gliosis.

In some embodiments, the symptom of ALS is a rate of decline (a) in pulmonary function, (b) in functional disability, or (c) in the ability score for the lower extremities. In an embodiment of the invention, the amount of pridopidine is effective to cause survival of the subject or cause neuroprotection in the subject.

In some embodiments provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof, wherein the amount of pridopidine or pharmaceutically acceptable salt thereof is effective in maintaining, reducing or lessening the increase in GFAP levels in a human subject afflicted with ALS.

In some embodiments provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof, wherein the amount of pridopidine or pharmaceutically acceptable salt thereof is effective in maintaining, reducing or lessening the increase in YKL-40 levels in a human subject afflicted with ALS.

In some embodiments provided herein, the method further comprises wherein the method further comprises assessing a biomarker associated with ALS progression or treatment response: wherein the biomarker comprises cardiac Troponin T (cTnT), and wherein the subject has elevated cTnT levels at baseline, demonstrates a longitudinal change in cTnT levels during treatment, or is stratified according to cTnT levels for assessing disease severity or progression rate. In other embodiments, the subject has elevated cTnT levels at baseline. In other embodiments, the subject demonstrates a longitudinal change in cTnT levels during treatment. In other embodiments, cTnT levels are used to stratify subjects according to disease severity or progression rate.

In some embodiments provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof, wherein the maintaining, improving, or lessening the decline is measured by the ALS Functional Rating Scale-Revised (ALSFRS-R).

In some embodiments provided herein, the method further comprises assessing treatment efficacy using patient-reported outcomes (PROs). In certain embodiments, the PRO evaluation includes the Patient Reported Outcome of Function (PROOF) instrument to capture patient-perceived functional benefit. In another embodiment the treatment efficacy is measured by ALSFRS-R Patient-Ranked Order of Function (PROOF), the ALS Assessment Questionnaire-40 (ALSAQ-40), the Communicative Participation Item Bank (CPIB), the EQ-5D-5L including EQ-VAS or combination thereof.

In some embodiments provided herein, the method comprises evaluating communication-related quality of life using the Communicative Participation Item Bank (CPIB). In certain embodiments, CPIB scores are incorporated to quantify the subject's perceived ability to participate in communicative activities.

In some embodiments, the method includes assessing general health-related quality of life using the EQ-5D-5L instrument. In certain embodiments, the EQ-5D-5L index value and visual analogue scale (VAS) are used to evaluate changes in overall well-being.

In some embodiments provided herein, the method further comprises measuring ALS-specific quality of life using the ALSAQ-40 questionnaire. In certain embodiments, ALSAQ-40 domain scores are used to capture patient-perceived changes in mobility, activities of daily living, eating and drinking, communication, and emotional functioning.

In some embodiments provided herein, the method further comprises analyzing an estimate that is adjusted for mortality, in accordance with the principles set forth in ICH E9(R1). Such an approach provides a rigorous, transparent, and clinically meaningful assessment of treatment effect in subjects with ALS.

In some embodiments provided herein, the method further comprises evaluating survival-related outcomes using a predefined operational definition of survival status.

In some embodiments provided herein, survival outcomes analyzed by treating tracheostomy or PAV>22 hours/day for >7 days as death-equivalent events.

In certain embodiments, the term “death equivalent” is defined as the occurrence of tracheostomy in the subject.

In some embodiments, a “death equivalent” is defined as the initiation of permanent assisted ventilation (PAV) for more than 22 hours per day for a duration exceeding 7 consecutive days.

In some embodiments, provided herein a method for decreasing the risk of death or prolonging the survival of a subject with ALS wherein the method comprises administering to the subject a composition comprising a therapeutically acceptable amount of pridopidine or pharmaceutically acceptable salt thereof.

In some embodiments, provided herein a method for decreasing the risk of death of a subject with ALS wherein the method comprises administering to the subject a composition comprising a therapeutically acceptable amount of pridopidine or pharmaceutically acceptable salt thereof for at least 4 months. In another embodiment for at least 6 months. In another embodiment for at least 8 months. In another embodiment for at least 10 months. In another embodiment for between 4 to 24 months. In another embodiment, chronically.

In some embodiments, provided herein a method for decreasing the risk of death of a subject with ALS wherein the method comprises administering to the subject a composition comprising a therapeutically acceptable amount of pridopidine or pharmaceutically acceptable salt thereof, wherein the decrease in the risk of death is between 20%-90% compared to a subject not treated with pridopidine. In another embodiment, the decrease in the risk of death is between 30%-60% compared to a subject not treated with pridopidine. In another embodiment, the decrease in the risk of death is between 50%-70% compared to a subject not treated with pridopidine. In another embodiment, the decrease in the risk of death is about 20%, 30%, 40%, 50%, 60%, 70%, 80%, 90% or any ranges thereof compared to a subject not treated with pridopidine.

In some embodiments, provided herein a method prolonging the survival of a subject with ALS wherein the method comprises administering to the subject a composition comprising a therapeutically acceptable amount of pridopidine or pharmaceutically acceptable salt thereof for at least 4 months. In another embodiment for at least 6 months. In another embodiment for at least 8 months. In another embodiment for at least 10 months. In another embodiment for between 4 to 24 months. In another embodiment, chronically.

In another embodiment, the survival is further evaluated through week 96, with death equivalents defined as tracheostomy or permanent assisted ventilation (>22 hours/day for >7 consecutive days).

In some embodiments, provided herein a method prolonging the survival of a subject with ALS wherein the method comprises administering to the subject a composition comprising a therapeutically acceptable amount of pridopidine or pharmaceutically acceptable salt thereof, wherein the method prolongs the survival of a subject with ALS by at least between 2-24 months compared to a subject not treated with pridopidine. In another embodiment, the method prolongs the survival of a subject with ALS by at least 2 months compared to a subject not treated with pridopidine. In another embodiment, the method prolongs the survival of a subject with ALS by at least 4 months compared to a subject not treated with pridopidine. In another embodiment, the method prolongs the survival of a subject with ALS by at least 6 months compared to a subject not treated with pridopidine. In another embodiment, the method prolongs the survival of a subject with ALS by at least 8 months compared to a subject not treated with pridopidine. In another embodiment, the method prolongs the survival of a subject with ALS by at least 10 months compared to a subject not treated with pridopidine. In another embodiment, the method prolongs the survival of a subject with ALS by at least 12 months compared to a subject not treated with pridopidine. In another embodiment, the method prolongs the survival of a subject with ALS by at least 14 months compared to a subject not treated with pridopidine. In another embodiment, the method prolongs the survival of a subject with ALS by at least 16 months compared to a subject not treated with pridopidine. In another embodiment, the method prolongs the survival of a subject with ALS by at least 18 months compared to a subject not treated with pridopidine. In another embodiment, the method prolongs the survival of a subject with ALS by at least 20 months compared to a subject not treated with pridopidine. In another embodiment, the method prolongs the survival of a subject with ALS by at least 22 months compared to a subject not treated with pridopidine. In another embodiment, the method prolongs the survival of a subject with ALS by at least 24 months compared to a subject not treated with pridopidine.

In some embodiments of the invention, treatment of the subject with pridopidine results in a lessened decline, maintenance or an improvement in the subject, in one or more of the following domains, 1) speech, 2) salivation, 3) swallowing, 4) handwriting, 5) cutting food and handling utensils (with or without gastrostomy), 6) dressing and hygiene, 7) turning in bed and adjusting bed clothes, 8) walking, 9) climbing stairs, 10) breathing, 11) dyspnea, 12) orthopnea, and 13) respiratory insufficiency.

In some embodiments, patients are monitored for changes in the above domains using a rating scale, for example the Amyotrophic Lateral Sclerosis Functional Rating Scale (ALSFRS) or revised ALSFRS (ALSFRS-R) and a functional change in a patient is monitored over time.

In some embodiments, pseudobulbar affect (PBA) (as measured by CNS-LS) is monitored in the patients. In some embodiments, the severity and/or frequency of emotional outbursts in subjects experiencing PBA is reduced with pridopidine treatment.

In some embodiments, the use of pridopidine improves, maintains or lessens the decline of a disease severity as measured by the ALS Functional Rating Scale-Revised (ALSFRS-R) in ALS patients and/or ALSAQ-5.

In some embodiments, use of pridopidine or pharmaceutically acceptable salt thereof improves, maintains or lessen the decline in respiratory function as assessed by slow vital capacity (SVC) in ALS patients. In some embodiments, the change in SVC is expressed as a percent change/month (slope). In some embodiments of the invention, use of pridopidine improves, maintains or lessens the decline in respiratory function as assessed by full vital capacity (FVC) in ALS patients. In some embodiments of the invention, use of pridopidine improves, maintains, or lessens the decline in respiratory function as assessed by ALSFRS-R Respiratory subdomain.

In some embodiments of the invention, use of pridopidine or pharmaceutically acceptable salt thereof for lessening the decline in muscle strength as measured by handheld dynamometry (HHD) in ALS patients.

In some embodiments of the invention, use of pridopidine or pharmaceutically acceptable salt thereof for maintaining, reducing, or lessening the increase in phosphorylated neurofilament heavy chain (pNfH) and neurofilament light chain (NfL) in plasma, serum and CSF in ALS patients.

In some embodiments of the invention, use of pridopidine or pharmaceutically acceptable salt thereof results in maintenance, reduction or less increase in urinary neurotrophin receptor p75 extracellular domain (p75^{ECD}) in ALS patients.

In some embodiments of the invention, use of a composition comprising pridopidine or pharmaceutically acceptable salt thereof in combination with sodium phenylbutyrate (PB), tauroursodeoxycholic acid, combination of sodium phenylbutyrate (PB)/tauroursodeoxycholic acid, DNL343, Trehalose (SLS-005), CNM-Au8 nanocrystalline gold, ABBV-CLS-7262 or combination thereof, results in maintenance, reduction or less increase in phosphorylated neurofilament heavy chain (pNfH) and neurofilament light chain (NfL) ALS patients for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof.

In some embodiments of the invention, use of a composition comprising pridopidine or pharmaceutically acceptable salt thereof in combination with sodium phenylbutyrate (PB), tauroursodeoxycholic acid, combination of sodium phenylbutyrate (PB)/tauroursodeoxycholic acid, DNL343, Trehalose (SLS-005), CNM-Au8 nanocrystalline gold, ABBV-CLS-7262 or combination thereof, for maintenance, reduction or lessen the increase in urinary neurotrophin receptor p75 extracellular domain (p75^{ECD}) in ALS patients.

In some embodiments of the invention, use of a composition comprising pridopidine or pharmaceutically acceptable salt thereof in combination with sodium phenylbutyrate (PB), tauroursodeoxycholic acid, combination of sodium phenylbutyrate (PB)/tauroursodeoxycholic acid, DNL343, Trehalose (SLS-005), CNM-Au8 nanocrystalline gold, ABBV-CLS-7262 or combination thereof, for maintenance, reduction or lessen the increase in troponin I and/or troponin T in plasma and CSF in ALS patients.

In some embodiments of the invention, use of pridopidine or pharmaceutically acceptable salt thereof maintains, improves, or lessens the decline in speech characteristics as measured by the slope of change in the CNS-BFS speech subdomain in ALS patients.

Several studies have found that speech features, such as jitter, shimmer, articulatory rate, speaking rate, and pause rate, are affected in ALS. In some embodiments of the invention, use of pridopidine maintains, improves, or lessens the decline in speech characteristics as measured by automated algorithmic assessment of speech collected digitally. Automated algorithmic assessment of speech is described in Stegmann, G. et al., 2020 which is incorporated herein by reference.

In some embodiments of the invention, use of pridopidine or pharmaceutically acceptable salt thereof in ALS patients maintains, improves, or lessens the decline in voice characteristics as determined by Aural Analytics set of analyses in ALS patients.

In some embodiments of the invention, use of pridopidine or pharmaceutically acceptable salt thereof maintains, improves, or lessens the decline in cognitive function as measured by the Edinburgh Cognitive and Behavioral ALS Screen (ECAS) in ALS patients.

In some embodiments of the invention, use of pridopidine or pharmaceutically acceptable salt thereof maintains, improves, or lessens the decline in home-based clinical assessments (weekly ALSFRS-R, SVC, home spirometry FVC pinch strength) in ALS patients.

In some embodiments, use of pridopidine or pharmaceutically acceptable salt thereof maintains, improves, or lessens the decline in bulbar function as measured by the CNS-BFS (Center for Neurologic Study Bulbar Function Scale) and the bulbar sub-domain (Q1-Q3) score of the ALSFRS-R total score in ALS patients. In some embodiment, use of pridopidine or pharmaceutically acceptable salt thereof maintains, improves, or lessens the decline in swallowing as measured by the bulbar sub-domain score of the ALSFRS-R ALS patients. In some embodiments, use of pridopidine or pharmaceutically acceptable salt thereof maintains, improves, or lessens the decline in salivation as measured by the bulbar sub-domain score of the ALSFRS-R ALS patients. In some embodiments, use of pridopidine or pharmaceutically acceptable salt thereof maintains, improves, or lessens the decline in speech as measured by the bulbar sub-domain score of the ALSFRS-R ALS patients.

In some embodiments, use of pridopidine or pharmaceutically acceptable salt thereof maintains, improves, or lessens the decline in muscle strength, as measured isometrically using hand-held dynamometry (HHD) and grip strength in ALS patients.

In some embodiment, use of pridopidine or pharmaceutically acceptable salt thereof maintains, improves, or lessens the decline in bulbar function as measured by the slope of change in the CNS-BFS total score in ALS patients.

In some embodiment, use of pridopidine or pharmaceutically acceptable salt thereof maintains, improves or lessens the decline in bulbar function as measured by the slope of change in the CNS-BFS total score in ALS patients whose calculated ALSFRS-R slope at baseline (48-ALSFRS-R total score at baseline/time since onset) is equal to or greater than 0.75 pt/month. In some embodiments, the ALS patient has definite or probable ALS as defined by the El Escorial Criteria. In some embodiments, the ALS patient is an early patient <18 months from symptom onset. In some embodiments, the ALS patient is a faster progressor, with a pre-baseline ALSFRS-R slope of ≥1. In some embodiments, the definite or probable ALS patient is an early ALS patient <18 months from symptom onset. In some embodiments, the definite or probable ALS patient is a faster progressor with a pre-baseline ALSFRS-R slope of ≥1. In some embodiments, the definite or probable ALS patient is <18 months from symptom onset and a faster progressor with a pre-baseline ALSFRS-R slope of ≥1.

In some embodiments, use of pridopidine or pharmaceutically acceptable salt thereof reduces the percentage of ALS patients who develop bulbar symptoms by 6 months among participants without bulbar symptoms at baseline (as defined as a CNS-BFS score <30 at baseline) in the active compared to placebo groups.

In an embodiment of the invention, pridopidine is administered daily.

In some embodiments of the invention, pridopidine is administered more often than once daily.

In some embodiments of the invention, pridopidine is administered twice daily. In an embodiment of the invention, pridopidine is administered thrice daily.

In some embodiments of the invention, pridopidine is administered less often than once daily, for example, on alternate days, three times per week, twice per week or once per week.

In some embodiments of the invention, pridopidine is administered daily, twice a week, three times a week or more often than once daily.

In an embodiment of the invention, pridopidine is administered orally.

In some embodiments, a unit dose of the pharmaceutical composition contains 10-250 mg pridopidine. In some embodiments the composition comprises 45 mg, 67.5 mg, 90 mg, or 112.5 mg of pridopidine.

In an embodiment, between 10-225 mg pridopidine is administered to the patient per day. In another embodiment, between 45-180 mg pridopidine is administered to the patient per day. In another embodiment, 10 mg, 22.5 mg, 45 mg, 67.5, mg, 90 mg, 100 mg, 112.5 mg, 125 mg, 135 mg, 150 mg, or 180 mg pridopidine is administered to the patient per day.

In an embodiment, the pharmaceutical composition is administered twice per day. In another embodiment, an equal amount of the pharmaceutical composition is administered at each administration.

In an embodiment, the two doses are administered at least 6 hours apart, at least 7 hours, at least 8 hours, at least 9 hours, at least 10 hours, at least 11 hours apart. In some embodiments, the pharmaceutical composition is administered for at least 12 weeks, at least 20 weeks, at least 24 weeks, at least 26 weeks, at least 52 weeks, at least 78 weeks, or at least 96 weeks.

In an embodiment of the invention, the pridopidine is pridopidine hydrochloride.

In an embodiment of the invention, the subject is a human subject.

The invention also provides pridopidine or pharmaceutically acceptable salt thereof for use in treating a human subject afflicted with ALS.

The invention also provides a pharmaceutical composition comprising an effective amount of pridopidine or pharmaceutically acceptable salt thereof for use in treating a human subject afflicted with ALS.

The invention further provides a method for the treatment of ALS comprising administering to a subject in need thereof a composition comprising an amount of pridopidine or pharmaceutically acceptable salt thereof effective to treat the ALS.

In an embodiment, the pharmaceutical composition comprises an amount of pridopidine or pharmaceutically acceptable salt thereof, an analog of pridopidine, and an amount of a second compound, for example a compound useful in treating patients with ALS.

In an embodiment, the pharmaceutical composition comprises an amount of pridopidine or pharmaceutically acceptable salt thereof, one or more analogs of pridopidine, and an amount of a second compound, for example a compound useful in treating patients with ALS.

In an embodiment, the pharmaceutical composition comprises an amount of pridopidine or pharmaceutically acceptable salt thereof and an amount of a second compound, for example a compound useful in treating patients with ALS.

In some embodiments, the second compound is riluzole, edaravone, a combination of dextromethorphan and quinidine, laquinimod, sodium phenylbutyrate (PB), tauroursodeoxycholic acid, SLS-005 (Trehalose), DNL343, CNM-Au8 nanocrystalline gold, ABBV-CLS-7262 or combination of sodium phenylbutyrate (PB) and tauroursodeoxycholic acid (i.e. AMX0035).

In an embodiment, the pharmaceutical composition for use in treating ALS in a subject, comprises pridopidine or pharmaceutically acceptable salt thereof and at least one pridopidine's analog or pharmaceutically acceptable salt thereof of compounds of Formula 1-7:

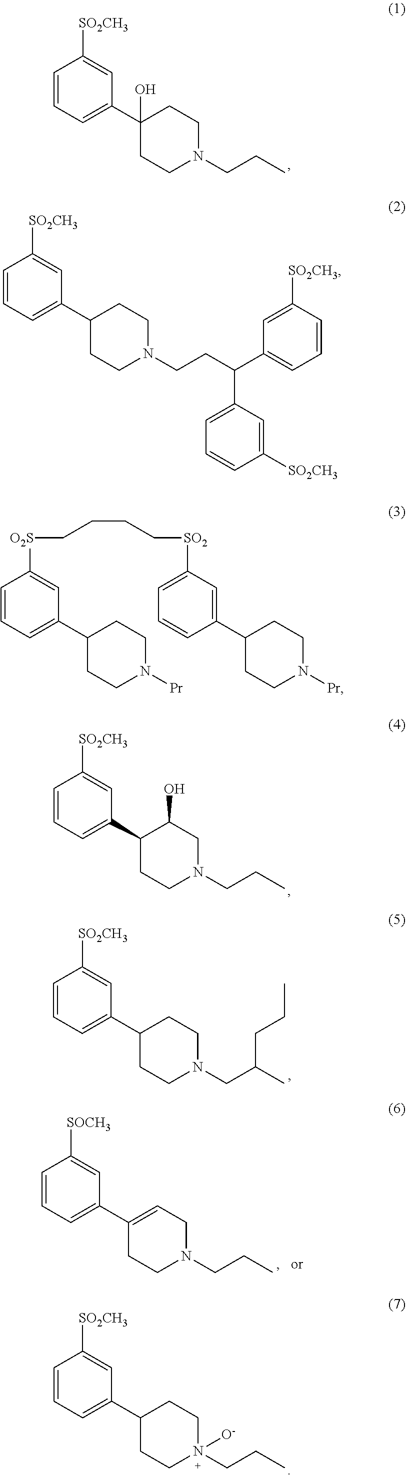

In other embodiments, the composition for use in the methods of this invention comprises pridopidine or pharmaceutically acceptable salt thereof and at least one of pridopidine's analog compounds 1-7 or pharmaceutically acceptable salt thereof. In another embodiment, the composition comprises pridopidine or pharmaceutically acceptable salt thereof and compound 1 or pharmaceutically acceptable salt thereof. In another embodiment, the composition comprises pridopidine or pharmaceutically acceptable salt thereof, compound 1 and compound 4 or pharmaceutically acceptable salt thereof. In another embodiment, the composition comprises pridopidine or pharmaceutically acceptable salt thereof and compound 2 or pharmaceutically acceptable salt thereof. In another embodiment, the composition comprises pridopidine or pharmaceutically acceptable salt thereof and compound 3 or pharmaceutically acceptable salt thereof. In another embodiment, the composition comprises pridopidine or pharmaceutically acceptable salt thereof and compound 4 or pharmaceutically acceptable salt thereof. In another embodiment, the composition comprises pridopidine or pharmaceutically acceptable salt thereof and compound 5 or pharmaceutically acceptable salt thereof. In another embodiment, the composition comprises pridopidine or pharmaceutically acceptable salt thereof and compound 6 or pharmaceutically acceptable salt thereof. In another embodiment, the composition comprises pridopidine or pharmaceutically acceptable salt thereof and compound 7 or pharmaceutically acceptable salt thereof. In another embodiment, the composition comprises pridopidine or pharmaceutically acceptable salt thereof and compound 1, 2, 3, 4, 5, 6, 7 or a pharmaceutically acceptable salt thereof or any combination thereof.

In another embodiment, the pharmaceutical composition comprising pridopidine or pharmaceutically acceptable salt and at least one of pridopidine's analog compounds 1-7 or pharmaceutically acceptable salt thereof for use in the methods of this invention, comprises between 0%-10% w/w of each of the pridopidine's analog compound relative to pridopidine. In another embodiment, the pharmaceutical composition comprises between 0.005%-5% w/w of each of the analog compounds 1-7 or pharmaceutical acceptable salt thereof relative to pridopidine. In another embodiment, the pharmaceutical composition comprises between 0.001%-1% w/w of each of the analog compounds 1-7 or pharmaceutical acceptable salt thereof relative to pridopidine. In another embodiment, the pharmaceutical composition comprises between 0.05%-3% w/w of each of the analog compounds 1-7 or pharmaceutical acceptable salt thereof relative to pridopidine. In another embodiment, the pharmaceutical composition comprises between 0.005%-0.1% w/w, 0.05%-0.1% w/w, 0.05%-0.15% w/w, 0.05%-0.2% w/w, 0.05%-0.5% w/w, 0.05%-2% w/w, 0.05%-3% w/w, 0.05%-4% w/w, 0.05%-5% w/w, 0.1%-0.4% w/w, 0.15%-0.3% w/w, 0.15%-0.5% w/w of each of the analog compounds 1-7 or pharmaceutical acceptable salt thereof relative to pridopidine.

In some embodiments, provided herein a method of treating ALS in a subject in need thereof, wherein the method comprises administering to the subject a composition comprising a therapeutically acceptable amount of pridopidine or pharmaceutically acceptable salt thereof and at least one of pridopidine's analog of compounds 1-7 or pharmaceutically acceptable salt thereof. In other embodiments, the method comprises further administering a second composition comprising a second compound which is administered simultaneously or contemporaneously with the composition comprising pridopidine and pridopidine's analog.

In some embodiments, provided herein a method of treating ALS in a subject in need thereof, wherein the method comprises administering to the subject a composition comprising a therapeutically acceptable amount of pridopidine or pharmaceutically acceptable salt thereof and Compound 1 or pharmaceutically acceptable salt thereof. In other embodiments, the method comprises further administering a second composition comprising a second compound which is administered simultaneously or contemporaneously with the composition comprising pridopidine and pridopidine's analog.

In some embodiments, provided herein a method of treating ALS in a subject in need thereof, wherein the method comprises administering to the subject a composition comprising a therapeutically acceptable amount of pridopidine or pharmaceutically acceptable salt thereof and Compound 4 or pharmaceutically acceptable salt thereof. In other embodiments, the method comprises further administering a second composition comprising a second compound which is administered simultaneously or contemporaneously with the composition comprising pridopidine and pridopidine's analog.

In some embodiments, provided herein a method of treating ALS in a subject in need thereof, wherein the method comprises administering to the subject a composition comprising a therapeutically acceptable amount of pridopidine or pharmaceutically acceptable salt thereof Compound 1 and Compound 4 or pharmaceutically acceptable salt thereof. In other embodiments, the method comprises further administering a second composition comprising a second compound which is administered simultaneously or contemporaneously with the composition comprising pridopidine and pridopidine's analog.

In some embodiments, provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof and at least one of pridopidine's analog of compounds 1-7 or pharmaceutically acceptable salt thereof. In other embodiments, administering a composition comprising a therapeutically acceptable amount of pridopidine or pharmaceutically acceptable salt thereof and Compound 1 or pharmaceutically acceptable salt thereof. In other embodiments, administering a composition comprising a therapeutically acceptable amount of pridopidine or pharmaceutically acceptable salt thereof and Compound 4 or pharmaceutically acceptable salt thereof. In other embodiments, administering a composition comprising a therapeutically acceptable amount of pridopidine or pharmaceutically acceptable salt thereof and Compound 1 and Compound 4 or pharmaceutically acceptable salt thereof. In other embodiments, the method comprises further administering a second composition comprising a second compound which is administered simultaneously or contemporaneously with the composition comprising pridopidine and pridopidine's analog.

In some embodiments, provided herein a method of treating ALS in a subject in need thereof, wherein the method comprises administering to the subject a composition comprising a therapeutically acceptable amount of at least one of pridopidine's analog of compounds 1-7 or pharmaceutically acceptable salt thereof. In other embodiments, the method comprises administering a composition comprising Compound 1 or pharmaceutically acceptable salt thereof. In other embodiments, the method comprises administering a composition comprising Compound 2 or pharmaceutically acceptable salt thereof. In other embodiments, the method comprises administering a composition comprising Compound 3 or pharmaceutically acceptable salt thereof. In other embodiments, the method comprises administering a composition comprising Compound 4 or pharmaceutically acceptable salt thereof. In other embodiments, the method comprises administering a composition comprising Compound 5 or pharmaceutically acceptable salt thereof. In other embodiments, the method comprises administering a composition comprising Compound 6 or pharmaceutically acceptable salt thereof. In other embodiments, the method comprises administering a composition comprising Compound 7 or pharmaceutically acceptable salt thereof.

In some embodiments, provided herein a method of treating ALS in a subject in need thereof, wherein the method comprises administering to the subject a composition comprising a therapeutically acceptable amount of at least one of pridopidine's analog of compounds 1-7 or pharmaceutically acceptable salt thereof and a composition comprising a Second compound which is administered simultaneously or contemporaneously with the composition comprising at least one of Compounds 1-7.

In some embodiments, provided herein a method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of at least one of pridopidine's analog of compounds 1-7 or pharmaceutically acceptable salt thereof. In other embodiments, the method comprises administering a composition comprising Compound 1 or pharmaceutically acceptable salt thereof. In other embodiments, the method comprises administering a composition comprising Compound 2 or pharmaceutically acceptable salt thereof. In other embodiments, the method comprises administering a composition comprising Compound 3 or pharmaceutically acceptable salt thereof. In other embodiments, the method comprises administering a composition comprising Compound 4 or pharmaceutically acceptable salt thereof. In other embodiments, the method comprises administering a composition comprising Compound 5 or pharmaceutically acceptable salt thereof. In other embodiments, the method comprises administering a composition comprising Compound 6 or pharmaceutically acceptable salt thereof. In other embodiments, the method comprises administering a composition comprising Compound 7 or pharmaceutically acceptable salt thereof.

In other embodiments, the Pridopidine's analog compound is Compound 1 or pharmaceutically acceptable salt thereof. In other embodiments, the analog compound is Compound 2. In other embodiments, the analog compound is Compound 3. In other embodiments, the analog compound is Compound 4. In other embodiments, the analog compound is Compound 5. In other embodiments, the analog compound is Compound 6. In other embodiments, the analog compound is Compound 7.

In an embodiment, the pharmaceutical composition is in a unit dosage form, useful in treating subject afflicted with ALS, which comprises:
a) an amount of pridopidine or a pharmaceutically acceptable salt.
b) an amount of a Second compound,
wherein the respective amounts of said Second compound and said pridopidine in said composition are effective, upon concomitant administration to said subject of one or more of said unit dosage forms of said composition, to treat the subject.

In some embodiments, the Second compound is riluzole, edaravone, combination of dextromethorphan/quinidine, sodium phenylbutyrate (PB), tauroursodeoxycholic acid, SLS-005 (Trehalose), DNL343, CNM-Au8 nanocrystalline gold, ABBV-CLS-7262 or combination of sodium phenylbutyrate (PB)/tauroursodeoxycholic acid (i.e. AMX0035).

In an embodiment, the pharmaceutical composition comprises an amount of pridopidine for use in treating a subject afflicted with ALS as an add-on therapy to a Second compound which is riluzole. In another embodiment, the pharmaceutical composition comprises an amount of pridopidine for use in treating a subject afflicted with ALS as an add-on therapy to a Second compound which is edaravone. In another embodiment, the pharmaceutical composition comprises an amount of pridopidine for use in treating a subject afflicted with ALS as an add-on therapy to a Second compound which is dextromethorphan/quinidine. In another embodiment, the pharmaceutical composition comprises an amount of pridopidine for use in treating a subject afflicted with ALS as an add-on therapy to a Second compound sodium phenylbutyrate (PB). In another embodiment, the pharmaceutical composition comprises an amount of pridopidine for use in treating a subject afflicted with ALS as an add-on therapy to a Second compound tauroursodeoxycholic acid. In another embodiment, the pharmaceutical composition comprises an amount of pridopidine for use in treating a subject afflicted with ALS as an add-on therapy to combination of sodium phenylbutyrate (PB) and tauroursodeoxycholic acid (i.e. AMX0035). In another embodiment, the pharmaceutical composition comprises an amount of pridopidine for use in treating a subject afflicted with ALS as an add-on therapy to a Second compound SLS-005 (Trehalose). In another embodiment, the pharmaceutical composition comprises an amount of pridopidine for use in treating a subject afflicted with ALS as an add-on therapy to a Second compound DNL343. In another embodiment, the pharmaceutical composition comprises an amount of pridopidine for use in treating a subject afflicted with ALS as an add-on therapy to a Second compound CNM-Au8 nanocrystalline gold. In another embodiment, the pharmaceutical composition comprises an amount of pridopidine for use in treating a subject afflicted with ALS as an add-on therapy to a Second compound ABBV-CLS-7262.

In an embodiment, the pharmaceutical composition comprises an amount of pridopidine for use in treating a subject afflicted with ALS simultaneously or contemporaneously with a Second compound which is riluzole. In another embodiment, the pharmaceutical composition comprises an amount of pridopidine for use in treating a subject afflicted with ALS simultaneously or contemporaneously with a Second compound which is edaravone. In another embodiment, the pharmaceutical composition comprises an amount of pridopidine for use in treating a subject afflicted with ALS simultaneously or contemporaneously with a Second compound which is dextromethorphan/quinidine. In another embodiment, the pharmaceutical composition comprises an amount of pridopidine for use in treating a subject afflicted with ALS simultaneously or contemporaneously with a Second compound which is laquinimod. In another embodiment, the pharmaceutical composition comprises an amount of pridopidine for use in treating a subject afflicted with ALS simultaneously or contemporaneously with a Second compound which is sodium phenylbutyrate (PB). In another embodiment, the pharmaceutical composition comprises an amount of pridopidine for use in treating a subject afflicted with ALS simultaneously or contemporaneously with a Second compound which is tauroursodeoxycholic acid. In another embodiment, the pharmaceutical composition comprises an amount of pridopidine for use in treating a subject afflicted with ALS simultaneously or contemporaneously with a combination of sodium phenylbutyrate (PB) and tauroursodeoxycholic acid (i.e. AMX0035). In another embodiment, the pharmaceutical composition comprises an amount of pridopidine for use in treating a subject afflicted with ALS simultaneously or contemporaneously with a Second compound which is SLS-005 (Trehalose). In another embodiment, the pharmaceutical composition comprises an amount of pridopidine for use in treating a subject afflicted with ALS simultaneously or contemporaneously with a Second compound which is DNL343. In another embodiment, the pharmaceutical composition comprises an amount of pridopidine for use in treating a subject afflicted with ALS simultaneously or contemporaneously with a Second compound which is CNM-Au8 nanocrystalline gold. In another embodiment, the pharmaceutical composition comprises an amount of pridopidine for use in treating a subject afflicted with ALS simultaneously or contemporaneously with a Second compound which is ABBV-CLS-7262.

In an embodiment, the pharmaceutical composition comprises an amount of a compound which is riluzole for use in treating a subject afflicted with ALS as an add-on therapy to pridopidine. In another embodiment, the pharmaceutical composition comprises an amount of a compound which is edaravone for use in treating a subject afflicted with ALS as an add-on therapy to pridopidine. In another embodiment, the pharmaceutical composition comprises an amount of a compound which is dextromethorphan/quinidine for use in treating a subject afflicted with ALS as an add-on therapy to pridopidine. In another embodiment, the pharmaceutical composition comprises an amount of a compound which is laquinimod for use in treating a subject afflicted with ALS as an add-on therapy to pridopidine. In another embodiment, the pharmaceutical composition comprises an amount of a compound which is sodium phenylbutyrate (PB) for use in treating a subject afflicted with ALS as an add-on therapy to pridopidine. In another embodiment, the pharmaceutical composition comprises an amount of a compound which is tauroursodeoxycholic acid for use in treating a subject afflicted with ALS as an add-on therapy to pridopidine. In another embodiment, the pharmaceutical composition comprises an amount of a combination of sodium phenylbutyrate (PB) and tauroursodeoxycholic acid for use in treating a subject afflicted with ALS as an add-on therapy to pridopidine. In another embodiment, the pharmaceutical composition comprises an amount of a combination of SLS-005 (Trehalose) for use in treating a subject afflicted with ALS as an add-on therapy to pridopidine. In another embodiment, the pharmaceutical composition comprises an amount of a combination of DNL343, for use in treating a subject afflicted with ALS as an add-on therapy to pridopidine. In another embodiment, the pharmaceutical composition comprises an amount of a combination of CNM-Au8 nanocrystalline gold, for use in treating a subject afflicted with ALS as an add-on therapy to pridopidine. In another embodiment, the pharmaceutical composition comprises an amount of a combination of ABBV-CLS-7262, for use in treating a subject afflicted with ALS as an add-on therapy to pridopidine.

In an embodiment, the pharmaceutical composition comprises an amount of a compound which is riluzole for use in treating a subject afflicted with ALS simultaneously or contemporaneously with pridopidine or pharmaceutically acceptable salt thereof. In another embodiment the pharmaceutical composition comprises an amount of a compound which is edaravone for use in treating a subject afflicted with ALS simultaneously or contemporaneously with pridopidine or pharmaceutically acceptable salt thereof. In another embodiment the pharmaceutical composition comprises an amount of a compound which is dextromethorphan/quinidine for use in treating a subject afflicted with ALS simultaneously or contemporaneously with pridopidine or pharmaceutically acceptable salt thereof. In another embodiment the pharmaceutical composition comprises an amount of a compound which is sodium phenylbutyrate (PB) for use in treating a subject afflicted with ALS simultaneously or contemporaneously with pridopidine or pharmaceutically acceptable salt thereof. In another embodiment the pharmaceutical composition comprises an amount of a compound which is tauroursodeoxycholic acid for use in treating a subject afflicted with ALS simultaneously or contemporaneously with pridopidine or pharmaceutically acceptable salt thereof. In another embodiment the pharmaceutical composition comprises an amount of combination of sodium phenylbutyrate (PB)/tauroursodeoxycholic acid (i.e. AMX0035) for use in treating a subject afflicted with ALS simultaneously or contemporaneously with pridopidine or pharmaceutically acceptable salt thereof. In another embodiment the pharmaceutical composition comprises an amount of combination of SLS-005 (Trehalose), for use in treating a subject afflicted with ALS simultaneously or contemporaneously with pridopidine or pharmaceutically acceptable salt thereof. In another embodiment the pharmaceutical composition comprises an amount of combination of DNL343, for use in treating a subject afflicted with ALS simultaneously or contemporaneously with pridopidine or pharmaceutically acceptable salt thereof. In another embodiment the pharmaceutical composition comprises an amount of CNM-Au8 nanocrystalline gold for use in treating a subject afflicted with ALS simultaneously or contemporaneously with pridopidine or pharmaceutically acceptable salt thereof. In another embodiment, the pharmaceutical composition comprises an amount of combination of, ABBV-CLS-7262 for use in treating a subject afflicted with ALS simultaneously or contemporaneously with pridopidine or pharmaceutically acceptable salt thereof.

The invention also provides a compound which is riluzole for use as an add-on therapy to pridopidine in treating a subject afflicted with ALS.

The invention also provides a compound which is edaravone for use as an add-on therapy to pridopidine in treating a subject afflicted with ALS.

The invention also provides a compound which is dextromethorphan/quinidine for use as an add-on therapy to pridopidine in treating a subject afflicted with ALS.

The invention also provides a compound which is sodium phenylbutyrate (PB) for use as an add-on therapy to pridopidine in treating a subject afflicted with ALS.

The invention also provides a compound which is tauroursodeoxycholic acid for use as an add-on therapy to pridopidine in treating a subject afflicted with ALS.

The invention also provides a compound which is SLS-005 (Trehalose) for use as an add-on therapy to pridopidine in treating a subject afflicted with ALS.

The invention also provides a compound which is DNL343 for use as an add-on therapy to pridopidine in treating a subject afflicted with ALS.

The invention also provides a compound which is CNM-Au8 nanocrystalline gold for use as an add-on therapy to pridopidine in treating a subject afflicted with ALS.

The invention also provides a compound which is ABBV-CLS-7262 for use as an add-on therapy to pridopidine in treating a subject afflicted with ALS.

The invention also provides a combination of sodium phenylbutyrate (PB)/tauroursodeoxycholic acid (i.e. AMX0035) for use as an add-on therapy to pridopidine in treating a subject afflicted with ALS.

The invention also provides pridopidine or pharmaceutically acceptable salt thereof for use as an add-on therapy to a compound which is riluzole in treating a subject afflicted with ALS.

The invention also provides pridopidine or pharmaceutically acceptable salt for use as an add-on therapy to a compound which is edaravone in treating a subject afflicted with ALS.

The invention also provides pridopidine or pharmaceutically acceptable salt for use as an add-on therapy to a compound which is dextromethorphan/quinidine in treating a subject afflicted with ALS.

The invention also provides pridopidine or pharmaceutically acceptable salt for use as an add-on therapy to a compound which is laquinimod in treating a subject afflicted with ALS.

The invention also provides pridopidine or pharmaceutically acceptable salt for use as an add-on therapy to a compound which is sodium phenylbutyrate (PB) in treating a subject afflicted with ALS.

The invention also provides pridopidine or pharmaceutically acceptable salt for use as an add-on therapy to a compound which is tauroursodeoxycholic acid in treating a subject afflicted with ALS.

The invention also provides pridopidine or pharmaceutically acceptable salt for use as an add-on therapy to a combination of sodium phenylbutyrate (PB)/tauroursodeoxycholic acid (i.e. AMX0035) in treating a subject afflicted with ALS.

The invention also provides pridopidine or pharmaceutically acceptable salt for use as an add-on therapy to a compound which is SLS-005 (Trehalose) in treating a subject afflicted with ALS.

The invention also provides pridopidine or pharmaceutically acceptable salt for use as an add-on therapy to a compound which is DNL343 in treating a subject afflicted with ALS.

The invention also provides pridopidine or pharmaceutically acceptable salt thereof for use as an add-on therapy to a compound which is CNM-Au8 nanocrystalline gold in treating a subject afflicted with ALS.

The invention also provides pridopidine or pharmaceutically acceptable salt thereof for use as an add-on therapy to a compound which is ABBV-CLS-7262 in treating a subject afflicted with ALS. In an embodiment the add-on therapy is for the treatment, prevention, or alleviation of a symptom of ALS.

The invention also provides a combination of a compound which is riluzole and pridopidine or pharmaceutically acceptable salt thereof for use in the treatment, prevention, or alleviation of a symptom of ALS.

The invention also provides a combination of a compound which is edaravone and pridopidine or pharmaceutically acceptable salt thereof for use in the treatment, prevention, or alleviation of a symptom of ALS.

The invention also provides a combination of a compound which is dextromethorphan/quinidine and pridopidine or pharmaceutically acceptable salt thereof for use in the treatment, prevention, or alleviation of a symptom of ALS.

The invention also provides a combination of a compound which is sodium phenylbutyrate (PB) and pridopidine or pharmaceutically acceptable salt thereof for use in the treatment, prevention, or alleviation of a symptom of ALS.

The invention also provides a combination of a compound which is tauroursodeoxycholic acid and pridopidine or pharmaceutically acceptable salt thereof for use in the treatment, prevention, or alleviation of a symptom of ALS.

The invention also provides a combination of sodium phenylbutyrate (PB)/tauroursodeoxycholic acid (i.e. AMX0035) and pridopidine or pharmaceutically acceptable salt thereof for use in the treatment, prevention, or alleviation of a symptom of ALS.

The invention also provides a combination of SLS-005 (Trehalose) and pridopidine or pharmaceutically acceptable salt thereof for use in the treatment, prevention, or alleviation of a symptom of ALS.

The invention also provides for a combination DNL343 and pridopidine or pharmaceutically acceptable salt thereof for use in the treatment, prevention, or alleviation of a symptom of ALS.

The invention also provides for a combination of Au8 nanocrystalline gold and pridopidine or pharmaceutically acceptable salt thereof for use in the treatment, prevention, or alleviation of a symptom of ALS.

The invention also provides for a combination ABBV-CLS-7262 and pridopidine or pharmaceutically acceptable salt thereof for use in the treatment, prevention, or alleviation of a symptom of ALS.

The method, use and composition further include decreasing the rate of neurological deterioration in the subject.

In an embodiment, the methods of the present invention further comprise administering to the subject a therapeutically effective amount of a Second compound which is riluzole or edaravone. In an embodiment, the methods of the present invention further comprise administering to the subject a therapeutically effective amount of a Second compound which is dextromethorphan/quinidine. In an embodiment, the methods of the present invention further comprise administering to the subject a therapeutically effective amount of a Second compound which is sodium phenylbutyrate (PB), or tauroursodeoxycholic acid. In an embodiment, the methods of the present invention further comprise administering to the subject a therapeutically effective amount of a combination of sodium phenylbutyrate (PB)/tauroursodeoxycholic acid (i.e. AMX0035). In an embodiment, the methods of the present invention further comprise administering to the subject a therapeutically effective amount of a Second compound which is SLS-005 (Trehalose). In an embodiment, the methods of the present invention further comprise administering to the subject a therapeutically effective amount of a Second compound which is DNL343.

In an embodiment, the methods of the present invention further comprise administering to the subject a therapeutically effective amount of a Second compound which is Au8 nanocrystalline gold. In an embodiment, the methods of the present invention further comprise administering to the subject a therapeutically effective amount of a Second compound which is ABBV-CLS-7262. In an embodiment, the Second compound is riluzole. In another embodiment, the Second compound is edaravone. In another embodiment, the Second compound is dextromethorphan/quinidine. In another embodiment, the Second compound is laquinimod. In another embodiment, the Second compound is sodium phenylbutyrate (PB), or tauroursodeoxycholic acid. In an embodiment, the Second compound is SLS-005 (Trehalose). In an embodiment, the Second compound is DNL343. In an embodiment, the Second compound is Au8 nanocrystalline gold. In an embodiment, the Second compound is ABBV-CLS-7262.

In an embodiment of the invention, pridopidine or pharmaceutically acceptable salt thereof and the Second compound are administered in one unit. In another embodiment the pridopidine and the Second compound are administered in more than one unit.

In an embodiment, the amount of pridopidine and the amount of the Second compound are administered simultaneously. In an embodiment, the amount of pridopidine and the amount of the Second compound are administered contemporaneously.

In another embodiment, the administration of the Second compound precedes the administration of pridopidine or pharmaceutically acceptable salt thereof. In another embodiment, the administration of pridopidine or pharmaceutically acceptable salt thereof precedes the administration of the Second compound.

In an embodiment, a subject is receiving edaravone therapy prior to initiating pridopidine therapy. In another embodiment, a subject is receiving riluzole prior to initiating pridopidine therapy. In another embodiment, a subject is receiving laquinimod prior to initiating pridopidine therapy. In another embodiment, a subject is receiving dextromethorphan/quinidine prior to initiating pridopidine therapy. In another embodiment, a subject is receiving sodium phenylbutyrate (PB) prior to initiating pridopidine therapy. In another embodiment, a subject is receiving tauroursodeoxycholic acid prior to initiating pridopidine therapy. In another embodiment, a subject is receiving a combination of sodium phenylbutyrate (PB)/tauroursodeoxycholic acid (i.e. AMX0035) prior to initiating pridopidine therapy.

In an embodiment, a subject is receiving SLS-005 (Trehalose) therapy prior to initiating pridopidine therapy. In an embodiment, a subject is receiving DNL343 therapy prior to initiating pridopidine therapy.

In an embodiment, a subject is receiving CNM-Au8 nanocrystalline gold therapy prior to initiating pridopidine therapy. In an embodiment, a subject is receiving ABBV-CLS-7262 therapy prior to initiating pridopidine therapy.

In another embodiment, a subject is receiving edaravone therapy for at least 24 weeks, 28 weeks, 48 weeks, or 52 weeks prior to initiating pridopidine therapy. In another embodiment, a subject is receiving riluzole therapy for at least 24 weeks, 28 weeks, 48 weeks, or 52 weeks prior to initiating pridopidine therapy. In another embodiment, a subject is receiving dextromethorphan/quinidine therapy for at least 1 week, 2 weeks, 4 weeks, or 6 weeks prior to initiating pridopidine therapy. In another embodiment, a subject is receiving sodium phenylbutyrate (PB) therapy for at least 1 week, 2 weeks, 4 weeks, or 6 weeks prior to initiating pridopidine therapy. In another embodiment, a subject is receiving tauroursodeoxycholic acid therapy for at least 1 week, 2 weeks, 4 weeks, or 6 weeks prior to initiating pridopidine therapy. In another embodiment, a subject is receiving combination of sodium phenylbutyrate (PB)/tauroursodeoxycholic acid (i.e. AMX0035) therapy for at least 1 week, 2 weeks, 4 weeks, or 6 weeks prior to initiating pridopidine therapy. In another embodiment, a subject is receiving SLS-005 (Trehalose) therapy for at least 24 weeks, 28 weeks, 48 weeks, or 52 weeks prior to initiating pridopidine therapy. In another embodiment, a subject is receiving DNL343 therapy for at least 24 weeks, 28 weeks, 48 weeks, or 52 weeks prior to initiating pridopidine therapy. In another embodiment, a subject is receiving CNM-Au8 nanocrystalline gold therapy for at least 24 weeks, 28 weeks, 48 weeks, or 52 weeks prior to initiating pridopidine therapy. In another embodiment, a subject is receiving ABBV-CLS-7262 therapy for at least 24 weeks, 28 weeks, 48 weeks, or 52 weeks prior to initiating pridopidine therapy. In an embodiment, a subject is receiving pridopidine therapy prior to initiating edaravone therapy. In another embodiment, a subject is receiving pridopidine therapy for at least 24 weeks, 28 weeks, 48 weeks, or 52 weeks prior to initiating edaravone therapy.

In an embodiment, a subject is receiving pridopidine therapy prior to initiating riluzole therapy. In another embodiment, a subject is receiving pridopidine therapy for at least 24 weeks, 28 weeks, 48 weeks, or 52 weeks prior to initiating riluzole therapy.

In an embodiment, a subject is receiving pridopidine therapy prior to initiating laquinimod therapy. In another embodiment, a subject is receiving pridopidine therapy for at least 24 weeks, 28 weeks, 48 weeks, or 52 weeks prior to initiating laquinimod therapy.

In an embodiment, a subject is receiving pridopidine therapy prior to initiating dextromethorphan/quinidine therapy. In another embodiment, a subject is receiving pridopidine therapy for at least 24 weeks, 28 weeks, 48 weeks, or 52 weeks prior to initiating dextromethorphan/quinidine therapy.

In an embodiment, a subject is receiving pridopidine therapy prior to initiating sodium phenylbutyrate (PB), tauroursodeoxycholic acid or combination of sodium phenylbutyrate (PB)/tauroursodeoxycholic acid (i.e. AMX0035) therapy. In another embodiment, a subject is receiving pridopidine therapy for at least 24 weeks, 28 weeks, 48 weeks, or 52 weeks prior to initiating sodium phenylbutyrate (PB), tauroursodeoxycholic acid or combination of sodium phenylbutyrate (PB)/tauroursodeoxycholic acid (i.e. AMX0035) therapy.

In an embodiment, a subject is receiving pridopidine therapy prior to initiating SLS-005 (Trehalose) therapy. In another embodiment, a subject is receiving pridopidine therapy for at least 24 weeks, 28 weeks, 48 weeks, or 52 weeks prior to SLS-005 (Trehalose) therapy.

In an embodiment, a subject is receiving pridopidine therapy prior to initiating DNL343. In another embodiment, a subject is receiving pridopidine therapy for at least 24 weeks, 28 weeks, 48 weeks, or 52 weeks prior to initiating DNL343 therapy.

In an embodiment, a subject is receiving pridopidine therapy prior to initiating CNM-Au8 nanocrystalline gold therapy. In another embodiment, a subject is receiving pridopidine therapy for at least 24 weeks, 28 weeks, 48 weeks, or 52 weeks prior to initiating CNM-Au8 nanocrystalline gold therapy.

In an embodiment, a subject is receiving pridopidine therapy prior to initiating ABBV-CLS-7262 therapy. In another embodiment, a subject is receiving pridopidine therapy for at least 24 weeks, 28 weeks, 48 weeks, or 52 weeks prior to initiating ABBV-CLS-7262 therapy.

In an embodiment, between 0.5 mg to 1.5 mg laquinimod is administered to the patient per day

In another embodiment, 0.5 mg, or 1.0 mg laquinimod is administered to the patient per day. In an embodiment, laquinimod is administered orally.

In an embodiment, between 10-200 mg riluzole is administered to the patient per day. In another embodiment, 50 mg, 100 mg, or 200 mg riluzole is administered to the patient per day.

In an embodiment, riluzole is administered orally. In an embodiment dextromethorphan/quinidine is administered orally.

In an embodiment sodium phenylbutyrate (PB) is administered orally. In another embodiment, sodium phenylbutyrate (PB) between 1-10 gr/day is administered to the patient per day. In another embodiment, between 1-5 gr/day, 1-3 gr/day, 4-10 gr/day. In another embodiment, sodium phenylbutyrate (PB) is administered once a day, twice a day or more than twice a day.

In an embodiment tauroursodeoxycholic acid is administered orally. In another embodiment, tauroursodeoxycholic acid between 0.5-3 gr/day is administered to the patient per day. In another embodiment, between 0.5-2 gr/day, 1-3 gr/day. In another embodiment, tauroursodeoxycholic acid is administered once a day, twice a day or more than twice a day.

In an embodiment, AMX0035 is administered orally and is administered to the patient in a therapeutic combination including between 0.5-5 g of sodium phenylbutyrate and between 0.2-5 gr/day of tauroursodeoxycholic acid (TUDCA). In another embodiment 3 gr/day of sodium phenylbutyrate and 1 gr/day tauroursodeoxycholic acid (TUDCA) per day, or 9 gr/day of sodium phenylbutyrate and 2 gr/day tauroursodeoxycholic acid (TUDCA) per day. In another embodiment, in a combination including between 1-10 gr/day sodium phenylbutyrate and between 0.5-3 gr/day of tauroursodeoxycholic acid. In another embodiment, AMX0035 is administered once a day, twice a day or more than twice a day.

In an embodiment, between 5-60 mg edaravone is administered to the patient per day. In another embodiment, 30 mg, or 60 mg edaravone is administered to the patient per day.

In an embodiment, edaravone is administered by intravenous infusion. In another embodiment, edaravone is administered once per day for 10 days followed by a 14-day drug-free period. In another embodiment, edaravone is administered once per day for 14 days followed by a 14-day drug-free period.

In an embodiment SLS-005 (Trehalose) is administered by intravenously. In another embodiment, SLS-005 (Trehalose) is administered in a weekly dose of between 0.05-1 g/kg/weekly. In another embodiment SLS-005 (Trehalose) is administered in a weekly dose of between 0.1-0.5 g/kg/week, 0.25-0.75 g/kg/week or 0.6-1 g/kg/week.

In an embodiment DNL343 is administered orally. In another embodiment, DNL343 is administered in a daily dose. In another embodiment, DNL343 is administered once a day, twice a day or more than twice a day.

In an embodiment CNM-Au8 nanocrystalline gold is administered orally. In another embodiment, CNM-Au8 nanocrystalline gold is administered in a daily dose between 5-50 mg/day. In another embodiment CNM-Au8 nanocrystalline gold is administered in a daily dose of between 5-10 mg/day, 15-20 mg/day, 15-30 mg/day, 20-30 mg/day. In another embodiment, CNM-Au8 nanocrystalline gold is administered once a day, twice a day or more than twice a day.

In an embodiment ABBV-CLS-7262 is administered orally. In another embodiment, ABBV-CLS-7262 is administered in a daily dose. In another embodiment, ABBV-CLS-7262 is administered once a day, twice a day or more than twice a day.

In an embodiment, each of the amount of the Second compound when taken alone, and the amount of pridopidine when taken alone is effective to treat a subject. In another embodiment, either the amount of the Second compound when taken alone, or the amount of pridopidine when taken alone, is less effective to treat the subject. In another embodiment, either the amount of the Second compound when taken alone, or the amount of pridopidine when taken alone, is not effective to treat the subject.

In an embodiment, pridopidine is administered adjunctively to the Second compound. In another embodiment, the Second compound is administered adjunctively to pridopidine.

In an embodiment, a loading dose of an amount different from the intended dose is administered for a period of time at the start of the periodic administration.

In some embodiments the methods of this invention make use of a pharmaceutical composition comprising pridopidine or pharmaceutically acceptable salt thereof and at least one analog Compounds 1-7 or pharmaceutically acceptable salt thereof.

In other embodiments the methods provided herein make use of a pharmaceutical composition comprising pridopidine or pharmaceutically acceptable salt thereof and compound 1 or pharmaceutically acceptable salt thereof. In other embodiments this invention provides a pharmaceutical composition comprising pridopidine or pharmaceutically acceptable salt thereof and compound 2 or pharmaceutically acceptable salt thereof. In other embodiments, this invention provides a pharmaceutical composition comprising pridopidine or pharmaceutically acceptable salt thereof and compound 3 or pharmaceutically acceptable salt thereof. In other embodiments this invention provides a pharmaceutical composition comprising pridopidine or pharmaceutically acceptable salt thereof and compound 4 or pharmaceutically acceptable salt thereof. In other embodiments this invention provides a pharmaceutical composition comprising pridopidine or pharmaceutically acceptable salt thereof and compound 5 or pharmaceutically acceptable salt thereof. In other embodiments this invention provides a pharmaceutical composition comprising pridopidine or pharmaceutically acceptable salt thereof and compound 6 or pharmaceutically acceptable salt thereof. In other embodiments this invention provides a pharmaceutical composition comprising pridopidine or pharmaceutically acceptable salt thereof and compound 7 or pharmaceutically acceptable salt thereof. In other embodiments this invention provides a pharmaceutical composition comprising pridopidine or pharmaceutically acceptable salt thereof and compound 1 and compound 4 or pharmaceutically acceptable salt thereof. In other embodiments, the concentration of compounds 1, 2, 3, 4, 5, 6 or 7 or pharmaceutically acceptable salt thereof within the composition is between 0.001% w/w to 10% w/w. In other embodiments, the concentration of compounds 1, 2, 3, 4, 5, 6 or 7 or pharmaceutically acceptable salt thereof within the composition is between 0.001% w/w to 0.05% w/w. In other embodiments, the concentration of compounds 1, 2, 3, 4, 5, 6 or 7 or pharmaceutically acceptable salt thereof within the composition is between 0.001% w/w to 0.5% w/w. In other embodiments, the concentration of compounds 1, 2, 3, 4, 5, 6 or 7 or pharmaceutically acceptable salt thereof within the composition is between 0.001% w/w to 0.15% w/w. In other embodiments, the concentration of compounds 1, 2, 3, 4, 5, 6 or 7 or pharmaceutically acceptable salt thereof within the composition is between 0.01% w/w to 0.15% w/w. In other embodiments, the concentration of compounds 1, 2, 3, 4, 5, 6 or 7 or pharmaceutically acceptable salt thereof within the composition is between 0.01% w/w to 0.35% w/w. In other embodiments, the concentration of compounds 1, 2, 3, 4, 5, 6 or 7 or pharmaceutically acceptable salt thereof within the composition is between 0.01% w/w to 1% w/w.

In an embodiment provided is a method of enhancing BDNF axonal transport in motor neurons in a subject afflicted with ALS comprising administering to the subject an amount of pridopidine or pharmaceutically acceptable salt thereof effective to enhance BDNF axonal transport in the subject's motor neurons.

In an embodiment provided is a method of enhancing ERK activation in motor neurons of a subject afflicted with ALS comprising administering to the subject an amount of pridopidine or pharmaceutically acceptable salt thereof effective to enhance ERK activation in the subject's motor neurons.

In an embodiment provided is a method of preserving neuromuscular junction (NMJ) structure in muscle cells of a subject afflicted with ALS comprising administering to the subject an amount of pridopidine or pharmaceutically acceptable salt thereof effective to preserving neuromuscular junction structure in the subject's muscles.

Further provided is a method of improving muscle contraction in a subject afflicted with ALS comprising administering to the subject an amount of pridopidine or pharmaceutically acceptable salt thereof effective to improve muscle contraction function in the subject.

Further provided is a method of improving innervation rate of muscle tissue in a subject afflicted with ALS comprising administering to the subject an amount of pridopidine or pharmaceutically acceptable salt thereof effective to improve the innervation rate in the subject.

In an embodiment, provided is a method of enhancing motor neuron axonal growth in a subject afflicted with ALS comprising administering to the subject an amount of pridopidine or pharmaceutically acceptable salt thereof effective to enhance motor neuron axonal growth in the subject.

In an embodiment, provided is a method of enhancing muscle cell survival in a subject afflicted with ALS comprising administering to the subject an amount of pridopidine or pharmaceutically acceptable salt thereof effective to enhancing muscle cell survival in the subject.

In an embodiment, provided is a method of reducing progression of muscle fiber wasting in a subject afflicted with ALS comprising administering to the subject an amount of pridopidine or pharmaceutically acceptable salt thereof effective to reduce progression of muscle fiber wasting in the subject.

In an embodiment, provided is a method of reducing axonal degeneration in a subject afflicted with ALS comprising administering to the subject an amount of pridopidine or pharmaceutically acceptable salt thereof effective to reduce axonal degeneration in the subject.

In an embodiment, provided is a method of preserving NMJ formation in a subject afflicted with ALS comprising administering to the subject an amount of pridopidine or pharmaceutically acceptable salt thereof effective to preserve NMJ formation in the subject.

In an embodiment, provided is a method of preserving NMJ structure and function in a subject afflicted with ALS comprising administering to the subject an amount of pridopidine or pharmaceutically acceptable salt thereof effective to preserve NMJ structure and function in the subject.

In an embodiment, provided is a method of reducing protein aggregation in a subject afflicted with ALS comprising administering to the subject an amount of pridopidine or pharmaceutically acceptable salt thereof effective to reduce protein aggregation in the subject.

In an embodiment, provided is a method of attenuating pseudobulbar disease progression in a subject afflicted with ALS comprising administering to the subject an amount of pridopidine or pharmaceutically acceptable salt thereof effective to attenuate pseudobulbar disease progression in the subject.

### Pharmaceutically Acceptable Salts

As used herein, “pridopidine” means pridopidine base or a pharmaceutically acceptable salt thereof, as well as derivatives, for example deuterium-enriched version of pridopidine and salts. Examples of deuterium-enriched pridopidine and salts and their methods of preparation may be found in U.S. Application Publication Nos. 2013-0197031, 2016-0166559 and 2016-0095847, the entire content of each of which is hereby incorporated by reference. In certain embodiments, pridopidine is a pharmaceutically acceptable salt, such as the HCl salt or tartrate salt. Preferably, in any embodiments of the invention as described herein, the pridopidine is in the form of its hydrochloride salt.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methane sulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

“Deuterium-enriched” means that the abundance of deuterium at any relevant site of the compound is more than the abundance of deuterium naturally occurring at that site in an amount of the compound. The naturally occurring distribution of deuterium is about 0.0156%. Thus, in a “deuterium-enriched” compound, the abundance of deuterium at any of its relevant sites is more than 0.0156% and can range from more than 0.0156% to 100%. Deuterium-enriched compounds may be obtained by exchanging hydrogen with deuterium or synthesizing the compound with deuterium-enriched starting materials.

### Pharmaceutical Compositions

While the pridopidine for use according to the invention may be administered in the form of the raw compound, preferred administration of pridopidine, optionally in the form of a physiologically acceptable salt, is in a pharmaceutical composition together with one or more adjuvants, excipients, carriers, buffers, diluents, and/or other customary pharmaceutical auxiliaries.

In an embodiment, the invention provides pharmaceutical compositions comprising the pridopidine or pharmaceutically acceptable salts or derivatives thereof, together with one or more pharmaceutically acceptable carriers therefore, and, optionally, other therapeutic and/or prophylactic ingredients known and used in the art including, but not limited to, riluzole, edaravone Nuedexta® (dextromethorphan/quinidine), sodium phenylbutyrate (PB), tauroursodeoxycholic acid, a combination of sodium phenylbutyrate (PB)/tauroursodeoxycholic acid (i.e. AMX0035), SLS-005 (Trehalose), DNL343, CNM-Au8 nanocrystalline gold or ABBV-CLS-7262.

In an embodiment, the invention provides pharmaceutical compositions comprising the pridopidine or pharmaceutically acceptable salts or derivatives thereof, together with at least one of pridopidine's analog of Compounds 1-7 or pharmaceutically acceptable salt thereof.

In an embodiment, the invention provides pharmaceutical compositions comprising at least one of Compounds 1-7. In other embodiments, the composition comprises Compound 1 or pharmaceutically acceptable salt thereof. In other embodiments, the composition comprises Compound 2 or pharmaceutically acceptable salt thereof. In other embodiments, the composition comprises Compound 3 or pharmaceutically acceptable salt thereof. In other embodiments, the composition comprises Compound 4 or pharmaceutically acceptable salt thereof. In other embodiments, the composition comprises Compound 5 or pharmaceutically acceptable salt thereof. In other embodiments, the composition comprises Compound 6 or pharmaceutically acceptable salt thereof. In other embodiments, the composition comprises Compound 7 or pharmaceutically acceptable salt thereof. In other embodiments, the composition comprises Compound 1 and Compound 4 or pharmaceutically acceptable salt thereof.

The carrier(s) must be “acceptable” in the sense of being compatible with the other ingredients of the formulation and suitable for administration to a human subject.

### Combination Therapy

When the invention comprises a combination of the active compound and an additional one, or more, therapeutic and/or prophylactic ingredients, the combination of the invention may be formulated for its simultaneous or contemporaneous administration, with at least a pharmaceutically acceptable carrier, additive, adjuvant, or vehicle. This has the implication that the combination of the two active compounds may be administered:
as a combination that is part of the same medicament formulation, the two active compounds being then administered simultaneously, or
as a combination of two units, each with one of the active substances giving rise to the possibility of simultaneous or contemporaneous administration.

### Administration

The pharmaceutical composition of the invention may be administered by any convenient route, which suits the desired therapy. Preferred routes of administration include oral administration, in particular in tablet, in capsule, in dragée, in powder, suspension or in liquid form, intranasal administration, intradermal administration, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Tablets may contain suitable binders, lubricants, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose, and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc, and the like. Disintegrators (disintegrants) include, without limitation, starch, methyl cellulose, agar, bentonite, xanthan gum, croscarmellose sodium, sodium starch glycolate and the like.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds); Modern Pharmaceutics Drugs and the Pharmaceutical Sciences, Vol. 40 (Gilbert S. Banker, Christopher T. Rhodes, Eds). These references in their entireties are hereby incorporated by reference into this application.

### Terms

As used herein, and unless stated otherwise, each of the following terms shall have the definition set forth below.

As used herein, “riluzole” means riluzole or a pharmaceutically acceptable salt thereof, as well as derivatives, for example deuterium-enriched version of riluzole and salts. Riluzole is described in Prescribers' Digital Reference which is hereby incorporated by reference (Riluzole PDR 2017).

As used herein, “edaravone” means edaravone or a pharmaceutically acceptable salt thereof, as well as derivatives, for example deuterium-enriched version of edaravone and salts. Edaravone is described in Prescribers' Digital Reference which is hereby incorporated by reference (Edaravone PDR 2017).

As used herein, “AMX0035” means an oral combination of two drugs already in use, sodium phenylbutyrate (PB) and tauroursodeoxycholic acid (TUDCA). AMX0035 is a combination therapy designed to reduce neuronal death through blockade of key cellular death pathways originating in the mitochondria and endoplasmic reticulum (ER).

A “combination of dextromethorphan and quinidine” or “dextromethorphan/quinidine” or “dextromethorphan hydrobromide/quinidine sulfate” refers to a combination of dextromethorphan hydrobromide (20 mg) and quinidine sulfate (10 mg) such as Nuedexta®. Nuedexta® is a drug currently on the market for treating pseudobulbar affect (PBA) in, inter alia, ALS patients. Nuedexta® has been shown to act on sigma-1 and NMDA receptors in the brain. Recent data demonstrate that the combination has an effect on bulbar function in ALS, but not on other aspects of motor functions (Smith 2017).

Dextromethorphan hydrobromide/quinidine sulfate is described in Prescribers' Digital Reference which is hereby incorporated by reference (Dextromethorphan hydrobromide/quinidine sulfate PDR 2017).

Sodium Phenylbutyrate (PB)—Sodium Phenylbutyrate is the sodium salt of phenylbutyrate, a derivative of the short-chain fatty acid butyrate, with potential antineoplastic activity. Phenylbutyrate reversibly inhibits class I and II histone deacetylases (HDACs), which may result in a global increase in gene expression, decreased cellular proliferation, increased cell differentiation, and the induction of apoptosis in susceptible tumor cell populations.

Tauroursodeoxycholic acid (TUDCA/TURSO)—Tauroursodeoxycholic acid is a bile acid taurine conjugate derived from ursodeoxycholic acid. It has a role as a human metabolite, an anti-inflammatory agent, a neuroprotective agent, an apoptosis inhibitor, a cardioprotective agent and a bone density conservation agent. It derives from an ursodeoxycholic acid. It is a conjugate acid of a tauroursodeoxycholate

CNM-Au8 nanocrystalline gold are small nanocrystals that provide energetic assistance by supporting bioenergetic reactions and eliminating harmful bioproducts of cell metabolism. CNM-Au8 shows neuroprotective effects in preclinical models. CNM-Au8 consists solely of gold nanoparticles, composed of clean-surfaced, faceted, geometrical crystals held in suspension in sodium bicarbonate buffered, pharmaceutical grade water.

Trehalose (SLS-005) is a low molecular weight disaccharide ((2R,3S,4S,5R,6R)-2-(hydroxymethyl)-6-[(2R,3R,4S,5S,6R)-3,4,5-trihydroxy-6-(hydroxymethyl) oxan-2-yl]oxyoxane-3,4,5-triol) that stabilizes protein and activates autophagy, the process that clears waste materials from cells. Trehalose (SLS-005) activates transcription factor EB, which is key to the expression of autophagy-related genes.

DNL343 is an investigational, orally administered activator of the eukaryotic initiation factor EIF2b. It inhibits the cell's unfolded protein response, part of the cellular stress response, in an attempt to restore protein synthesis.

ABBV-CLS-7262 is an investigational, orally administered activator of the eukaryotic initiation factor EIF2b. The molecule is an integrated stress response (ISR) inhibitor, also known as ISRIB.

As used herein, an “amount” or “dose” of pridopidine as measured in milligrams refers to the milligrams of underivatized pridopidine base present in a preparation, regardless of the form of the preparation. A “dose of 45 mg pridopidine” means the amount of pridopidine in a preparation is sufficient to provide 45 mg of underivatized pridopidine base having a naturally occurring isotope distribution, regardless of the form of the preparation. Thus, when in the form of a salt, e.g., a pridopidine hydrochloride, the mass of the salt form necessary to provide a dose of 45 mg underivatized pridopidine base would be greater than 45 mg due to the presence of the additional salt ion. Similarly, when in the form of a deuterium-enriched derivative, the mass of the derivatized form necessary to provide a dose of 45 mg underivatized pridopidine base having a naturally occurring isotope distribution would be greater than 45 mg due to the presence of the additional deuterium.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1; 0.2 . . . 0.9; and 1, 2 . . . 49 mg unit amounts are included as embodiments of this invention. By any range of time disclosed herein (i.e. weeks, months, or years), it is meant that all lengths of time of days and/or weeks within the range are specifically disclosed as part of the invention. Thus, for example, 3-6 months means that 3 months and 1 day, 3 months and 1 week, and 4 months are included as embodiments of the invention.

As used herein, “about” in the context of a numerical value or range means±10% of the numerical value or range recited or claimed.

As used herein, “monotherapy” means treatment with a single active agent, for example treatment with pridopidine alone.

As used herein, “adjunctively” means treatment with or administration of an additional compound (second compound), with a primary compound, for example for increasing the efficacy or safety of the primary compound or for facilitating its activity.

As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times a week and so on, etc.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the pridopidine and a second compound (for example, riluzole). In this case, the combination may be the admixture or separate containers of the pridopidine and the second compound that are combined just prior to administration. Contemporaneous administration, or concomitant administration refer to the separate administration of the pridopidine and the second compound (for example, riluzole) at the same time, or at times sufficiently close together that an additive or preferably synergistic activity relative to the activity of either the pridopidine or the second compound alone is observed or in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding pridopidine therapy to a patient already receiving riluzole therapy.

As used herein, “effective” when referring to an amount of pridopidine refers to the quantity of pridopidine that is sufficient to yield a desired therapeutic response. In a preferred embodiment, the quantity of pridopidine administered does not result in adverse side-effects (such as toxicity, irritation, or allergic response).

“Administering to the subject” or “administering to the (human) patient” means the giving of, dispensing of, or application of medicines, drugs, or remedies to a subject/patient to relieve, cure, or reduce the symptoms associated with a disease, disorder, or condition, e.g., a pathological condition.

“Treating” as used herein encompasses inducing inhibition, regression, or stasis of a disease or disorder, or lessening, suppressing, inhibiting, reducing the severity of, eliminating, or substantially eliminating, or ameliorating a symptom of the disease or disorder.

“Inhibition” of disease progression or disease complication in a subject means preventing or reducing the disease progression and/or disease complication in the subject.

A “symptom” associated with a disease or disorder includes any clinical or laboratory manifestation associated with the disease or disorder and is not limited to what the subject can feel or observe.

As used herein, “a subject afflicted with” a disease, disorder or condition means a subject who has been clinically diagnosed to have the disease, disorder, or condition.

Glial cell-derived neurotrophic factor (GDNF) is a protein encoded by the GDNF gene and is believed to promote the survival of many types of neurons them.

Brain-derived neurotrophic factor (BDNF) is a protein produced by neurons and serves to keep functioning and to promote the growth of neurons and neurogenesis.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

All combinations, sub-combinations, and permutations of the various elements of the methods and uses described herein are envisaged and are within the scope of the invention.

The following numbered clauses define various aspects and features of the present invention:

1. A method for treating a subject afflicted with amyotrophic lateral sclerosis (ALS), comprising periodically administering to the subject a composition comprising an amount of pridopidine effective to treat the subject.

2. The method of clause 1, wherein the amount of pridopidine is effective to improve, maintain or lessen the decline of a symptom of the ALS in the subject.

3. The method of clause 2, where in the symptom is muscles stiffness, muscle weakness, muscle wasting, muscle cramps, difficulty speaking, difficulty swallowing, difficulty breathing, difficulty chewing, difficulty walking, fasciculations, and/or worsening posture.

4. The method of clause 1 and clause 2, wherein the amount of pridopidine is effective to reduce, maintain or lessen the increase in Neurofilament Light (NfL) protein levels.

5. The method of any one of clauses 1-4, wherein the ALS is sporadic ALS.

6. The method of any one of clauses 1-5, wherein the amount of pridopidine is administered daily or wherein the amount of pridopidine is administered more often than once daily.

7. The method of any one of clauses 1-5, wherein the amount of pridopidine is administered twice daily.

8. The method of any one of clauses 1-5, wherein the amount of pridopidine is administered less often than once daily.

9. The method of any one of clauses 1-6, wherein the amount of pridopidine is administered orally.

10. The method of any one of clauses 1-7, wherein the amount of pridopidine administered is from 22.5 mg per day to 225 mg per day.

11. The method of clause 10, wherein the amount of pridopidine administered is from 45 mg per day to 180 mg per day.

12. The method of clause 10, wherein the amount of pridopidine administered is 5 mg, 10 mg, 22.5 mg, 45 mg, 67.5, mg, 90 mg, 100 mg, 112.5 mg, 125 mg, 135 mg, 150 mg, or 180 mg per day.

13. The method of any one of clauses 1-12, wherein the periodic administration continues for at least 24 weeks.

14. The method of any one of clauses 1-13, wherein the pridopidine is pridopidine hydrochloride.

15. The method of any one of clauses 1-14, wherein the subject is a human subject.

16. The method of any one of clauses 1-15, further comprising administering to the subject a therapeutically effective amount of a second compound.

17. The method of any one of clauses 1-16, wherein the composition comprising pridopidine also comprises one or more of compounds 1-7.

18. The method of clause 16 and clause 17, wherein the second compound is riluzole, edaravone, dextromethorphan/quinidine, sodium phenylbutyrate (PB), tauroursodeoxycholic acid, sodium phenylbutyrate (PB)/tauroursodeoxycholic acid (i.e. AMX0035), SLS-005 (Trehalose), DNL343, CNM-Au8 nanocrystalline gold or ABBV-CLS-7262.

19. The method of clauses 16-18, wherein the composition comprising pridopidine and the second compound are administered in one unit.

20. The method of clauses 16-18, wherein the composition comprising pridopidine and the second compound are administered in more than one unit.

21. The method of any one of clauses 16-20, wherein the second compound is riluzole.

22. The method of clause 21, wherein 10 mg-200 mg or 50 mg, 100 mg or 200 mg of riluzole is administered to the subject per day.

23. The method of any one of clauses 17-22, wherein the riluzole is administered orally.

24. The method of any one of clauses 17-20, wherein the second compound is edaravone.

25. The method of clause 24, wherein 5-60 mg or 30 mg or 60 mg of edaravone is administered to the subject per day.

26. The method of any one of clauses 17-20 and 24-25, wherein the edaravone is administered by intravenous infusion.

27. The method of any one of clauses 17-20 and 24-26, where the edaravone is administered once per day for 14 days or 10 days followed by a 14-day drug-free period.

28. The method of any one of clauses 17-20, wherein the second compound is dextromethorphan/quinidine.

29. The method of clause 28, wherein 10, 20, or 40 mg of dextromethorphan is administered to the subject per day and 5, 10 or 20 mg of quinidine is administered to the subject per day.

30. The method of any one of clauses 28-29, wherein the dextromethorphan/quinidine is administered orally.

31. The method of any one of clauses 17-30, wherein the amount of pridopidine and the amount of the second compound are administered simultaneously.

32. The method of any one of clauses 17-30, wherein the administration of the second compound substantially precedes the administration of pridopidine.

33. The method of any one of clauses 17-30, wherein the administration of pridopidine substantially precedes the administration of the second compound.

34. The method of any one of clauses 17-30, wherein the subject is receiving edaravone therapy, dextromethorphan/quinidine therapy, riluzole therapy, SLS-005 (Trehalose) therapy, DNL343 therapy, CNM-Au8 nanocrystalline gold therapy or ABBV-CLS-7262 therapy prior to initiating pridopidine therapy.

35. The method of clause 34, wherein the subject is receiving edaravone therapy, dextromethorphan/quinidine therapy, riluzole therapy, SLS-005 (Trehalose) therapy, DNL343 therapy, CNM-Au8 nanocrystalline gold therapy or ABBV-CLS-7262 therapy for at least 24 weeks, 28 weeks, 48 weeks, or 52 weeks prior to initiating pridopidine therapy.

36. The method of any one of clauses 17-30, wherein the subject is receiving pridopidine therapy prior to initiating edaravone therapy, dextromethorphan/quinidine therapy, riluzole therapy SLS-005 (Trehalose) therapy, DNL343 therapy, CNM-Au8 nanocrystalline gold therapy or ABBV-CLS-7262 therapy.

37. The method of clause 36, wherein the subject is receiving pridopidine therapy for at least 24 weeks, 28 weeks, 48 weeks, or 52 weeks prior to initiating edaravone therapy, dextromethorphan/quinidine therapy, riluzole therapy, SLS-005 (Trehalose) therapy, DNL343 therapy, CNM-Au8 nanocrystalline gold therapy or ABBV-CLS-7262 therapy.

38. The method of any one of clauses 17-37, wherein each of the amount of the second compound when taken alone, and the amount of pridopidine when taken alone is effective to treat the subject

39. The method of any one of clauses 17-37 wherein either the amount of the second compound when taken alone, the amount of pridopidine when taken alone, or each such amount when taken alone is not effective to treat the subject.

40. The method of any one of clauses 17-37, wherein either the amount of the second compound when taken alone, the amount of pridopidine when taken alone, or each such amount when taken alone is less effective to treat the subject.

41. The method of any one of clauses 17-40, wherein the pridopidine is administered adjunctively to the second compound.

42. The method of any one of clauses 17-40, wherein the second compound is administered adjunctively to the pridopidine.

43. The method of any one of clauses 1-42, wherein a loading dose of an amount different from the intended dose is administered for a period of time at the start of the periodic administration.

44. A method of enhancing BDNF axonal transport in motor neurons in a subject afflicted with ALS comprising administering to the subject an amount of pridopidine effective to enhance BDNF axonal transport in the subject's motor neurons.

45. A method of improving NMJ formation and function in a subject afflicted with ALS comprising administering to the subject an amount of pridopidine effective to improve NMJ formation and muscle contraction function in the subject.

46. A method of improving innervation rate of muscle tissue in a subject afflicted with ALS comprising administering to the subject an amount of pridopidine effective to improve innervation rate in the subject.

47. A method of enhancing motor neuron axonal growth in a subject afflicted with ALS comprising administering to the subject an amount of pridopidine effective to enhance motor neuron axonal growth in the subject.

48. A method of enhancing muscle contraction in a subject afflicted with ALS comprising administering to the subject an amount of pridopidine effective to enhance the muscle contraction in the subject.

49. A method of restoring muscle contraction in a subject afflicted with ALS comprising administering to the subject an amount of pridopidine effective to improve the muscle contraction in the subject.

50. A pharmaceutical composition comprising an effective amount of pridopidine for use in treating a subject afflicted with ALS.

51. Use of an amount of pridopidine for the manufacture of a medicament for use in treating a subject afflicted with ALS.

52. A package comprising:
a) a pharmaceutical composition comprising an amount of pridopidine; and optionally
b) instructions for use of the pharmaceutical composition to treat a subject afflicted with ALS.

53. A therapeutic package for dispensing to, or for use in dispensing to, a subject, which comprises:
a) one or more-unit doses, each such unit dose comprising an amount of pridopidine thereof, wherein the amount of said pridopidine in said unit dose is effective, upon administration to said subject, to treat ALS in the subject, and
b) a finished pharmaceutical container therefore, said container containing said unit dose or unit doses, said container further containing or comprising labeling directing the use of said package in the treatment of a subject afflicted with ALS.

54. A package comprising:
a) a first pharmaceutical composition comprising an amount of pridopidine and a pharmaceutically acceptable carrier.
b) a second pharmaceutical composition comprising an amount of a second compound which is riluzole, edaravone, dextromethorphan/quinidine, sodium phenylbutyrate (PB), tauroursodeoxycholic acid, sodium phenylbutyrate (PB)/tauroursodeoxycholic acid (i.e. AMX0035), SLS-005 (Trehalose), DNL343, CNM-Au8 nanocrystalline gold or ABBV-CLS-7262 and a pharmaceutically acceptable carrier; and optionally
c) instructions for use of the first and second pharmaceutical compositions together to treat a subject afflicted with ALS.

55. The package of clause 52, wherein the amount of the second compound and the amount of pridopidine are prepared to be administered simultaneously or contemporaneously.

56. A therapeutic package for dispensing to, or for use in dispensing to, a subject afflicted with ALS, which comprises:
a) one or more-unit doses, each such unit dose comprising:
i) an amount of pridopidine and
ii) an amount of a second compound which is riluzole, edaravone, dextromethorphan/quinidine, SLS-005 (Trehalose), DNL343, CNM-Au8 nanocrystalline gold or ABBV-CLS-7262;
wherein the respective amounts of said pridopidine and the second compound in said unit dose are effective, upon concomitant administration to said subject, to treat the subject, and
b) a finished pharmaceutical container therefore, said container containing said unit dose or unit doses, said container further containing or comprising labeling directing the use of said package in the treatment of said subject.

57. A pharmaceutical composition comprising an amount of pridopidine and an amount of a second compound which is riluzole, edaravone, dextromethorphan/quinidine, SLS-005 (Trehalose), DNL343, CNM-Au8 nanocrystalline gold or ABBV-CLS-7262.

58. The pharmaceutical composition of clause 55 for use in treating a subject afflicted with ALS, wherein the pridopidine and the second compound are prepared to be administered simultaneously, contemporaneously, or concomitantly.

59. A pharmaceutical composition in unit dosage form, useful in treating a subject afflicted with ALS, which comprises:
a) an amount of pridopidine.
b) an amount of second compound which is riluzole, edaravone, dextromethorphan/quinidine, SLS-005 (Trehalose), DNL343, CNM-Au8 nanocrystalline gold or ABBV-CLS-7262,
wherein the respective amounts of said second compound and said pridopidine in said composition are effective, upon concomitant administration to said subject of one or more of said unit dosage forms of said composition, to treat the subject.

60. A pharmaceutical composition comprising an amount of pridopidine for use in treating a subject afflicted with ALS as an add-on therapy to a second compound which is riluzole, edaravone, SLS-005 (Trehalose), DNL343, CNM-Au8 nanocrystalline gold, ABBV-CLS-7262 or dextromethorphan/quinidine.

61. A pharmaceutical composition comprising an amount of pridopidine for use in treating a subject afflicted with ALS simultaneously, contemporaneously, or concomitantly with a second compound which is riluzole, edaravone, dextromethorphan/quinidine, SLS-005 (Trehalose), DNL343, CNM-Au8 nanocrystalline gold or ABBV-CLS-7262.

62. A pharmaceutical composition comprising an amount of a compound which is riluzole, edaravone or dextromethorphan/quinidine for use in treating a subject afflicted with ALS as an add-on therapy to pridopidine.

63. A pharmaceutical composition comprising an amount of a compound which is riluzole, edaravone or dextromethorphan/quinidine for use in treating a subject afflicted with ALS simultaneously, contemporaneously, or concomitantly with pridopidine.

64. A compound which is riluzole, edaravone, dextromethorphan/quinidine, SLS-005 (Trehalose), DNL343, CNM-Au8 nanocrystalline gold or ABBV-CLS-7262 for use as an add-on therapy to pridopidine in treating a subject afflicted with ALS.

65. Pridopidine for use as an add-on therapy to a compound which is riluzole, edaravone dextromethorphan/quinidine or SLS-005 (Trehalose), DNL343, CNM-Au8 nanocrystalline gold or ABBV-CLS-7262 in treating a subject afflicted with ALS.

66. The add-on therapy of clause 63, wherein the therapy is for the treatment, prevention, or alleviation of a symptom of ALS.

67. A combination of pridopidine with a compound which is riluzole, edaravone, dextromethorphan/quinidine, SLS-005 (Trehalose), DNL343, CNM-Au8 nanocrystalline gold or ABBV-CLS-7262 for use in the treatment, prevention, or alleviation of a symptom of ALS.

Throughout this application, certain publications and patent application publications are referenced. Full citations for the publications may be found immediately preceding the claims. The disclosures of these publications and patent application publications in their entireties are hereby incorporated by reference into this application in order to describe more fully the state of the art to which this invention relates.

This invention will be better understood by reference to the Experimental Details which follow, but those skilled in the art will readily appreciate that the specific experiments detailed are only illustrative of the invention as described more fully in the claims which follow thereafter.

### EXAMPLES

### Experiment 1

Pridopidine Increases Axonal Transport which is Impaired in SOD1G93A ALS Neurons in a S1R-Mediated Mechanism.

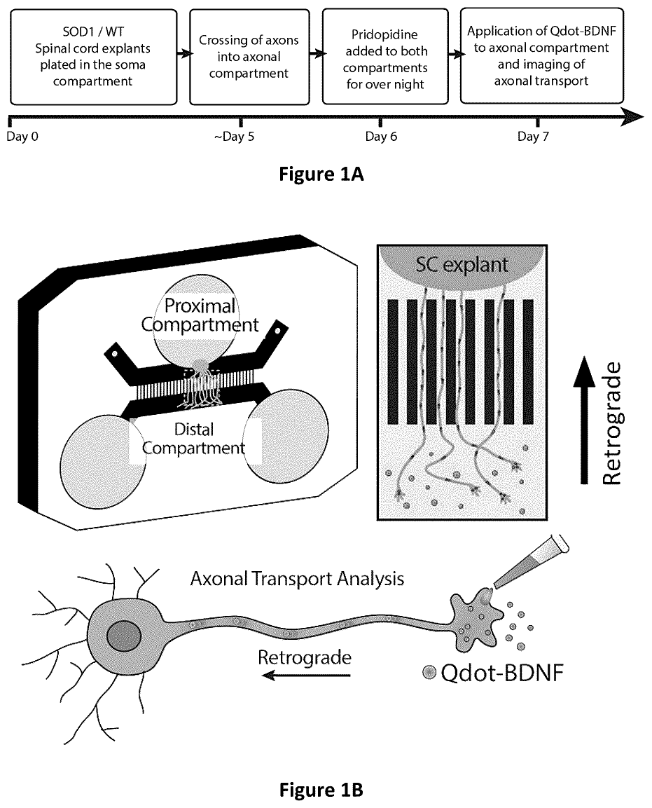

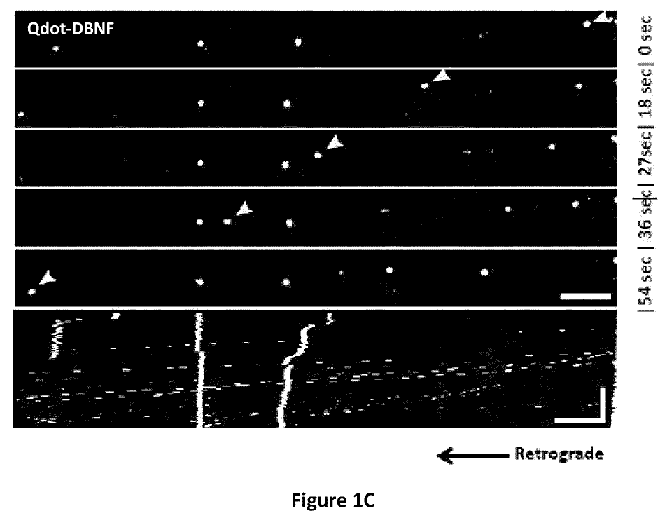

Healthy motor neurons (MN) extend axons over long distances and through varying extracellular microenvironments to form synapses with muscles. The ability of the neuron to maintain this specialized morphology depends on cytoskeletal elements and continuous transport of proteins and organelles to and from the cell body. Cytoskeletal alterations are a major pathway implicated in the pathogenesis of ALS affecting axonal transport, growth, and neuromuscular junction (NMJ) function (Eykens and Robberecht, 2015). Alterations in axonal transport are one of the first cellular processes that occur in neurodegenerative disease, including ALS. Axonal transport was evaluated using an in vitro compartmentalized system of microfluidic chambers (MFC) that separates neuronal cell bodies from their axons and synapses. This enables the study of retrograde/anterograde transport of fluorescently labelled molecules (e.g. Qdot-BDNF) by specific monitoring and manipulation of cellular microenvironments (FIGS. 1A-1C; Zahavi 2015; Ionescu 2016).

Quantum-Dot labeled BDNF (Qdot BDNF) is retrogradely transported in axons of motor neurons grown from spinal cord explants in a microfluidic chamber (MFC). A MFC was used to analyze Qdot BDNF axonal transport. Axonal transport of BDNF in the SOD1 model (SOD1G93A) for ALS has been studied (Bilsland 2010; Perlson 2009; De Vos 2007). The effect of pridopidine on transport of Qdot BDNF along the axons of motor neurons was assessed in spinal cord explants from embryonic day (E) 12.5 SOD1 G93A and wild-type (WT) littermate mice (WT). Experimental workflow for the axonal transport assay (from left to right, FIG. 1A): SOD1G93A or WT spinal cord explants were plated in the proximal compartment of the MFC. At about 5 days post plating, axons began to cross over into the distal compartment. On day 6 post plating, an amount of pridopidine is added to both compartments. On day 7, Qdot-BDNF is added to the distal compartment and axonal transport imaged using a high-resolution spinning-disk confocal microscope. Schematic illustration of microfluidic chamber system (FIG. 1B): Explants planted in the proximal compartment extend axons to the distal compartment, where Qdot-BDNF is applied exclusively prior to visualization.

Spinning disk confocal microscopy was used to track Qdot BDNF along the axons of motor neuron explant cultures. Time lapse images of Qdot-BDNF axonal transport as acquired at 60× magnification (FIG. 1C). Arrowheads point to a single Qdot-BDNF particle that is retrogradely transported (left) towards the cell body. Scale bar: 10 μm. Bottom panel shows a kymograph, which plots distance travelled over time, of a complete Qdot-BDNF time-lapse movie that plots movement along the axon (x axis) as a function of time (y axis). Scale bars: horizontal 10 μm; vertical 100 seconds (FIG. 1C).

Vehicle and pridopidine were added to both compartments at 2 concentrations (0.1 μM, and 1 μM) on experimental day 6, and Qdot BDNF was added to the distal compartment after overnight incubation with pridopidine (FIGS. 1a and 1b). Six independent biological repeats, from 6 different cultures were tested so that from each culture and explant with neurons/glia ˜250 BDNF particles were followed along the axons in the grooves. Velocity refers to the movement of a single BDNF particle. The experiment was repeated with MNs from mice in which sigma 1 receptor was genetically deleted (S1R KO or S1R−/−) (Langa, 2003). Ventral spinal cord sections from S1R−/− mice embryos were cultured and plated in the MFC as described above, and the axonal transport of Qdot-BDNF was analyzed.

SOD1G93 and S1R−/− explants with or without pridopidine were compared to wild-type littermate controls (WT).

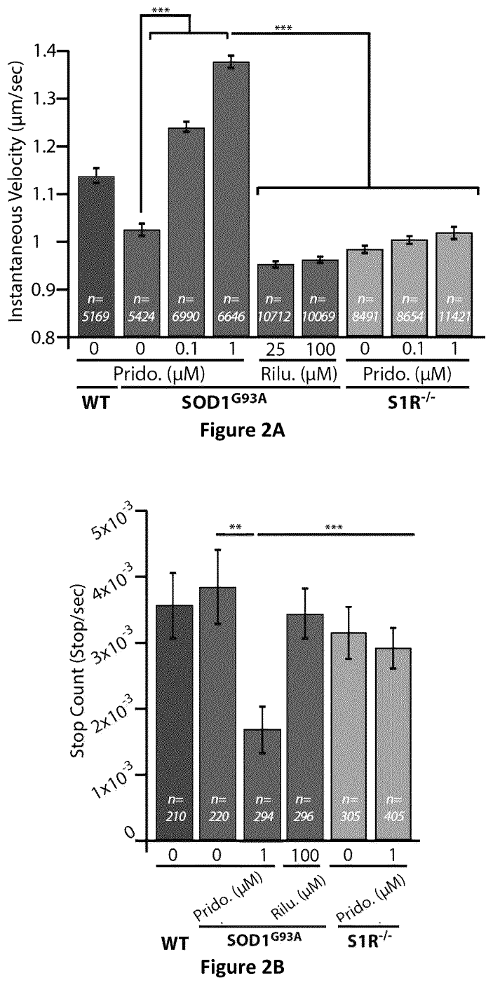

Qdot-BDNF particle tracking was performed on Bitplane Imaris, using the semi-automated spot tracking function. Inclusion criteria for particle analysis: track duration >10 frames; average velocity ≥0.2 μm/sec; stop duration: speed <0.1 μm/sec for 3 frames. Data were then exported to MATLAB for further analysis of particle transport including Instantaneous Velocities (FIG. 2A) from 6 independent cultures; and Stop count (FIG. 2B).

### Results

FIG. 2A demonstrates that pridopidine enhanced BDNF axonal transport instantaneous velocity in SOD1G93A motor neurons. Instantaneous velocity of BDNF retrograde transport is typically reduced in SOD1G93A motor neurons. SOD1G93A MNs showed slower velocities vs the WT MNs. Pridopidine treatment accelerated the instantaneous velocity in SOD1G93A MNs (0.1 μM and 1 μM). Application of 25 μM or 100 μM Riluzole, the standard of care for ALS subjects, to SOD1G93A MNs did not affect the instantaneous velocities. S1R−/− MNs demonstrate reduced velocity of BDNF axonal transport. Pridopidine at either 0.1 μM or 1 μM was not able to recover these defects in S1R KO MNs indicating the effect of pridopidine was exclusively mediated by the S1R (FIG. 2A).

Particle stop count (number of counted stops of Qdot-BDNF per second) was increased in SOD1G93A MNs compared to WT MNs. Pridopidine (1 μM) reduced the number of pauses during axonal transport in SOD1G93A MNs significantly (0.1 μM). Riluzole (100 μM), the standard of care does not demonstrate any significant effect on particle stop count. Pridopidine was unable to rescue particle stop count of Qdot-BDNF in S1R−/− MNs, indicating that pridopidine's effect was mediated by the S1R (FIG. 2B). Data are shown as mean±SEM. **p-value <0.01, ***p-value <0.001, (Student's t-test).

These results demonstrate that pridopidine enhances BDNF axonal transport in SOD1G93A motor neurons and corrects ALS related deficits.

### Experiment 2

Pridopidine Increases Axonal Growth which is Impaired in SOD1G93A Neurons.

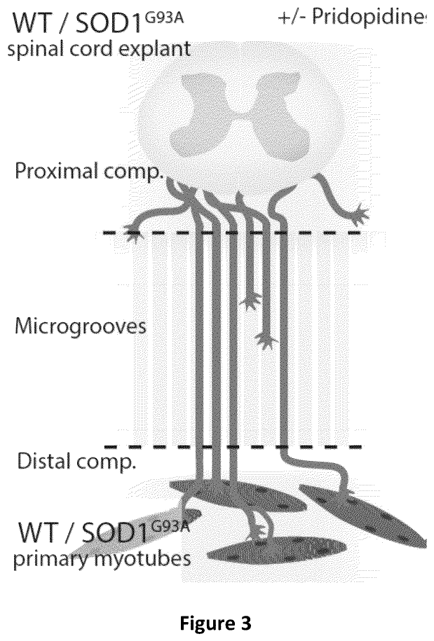

An early event in the pathogenesis of ALS is axonal degeneration. The compartmental co-culture microfluidic chamber system was used to determine whether pridopidine alters axonal degeneration (FIG. 3). Primary muscle cells from presymptomatic (P60) SOD1G93A or WT mice were cultured. On day 6, primary skeletal myoblasts were cultured in the distal compartment of a MFC. About six days later (day 12), ventral spinal cord explants from WT or SOD1G93A E12.5 mouse embryos that express HB9-GFP (a specific motor neuron marker fused to the green fluorescent protein GFP) were plated in the proximal compartment, followed by application of pridopidine or vehicle to both compartments. Pridopidine was refreshed every other day. Two days post explant plating (day 14), motor axon growth and degeneration were evaluated using live imaging on a spinning disc confocal system. Axonal growth was tracked by imaging every 10 min for 8 hrs. Experiments were repeated three times.

### Results

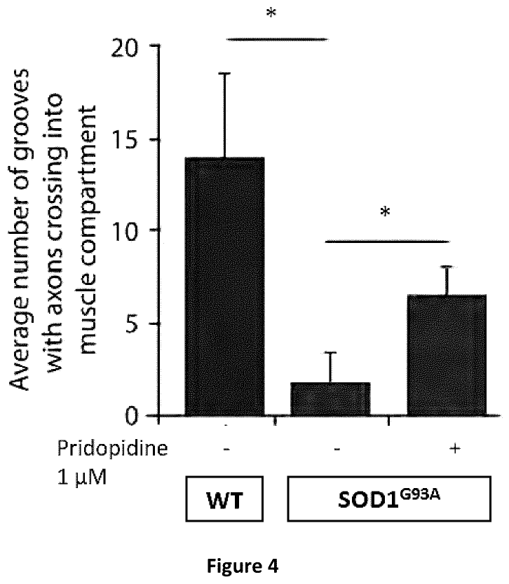

The data demonstrate that pridopidine increased axonal growth (FIG. 4). Myocytes carrying the SOD1G93A mutation have a reduced number of healthy axons that are able to cross into the distal compartment (compartment with muscle cells) of the microfluidic compartmental chamber as compared with WT myocytes (p<0.05). Treatment with 1 μM pridopidine (furthest right bar) significantly increased the number of SOD1G93A axons crossing into the distal compartment (p<0.05). (Y axis is average number of grooves with axons crossing into muscle compartment).

These results demonstrate that pridopidine enhances axonal growth in ALS neurons.

### Experiment 3

### Assessment of the Effect of Pridopidine on Neuromuscular Junction (NMJ) Formation and Function

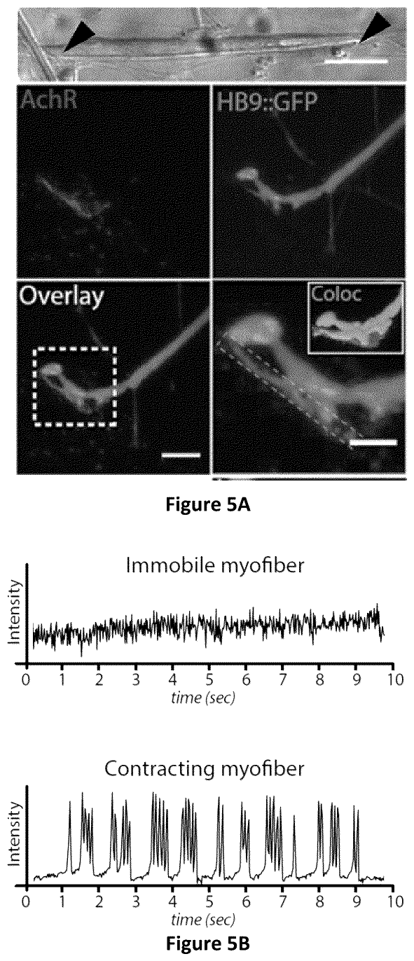

Synapses are earliest cellular compartment disrupted in ALS. To test the ability of pridopidine to affect synapse function in an ALS model, cultures from Experiment 2 described above were grown for approximately four additional days (day 18), when axons extend into the distal compartment and form NMJs. In this co-culture, MN axons formed NMJs on fully differentiated primary myocytes. These can be observed by the co-localization of the post-synaptic marker located in the muscle (AchR, acetyl choline receptor) with the Hb9:GFP neuronal marker. FIG. 5a: Upper panel: Phase-contrast microscope image of a myocyte in the distal compartment connected by axons (arrowheads). Scale bar: 20 μm. Lower panel: High magnification images of myocyte: MN contact points reveal the formation of NMJs as seen by co-localization of post synaptic AChR with HB9::GFP (overlay) axons and 3-dimensional co-localization of pre and post-synaptic markers (coloc). To evaluate NMJ function, movies of muscle contraction were acquired at a frame rate of 30 frames per second for 1000 frames (FIG. 5B). Muscle contraction traces as extracted from intensity over time measurements of muscle contraction show the flat trace of a non-contracting, immobile myocyte (upper), and the trace of a contracting myocyte demonstrating multiple bursting events (lower).

To study the effect of pridopidine on MN and NMJ formation and function, either 0.1 or 1 μM pridopidine or vehicle were added. Measurement of % innervation and innervation-induced contraction in myotubes was evaluated using live cell imaging as previously reported (Ionescu 2016; Zahavi 2015). Briefly, contractile activity of muscles in the distal compartment of the MFC, which were overlapped by at least one axon was examined. Muscles were categorized into two groups: ‘Contracting’ or ‘Non-contracting’, depending on their motile activity during the movie. The motility of muscles was validated by generating intensity-over-time plots for each muscle (FIG. 5B). The number of contracting muscle fibers per chamber was divided by the total number of muscle fibers analyzed in the same chamber, yielding the percentage of contracting myotubes as an output for NMJ activity.

### Results

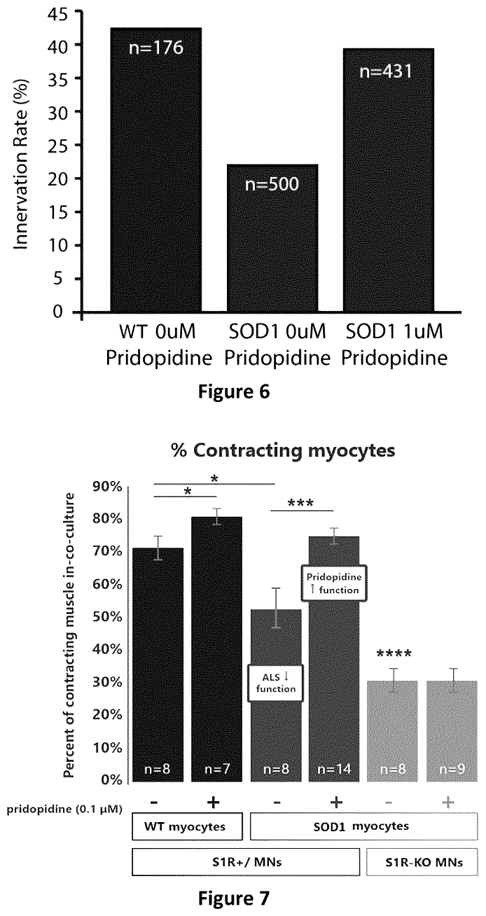

Pridopidine enhanced muscle innervation and increased NMJ function as measured by an increase in the % of contracting myocytes. Innervation rate of muscles carrying the SOD1 mutation was lower compared to WT (wild type) muscles (20% innervation compared to ˜40% in WTs). Pridopidine at 1 μM increased the innervation rate of muscles carrying SOD1 mutation to near WT levels (FIG. 6).

The percent of contracting myotubes was decreased in SOD1 myocytes innervated with WT MNs compared to WT myocytes innervated with WT MNs (50% vs. 70%, p<0.05). Pridopidine (0.1 μM) treatment of SOD1G93A myocytes co-cultured with WT MNs significantly increased the percentage of contracting myocytes to ˜75% (p<0.001) and restored neuromuscular activity to WT levels. SOD1 myocytes demonstrate reduced contractility when innervated with S1R−/− MNs (30% vs. 50% in SOD1 myocytes innervated with WT neurons, p<0.0001). Application of 0.1 μM pridopidine to S1R−/− co-cultures did not restore the neuromuscular activity, as seen for the same concentration of pridopidine in co-cultures with WT neurons. This indicates that the effect of pridopidine is mediated via the S1R. Data are shown as mean±SEM. *p-value <0.05; **p-value <0.01, ***p-value <0.001, ****p-value <0.0001. (Student's t-test).

### Experiment 4

### Pridopidine Activates the ERK Survival Signaling Pathway in WT and SOD1G93A MNs

The extracellular-signal-regulated kinase (ERK) pathway promotes numerous cellular functions including proliferation and differentiation. ERK phosphorylation (activation) in neurons is associated with neurotrophic signaling, such as BDNF, which promotes neuroprotection and neuronal survival (Bonni 1999). It was previously established that pridopidine enhances BDNF signaling in rat striatum through S1R, which in turn, enhances ERK activation (Geva 2016). Primary MN cultures at 2 DIV were starved overnight in neurotrophin- and serum-free medium. The following day, cultures were treated for 30 minutes with pridopidine or with BDNF as a positive control, and the levels of ERK and phosphorylated ERK proteins were measured by Western blot.

### Results

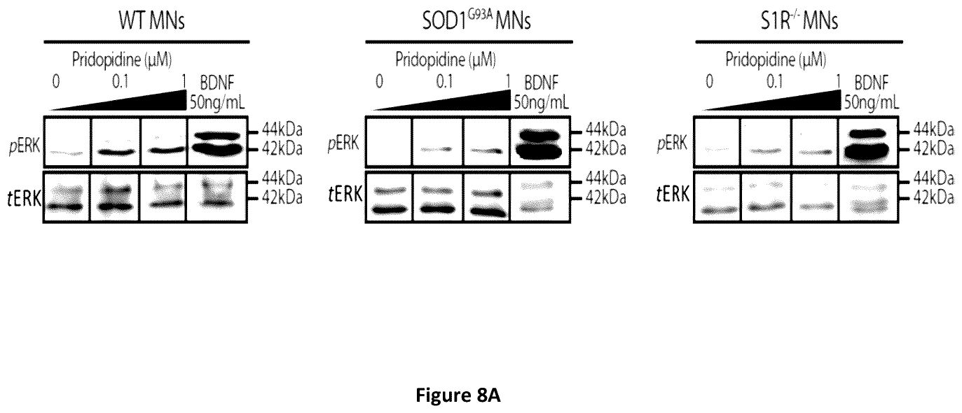

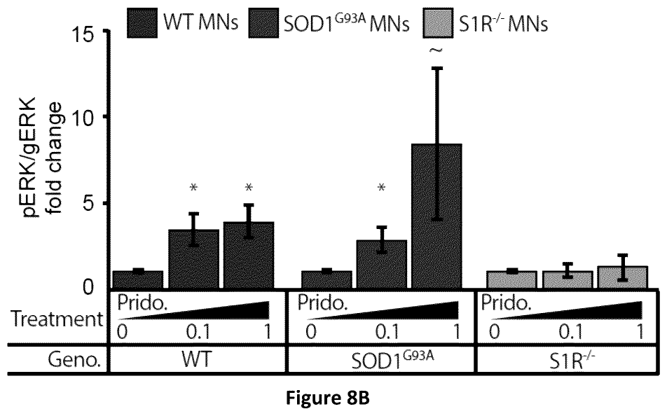

Pridopidine induced a significant increase in phosphorylated ERK (PERK) (0.1 μM and 1 μM), as early as 30 minutes after application in WT (left panel) and SOD1G93A (middle panel) MN cultures. Pridopidine had no effect in S1R−/− MN cultures (right panel) (FIG. 8A), indicating pridopidine's activation of ERK is mediated by the S1R. Quantification of pERK reveals ˜3.5 and ˜4-fold increase in WT MNs following 0.1 μM and 1 μM pridopidine, respectively. SOD1G93A exhibits ˜2.9 and ˜8.5-fold increase in pERK following 0.1 μM and 1 μM pridopidine, respectively. Data are shown as the mean pERK/ERK ratios±SEM. *p-value <0.05, ˜p-value <0.1 (Student's t-test) (FIG. 8B).

### Experiment 5

### Pridopidine Reduces Mutant SOD1 Aggregation in the Spinal Cord of SOD1G93A Mice.

Pridopidine induces neuroprotective properties by activation of the S1R, as demonstrated for its effect on axonal transport, axonal degeneration, NMJ function and ERK activation. The S1R resides on the ER membrane in close proximity to the mitochondrial outer membrane, where the mutant SOD1 protein tends to aggregate in the spinal cord of SOD1G93A mice (Millecamps and Julien 2013). Pre-symptomatic SOD1G93A mice (5 weeks of age) and WT controls were treated with either saline or 30 mg/kg pridopidine, by daily s.c. (subcutaneous) administration for 11 weeks (until 16 weeks of age). At the end of the experiment, lumbar spinal cords (L1-L6) were extracted, fixed, and embedded for cryosectioning. Next, 10 μM sections were prepared and stained with NSC500 dye to visualize SOD1 aggregates (Hammarström 2010). The in vivo effect of pridopidine treatment on the number of mutant SOD1 aggregates in grey and white matter of spinal cord was evaluated.

### Results

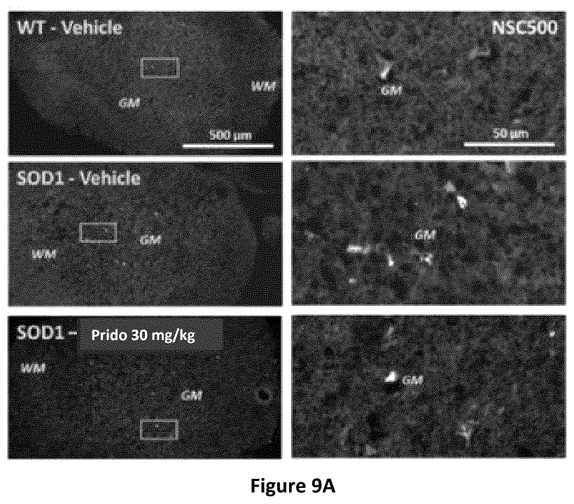

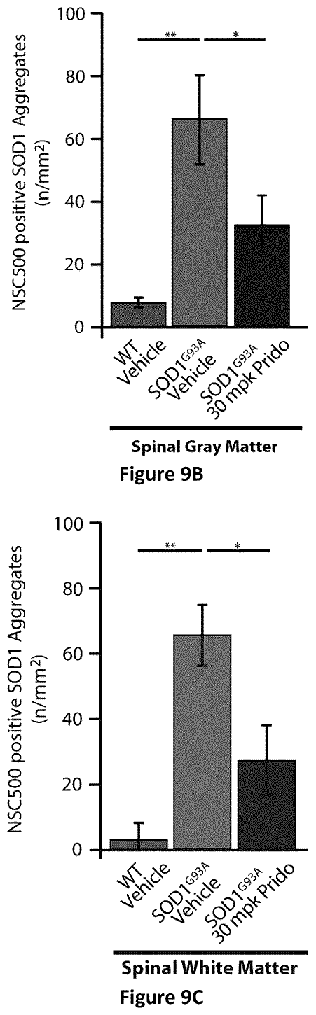

FIG. 9A—Left panel: low magnification representative images of fluorescently labeled spinal cords for 3 mouse groups (WT, SOD1 treated with vehicle control and SOD1 treated with 30 mg/kg pridopidine). Right panel: high magnification images for the regions marked in the left panel by a square. Scale bars: Left panel: 500 μm; Right panel 50 μm. Top to bottom: WT vehicle, SOD1G93A vehicle, SOD1G93A 30 mg/kg, all stained with NSC500 dye to label mutant SOD1 protein aggregates. A significant increase in the number of mSOD1 aggregates was observed in both the gray and white matter of the spinal cords of SOD1G93A mice compared with WT mice. Pridopidine 30 mg/kg significantly reduced the number of aggregates in both the gray (FIG. 9B) and white (FIG. 9C) matters of SOD1G93A spinal cords by ˜50% (FIGS. 9A-9C). Data are shown as the mean±SEM. *p-value <0.05; **p-value <0.01 (one-way ANOVA followed by Fisher's LSD post hoc tests). (FIGS. 9B-9C y-axis is number of NSC500-positive SOD1 aggregates per squared mm).

### Experiment 6

### Pridopidine Reduces Muscle Fiber Atrophy and Increases NMJ Preservation in SOD1 Mice

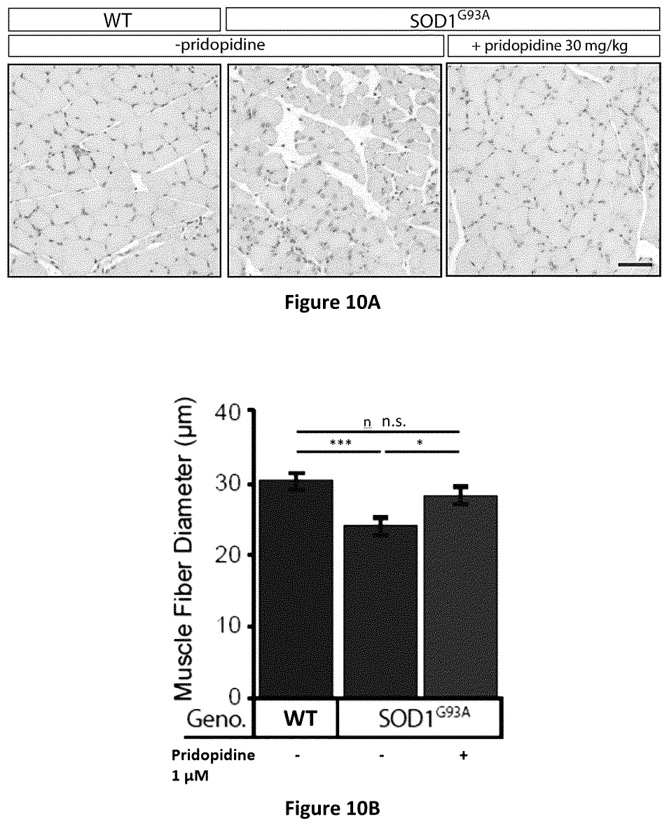

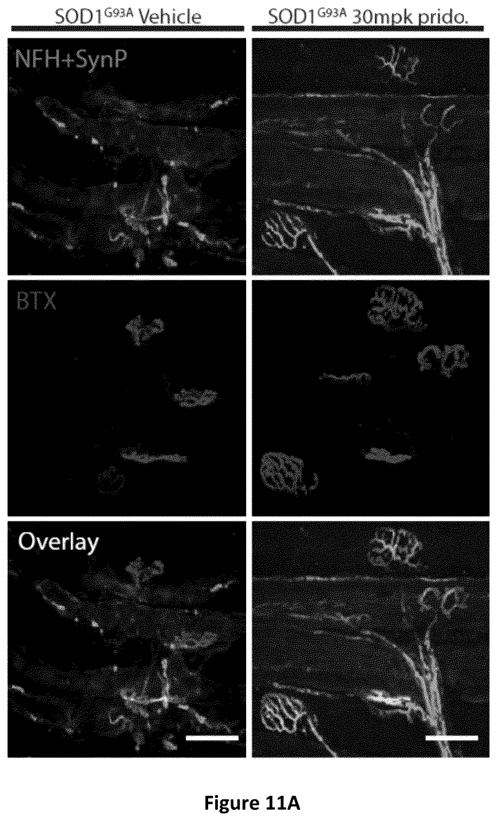

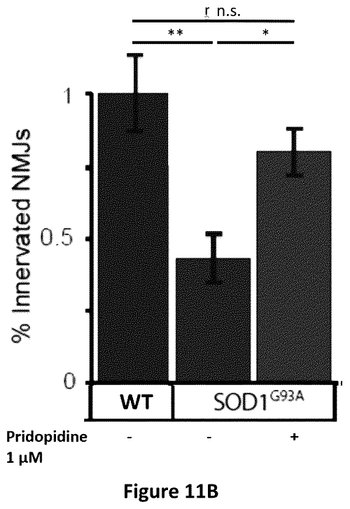

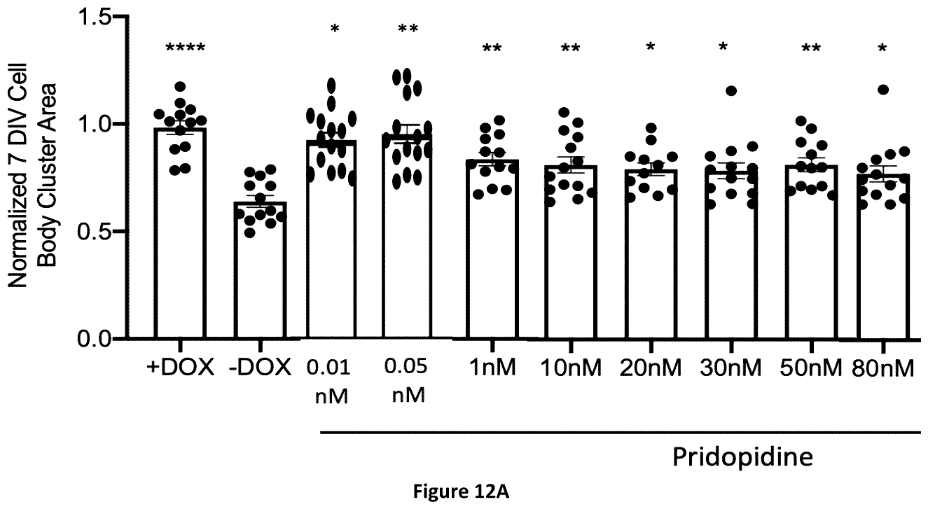

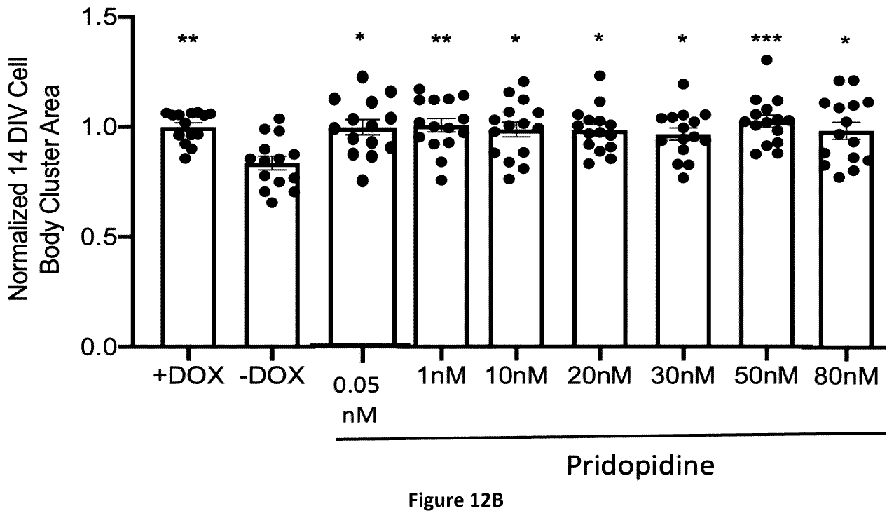

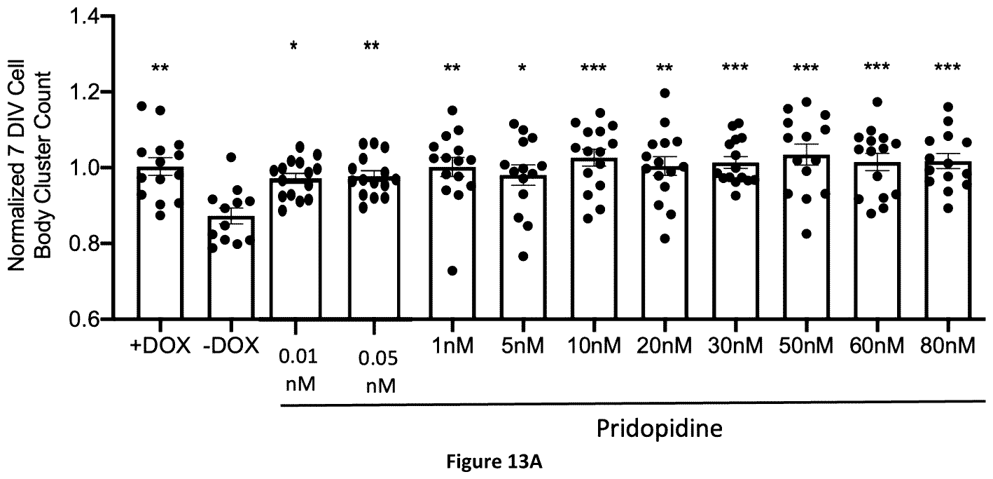

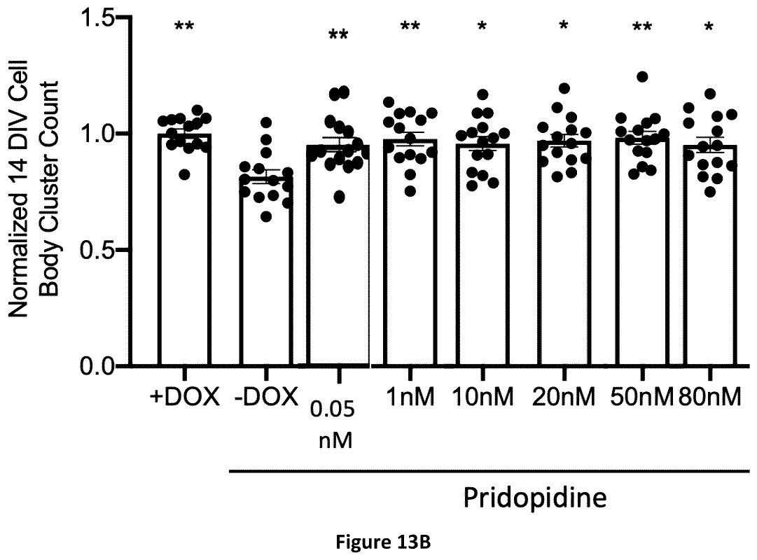

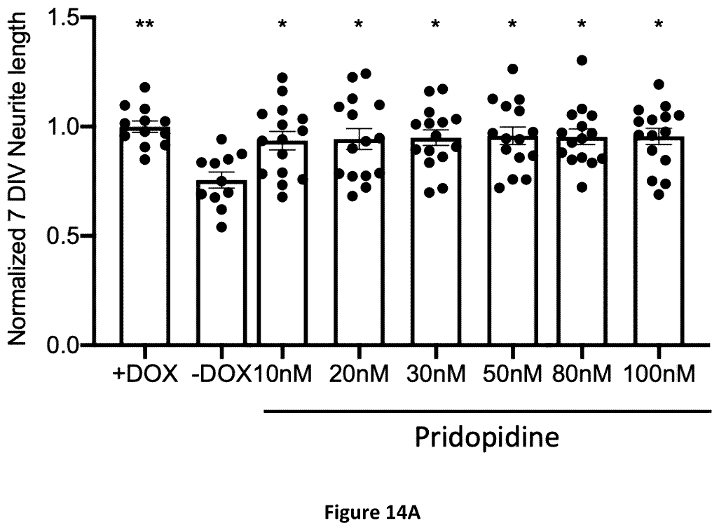

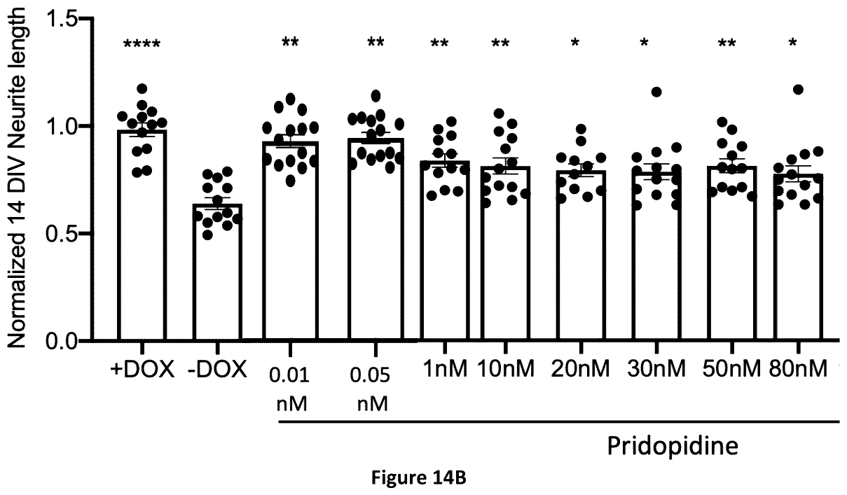

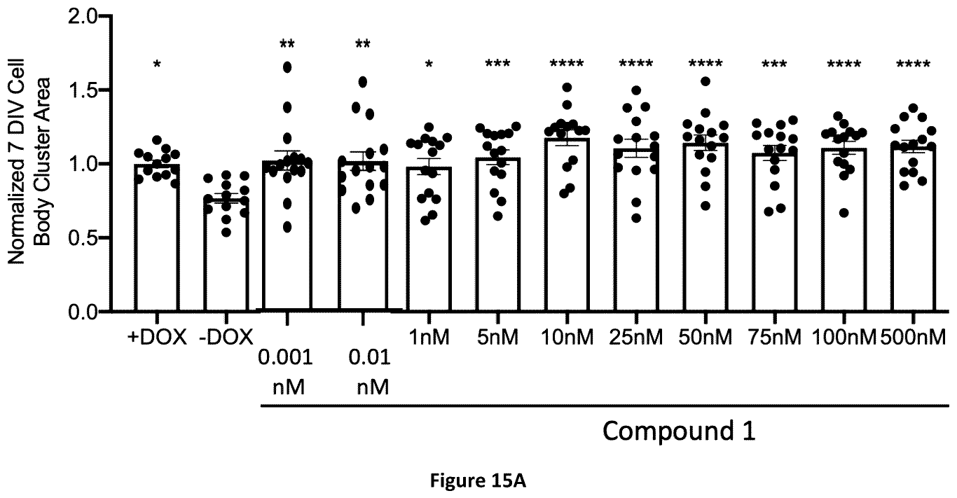

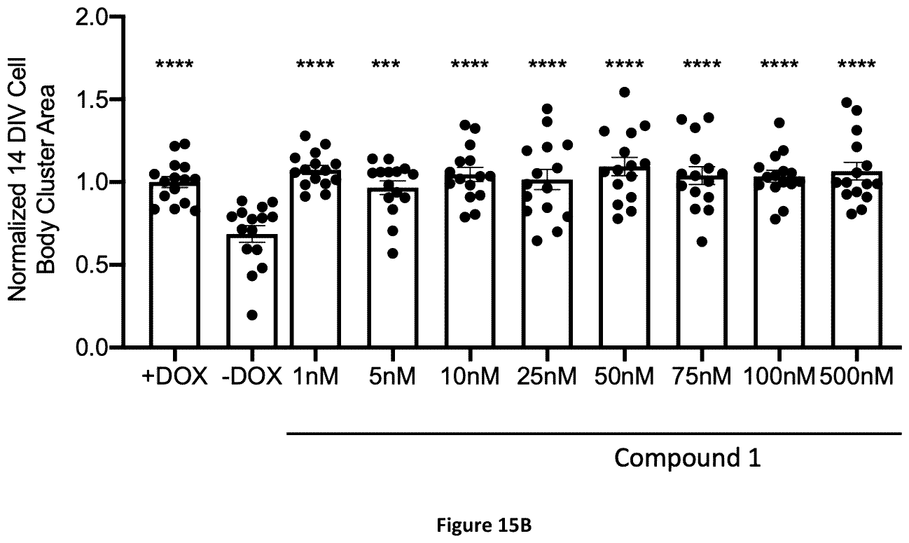

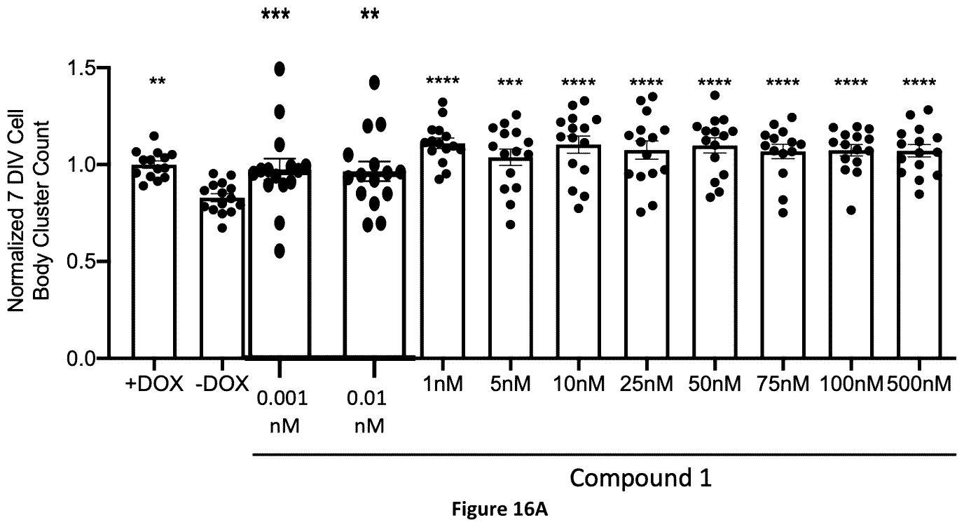

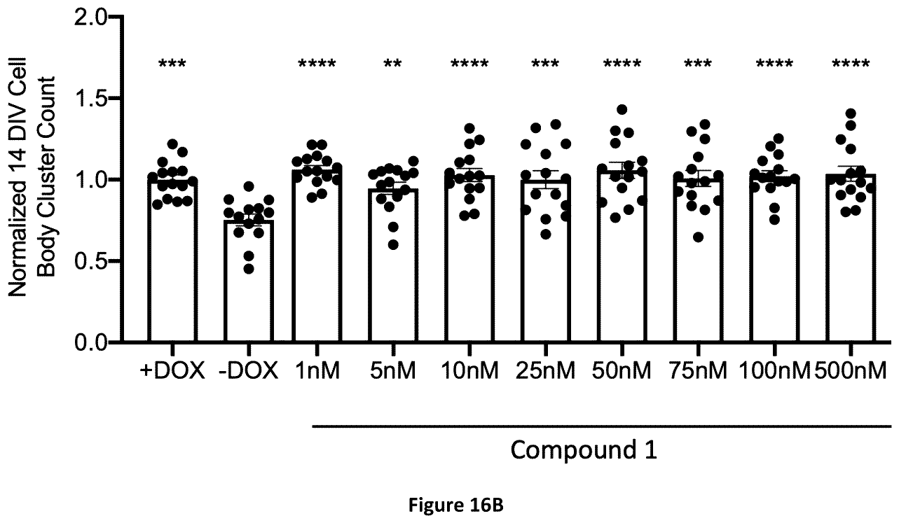

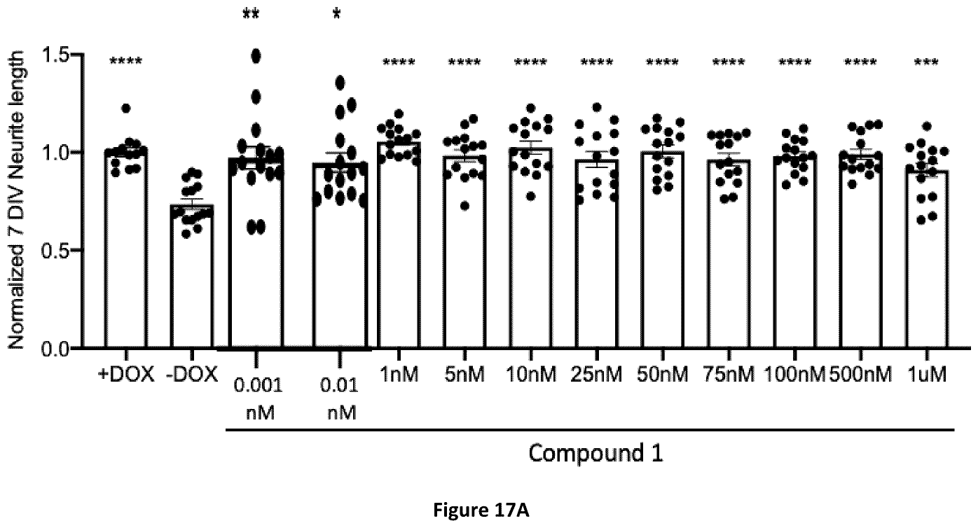

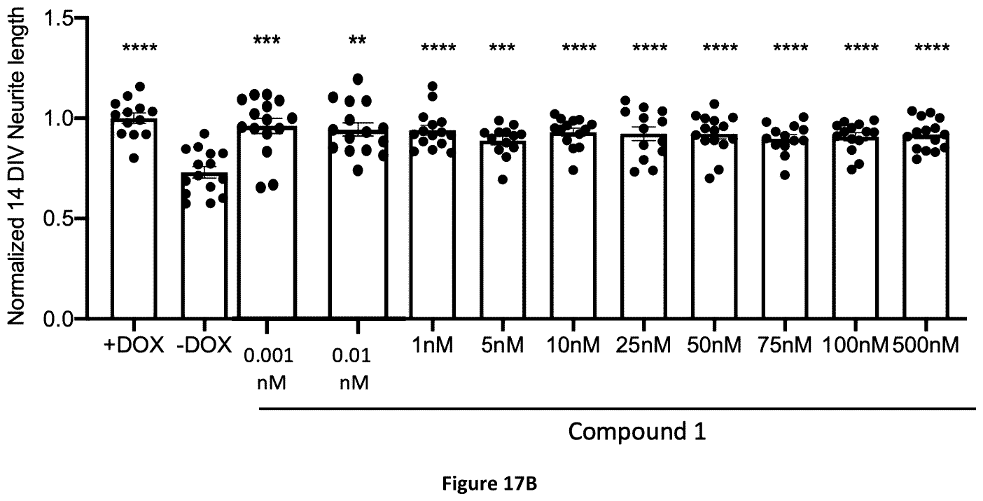

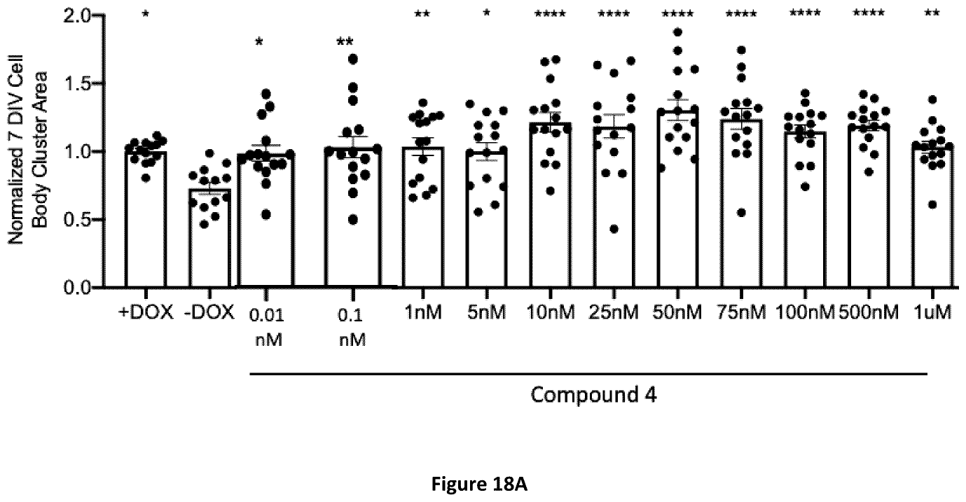

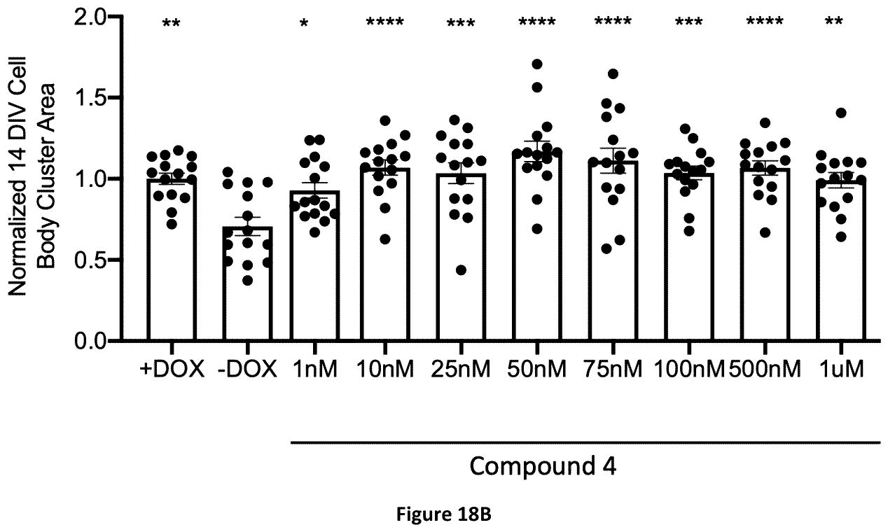

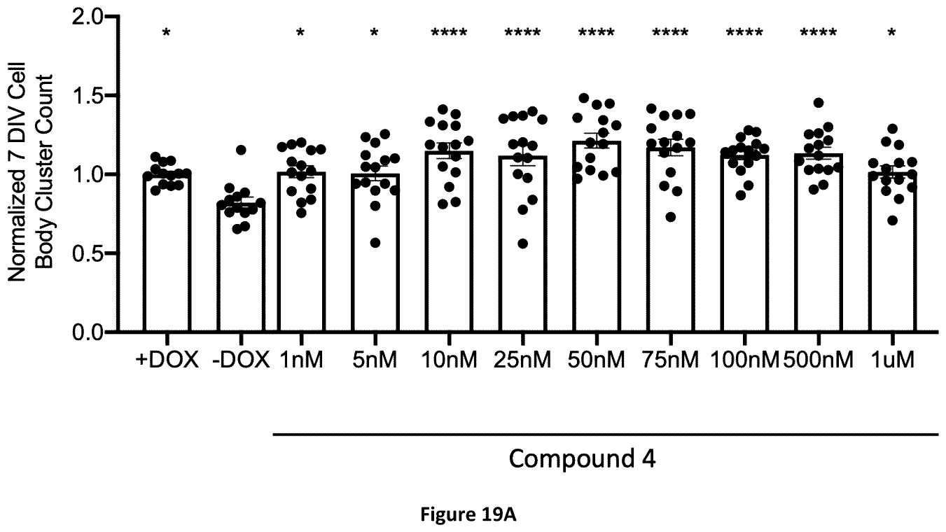

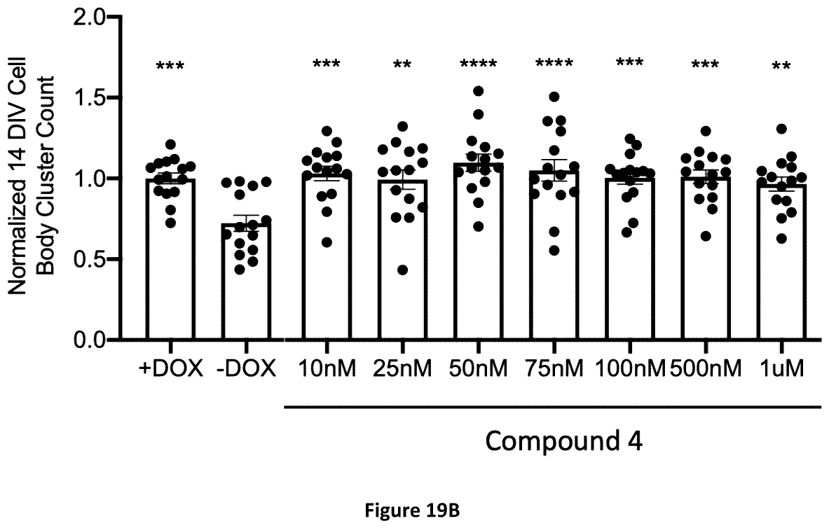

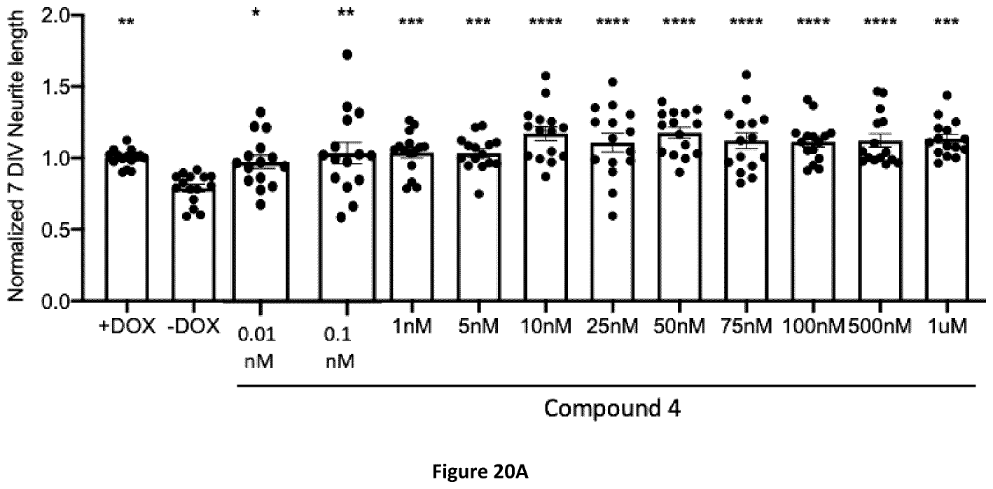

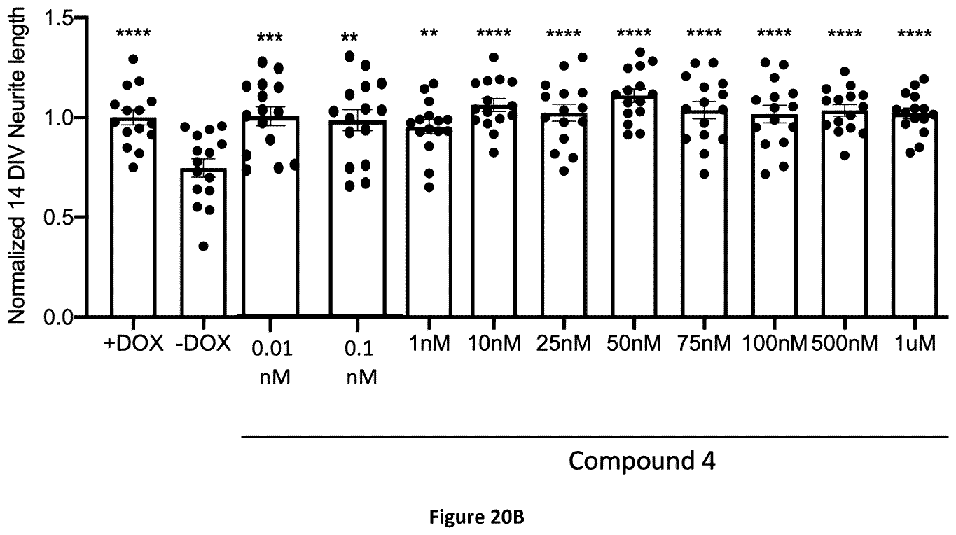

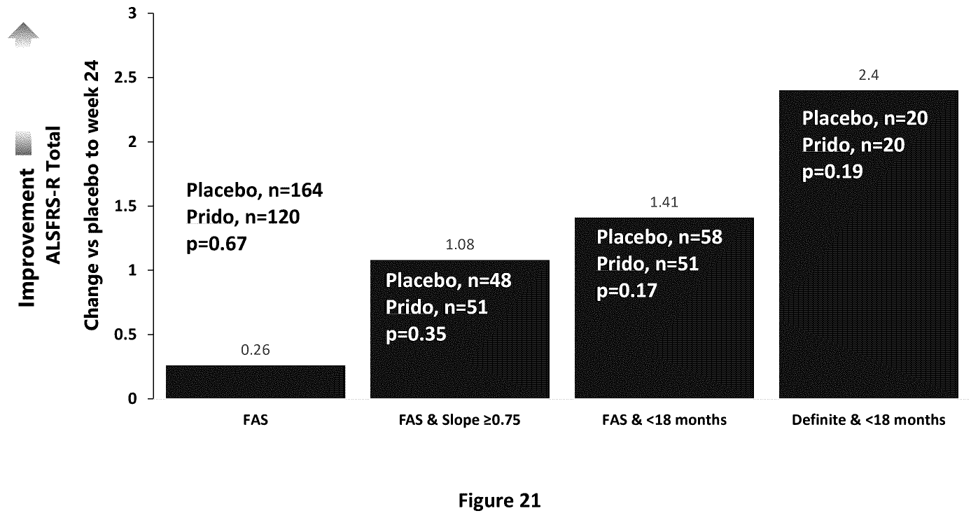

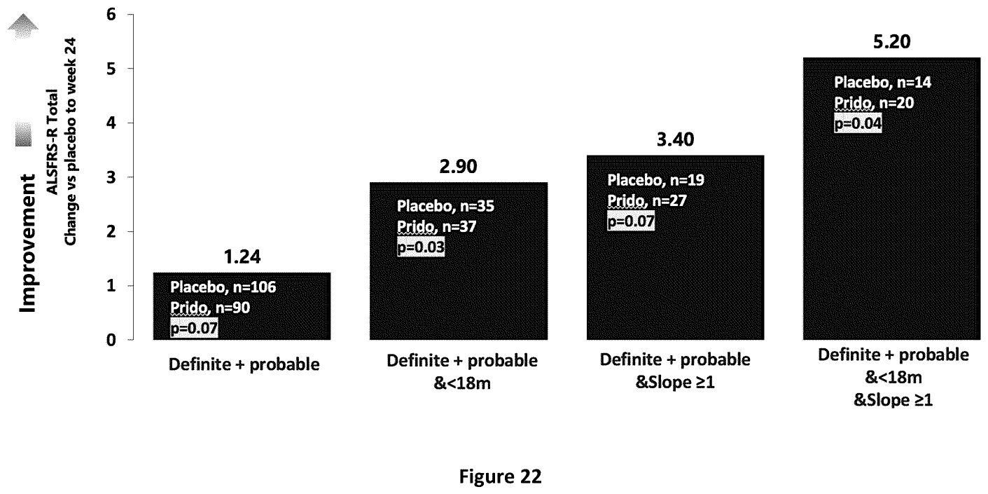

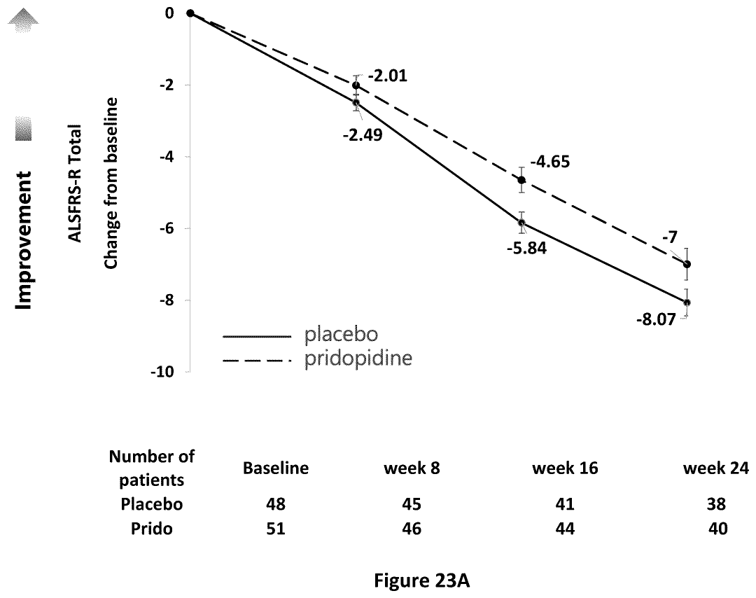

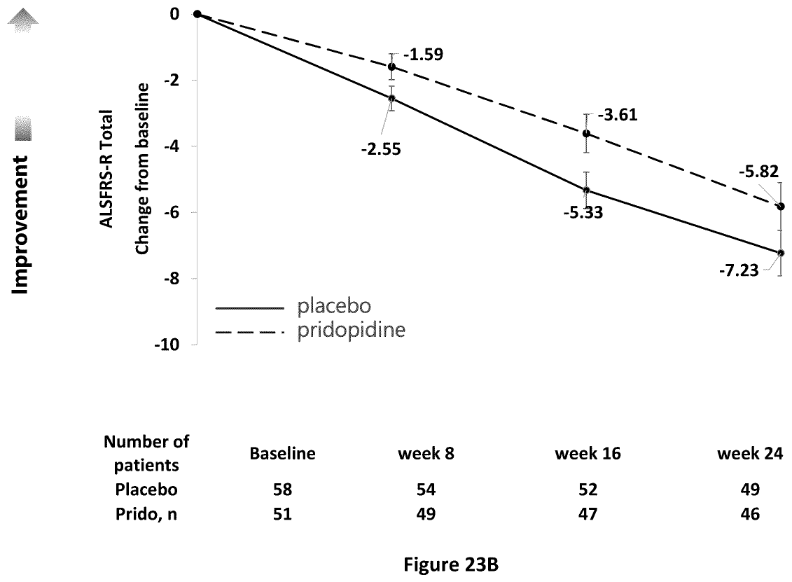

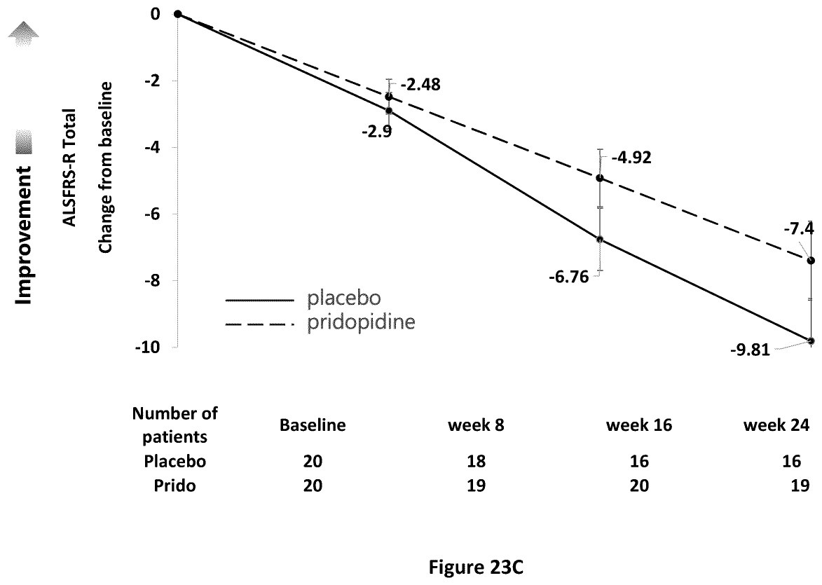

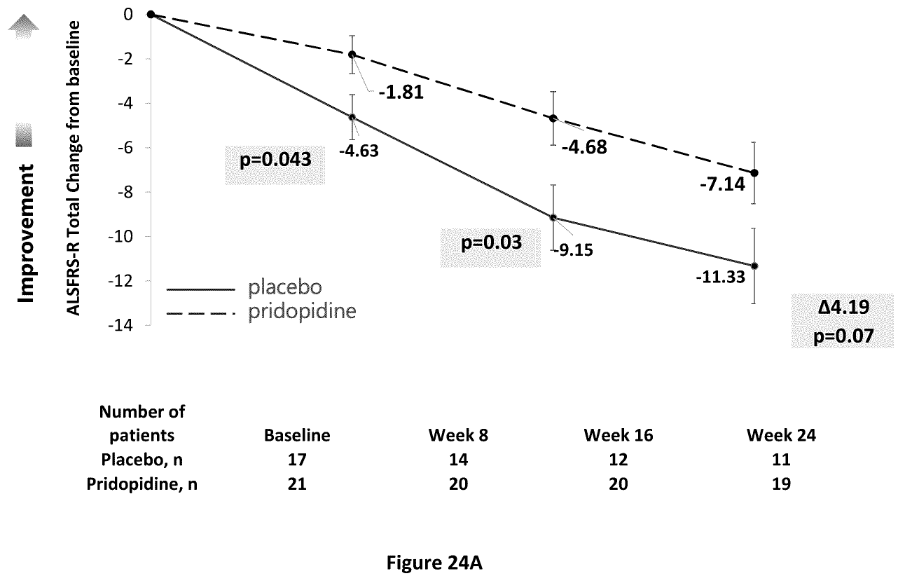

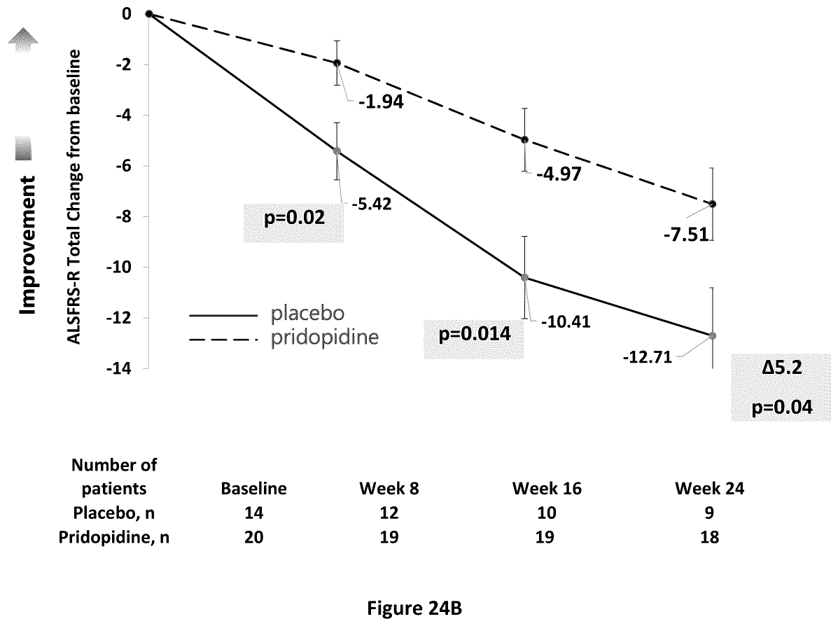

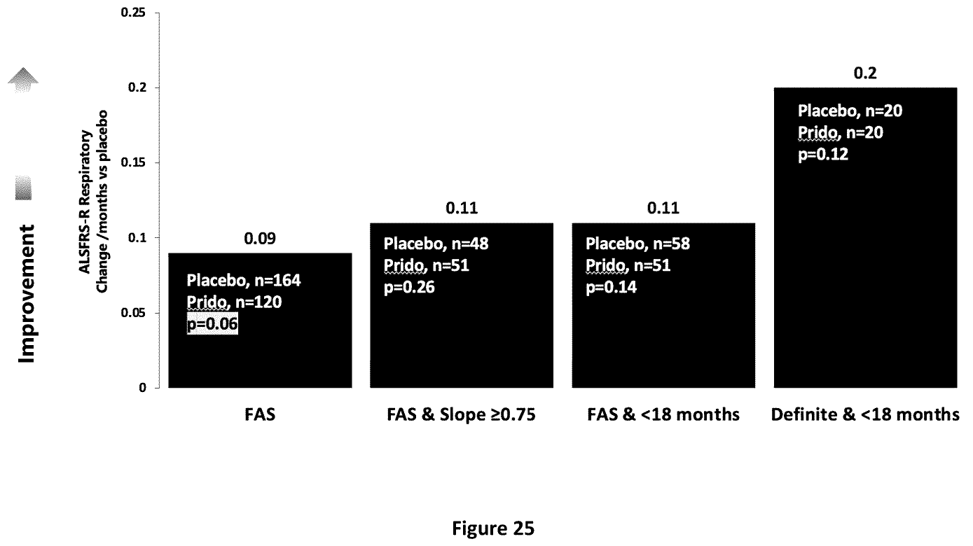

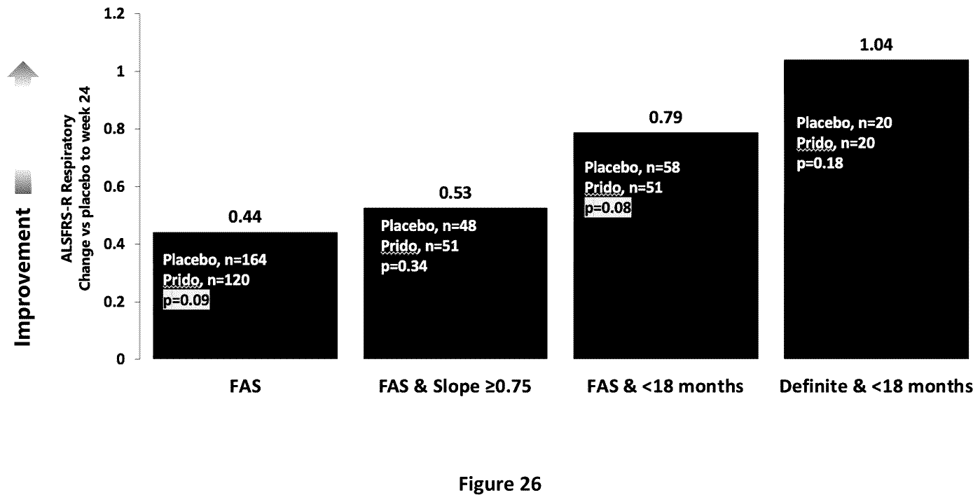

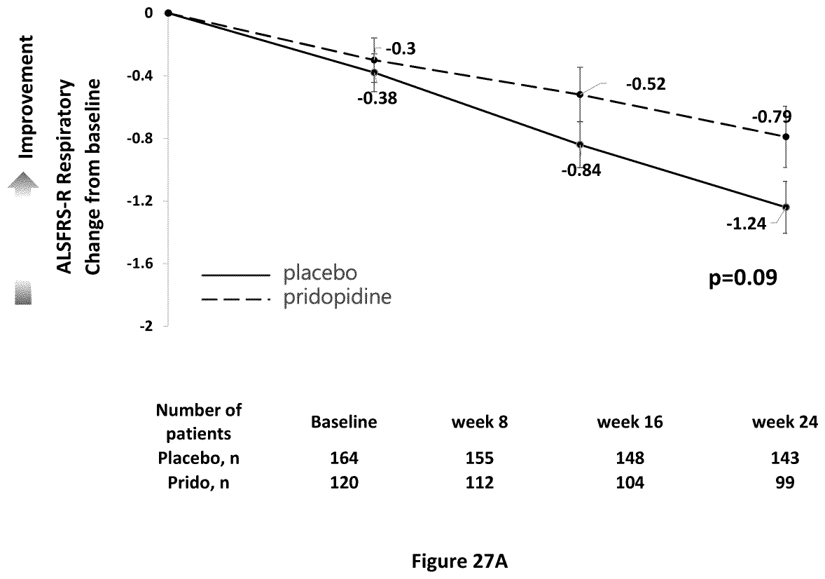

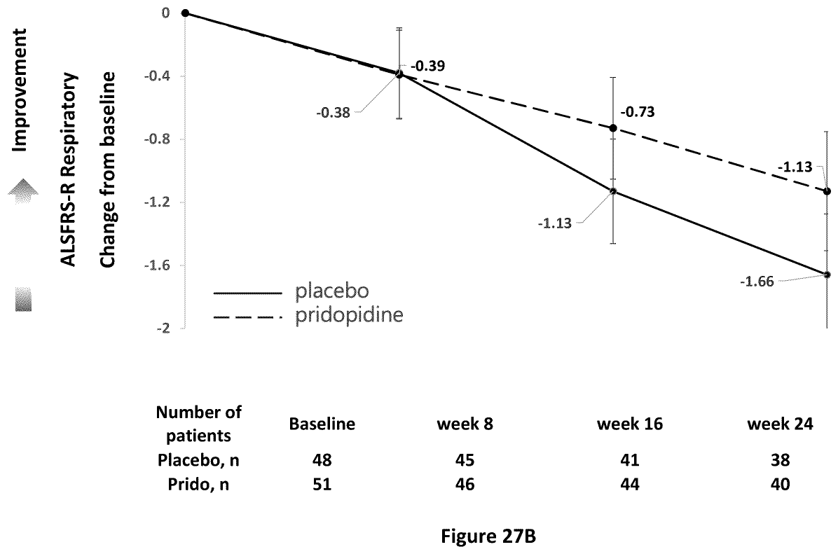

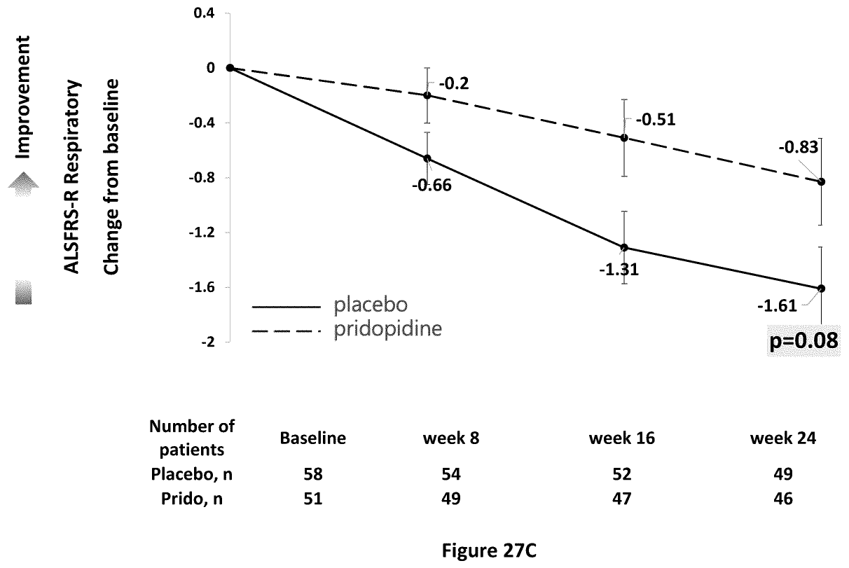

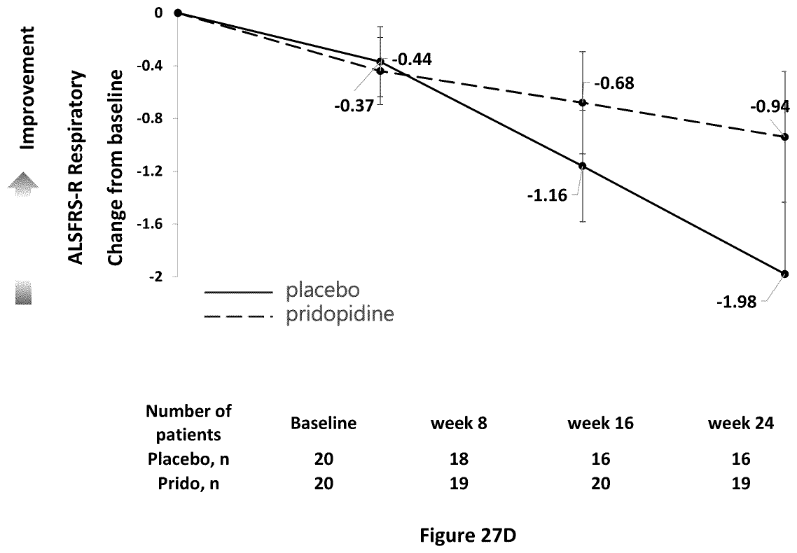

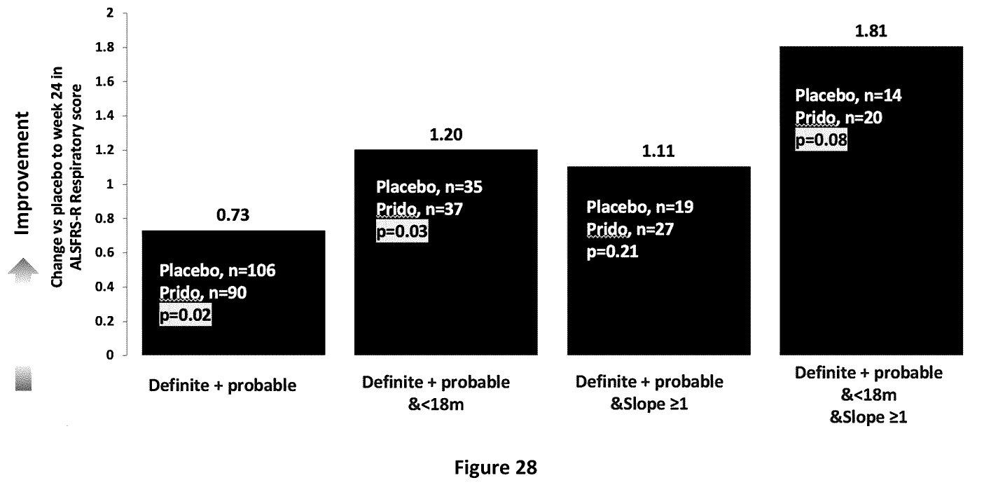

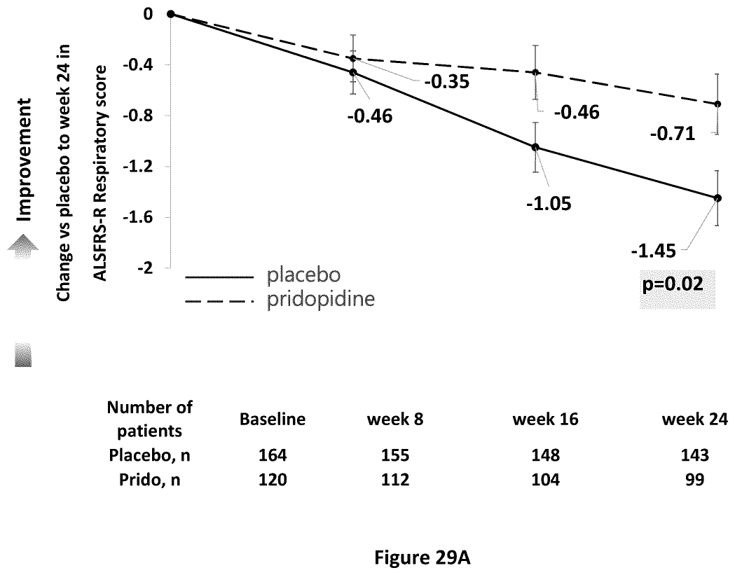

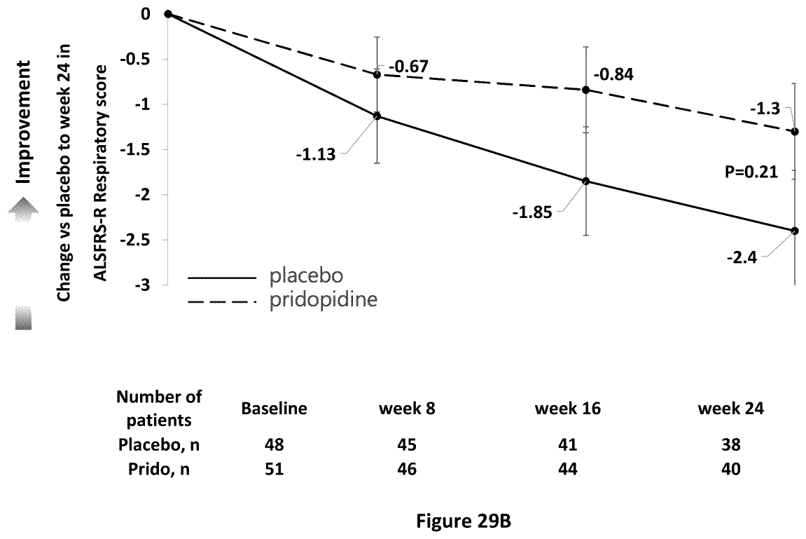

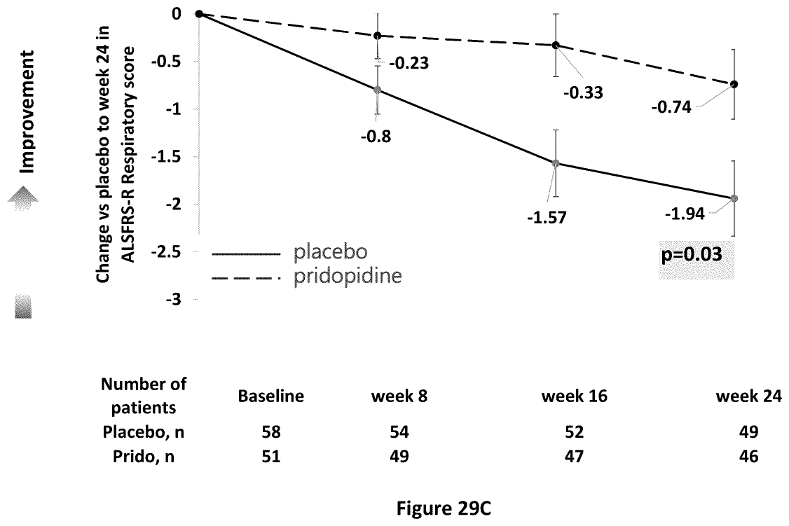

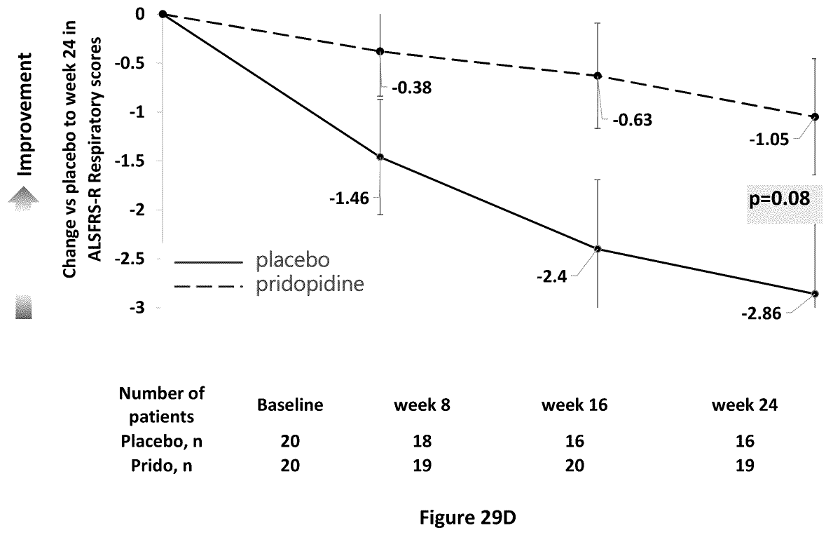

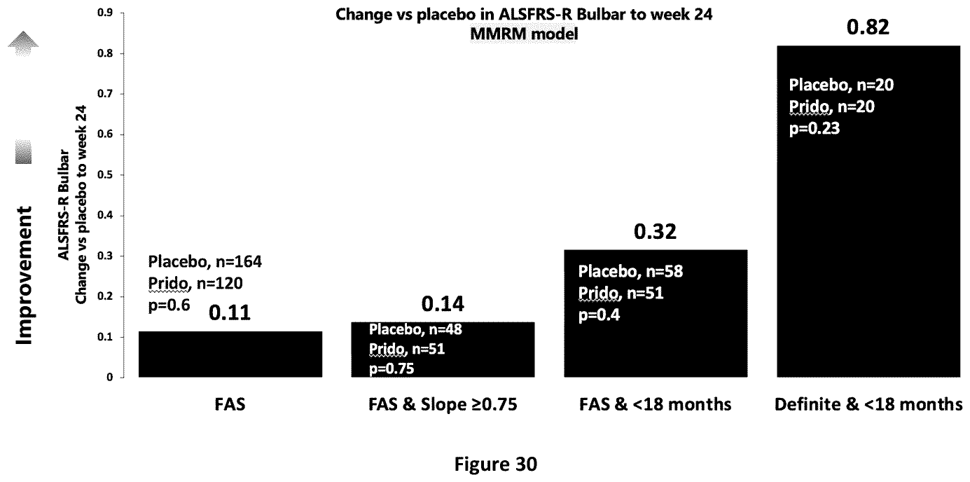

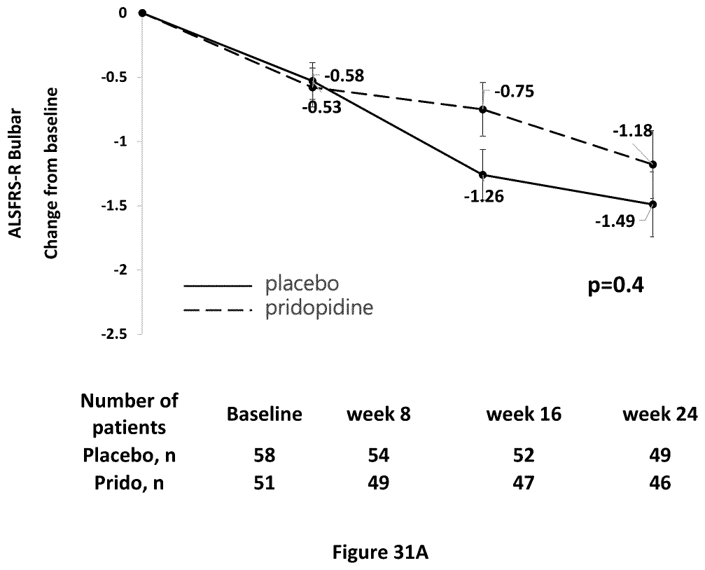

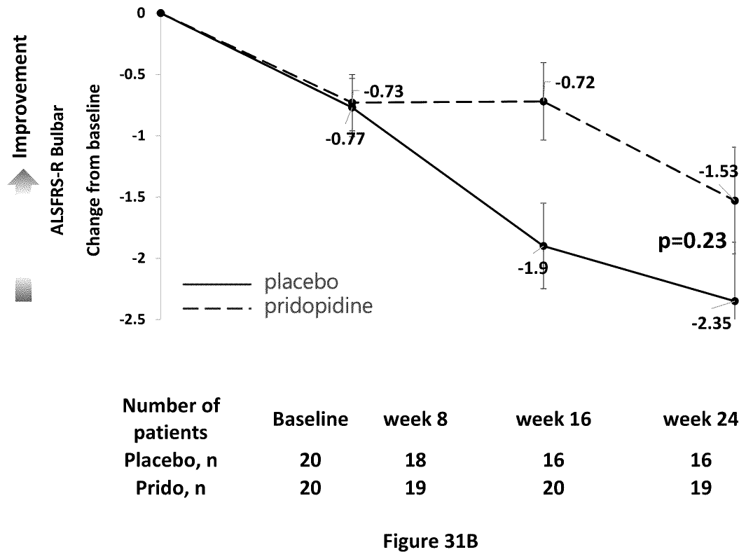

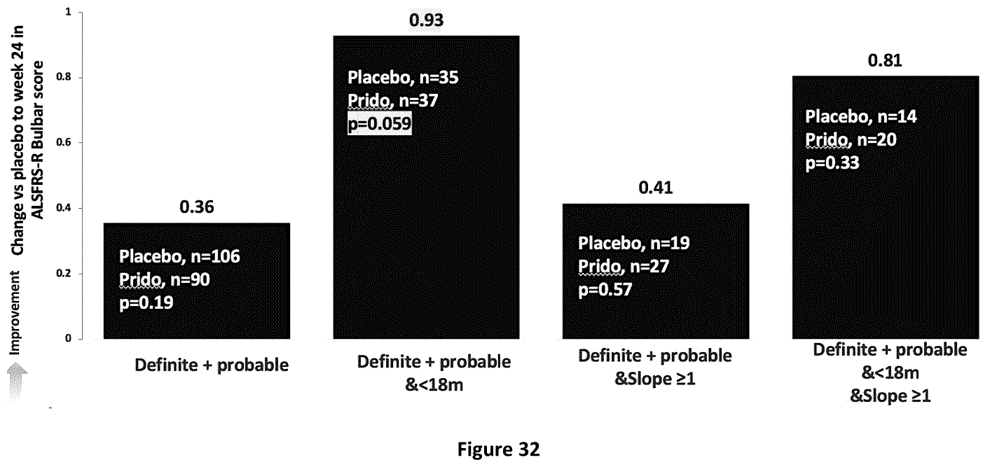

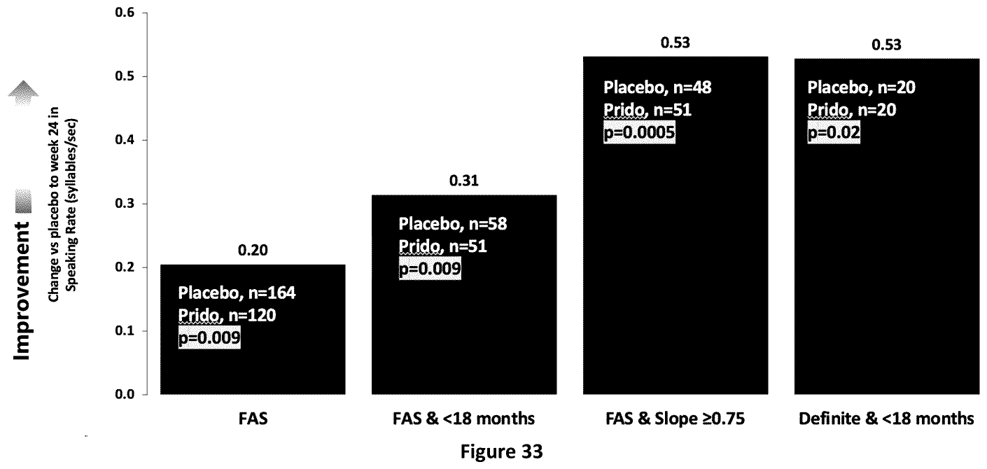

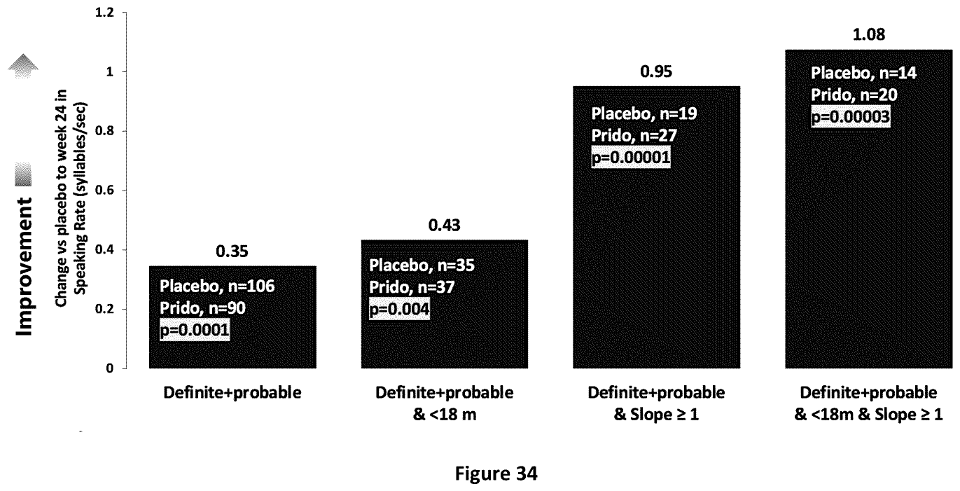

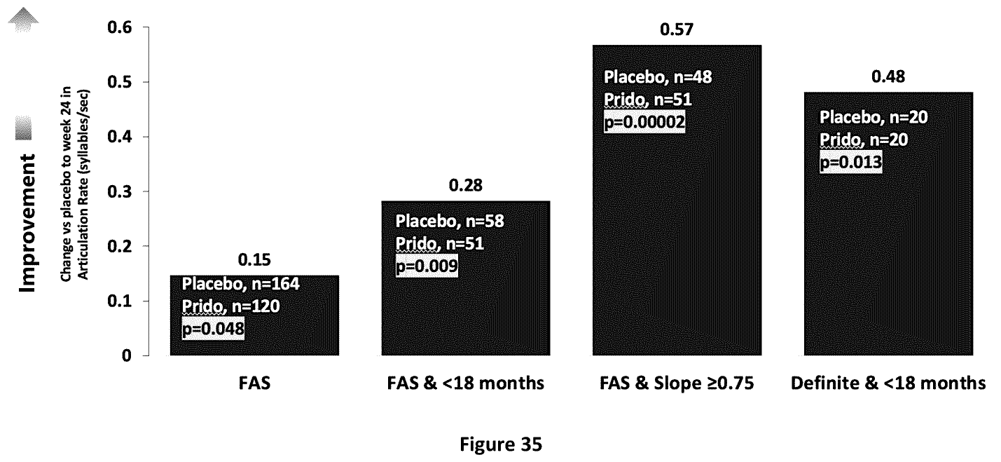

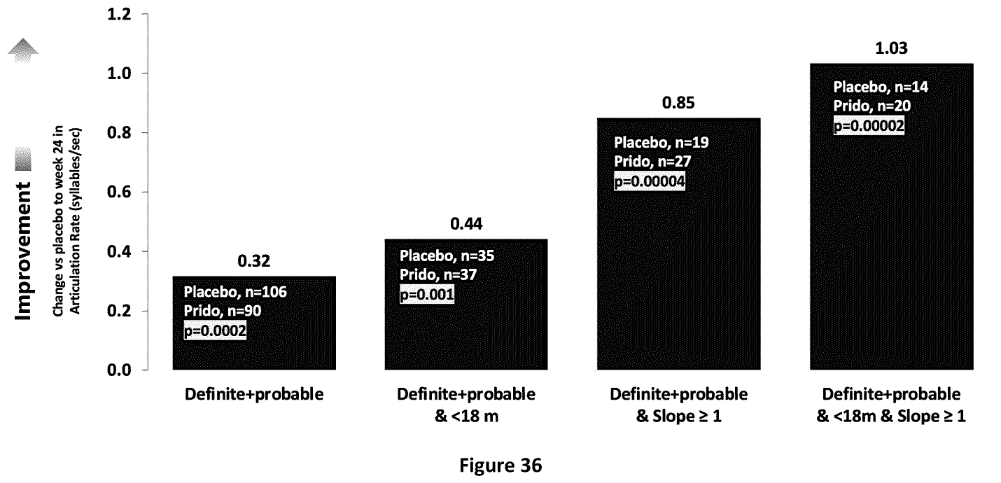

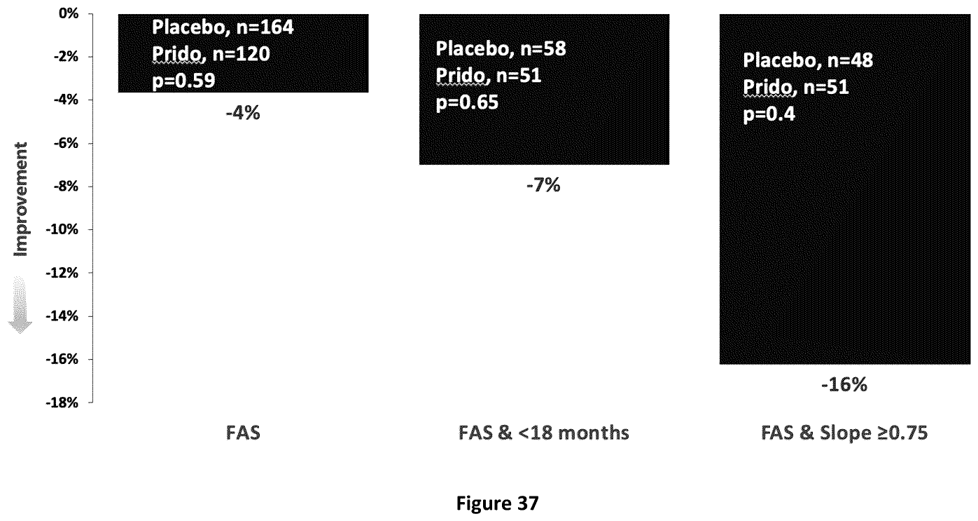

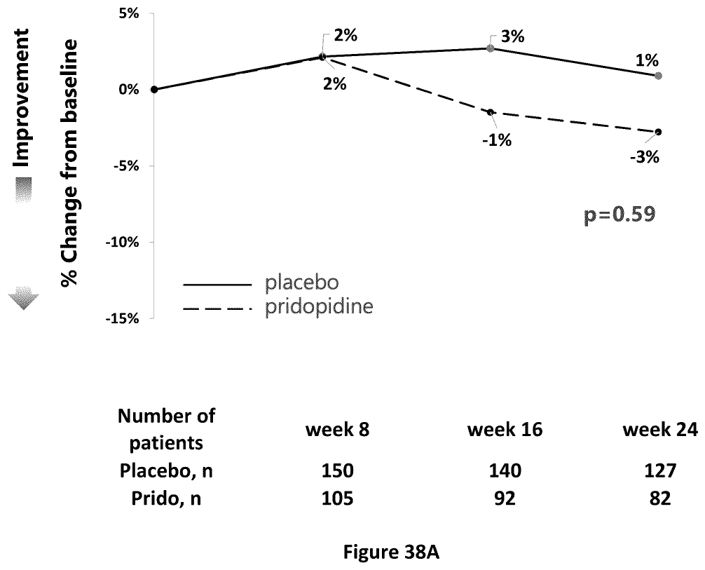

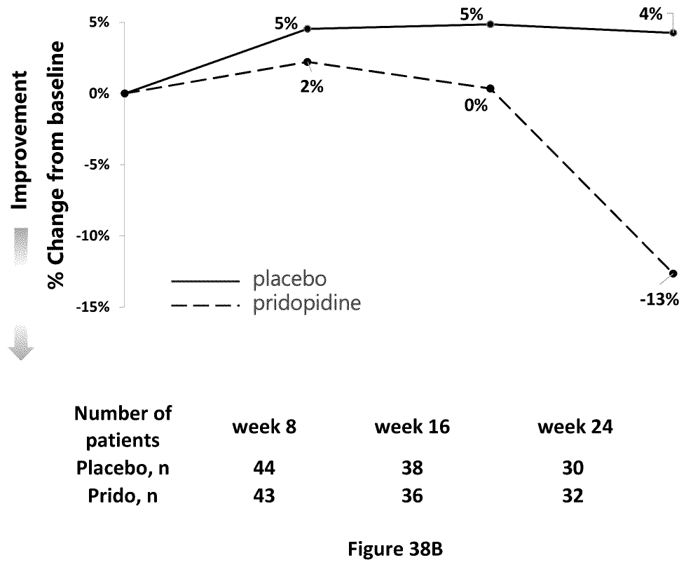

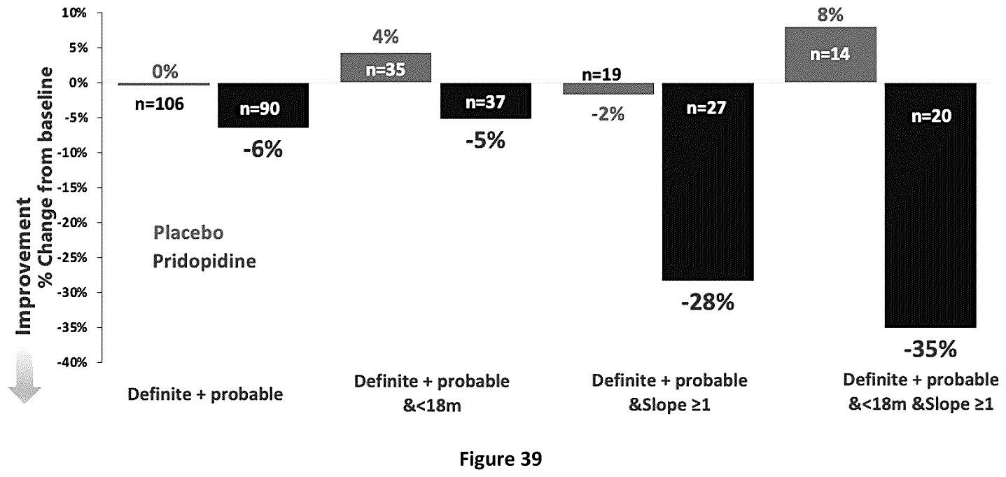

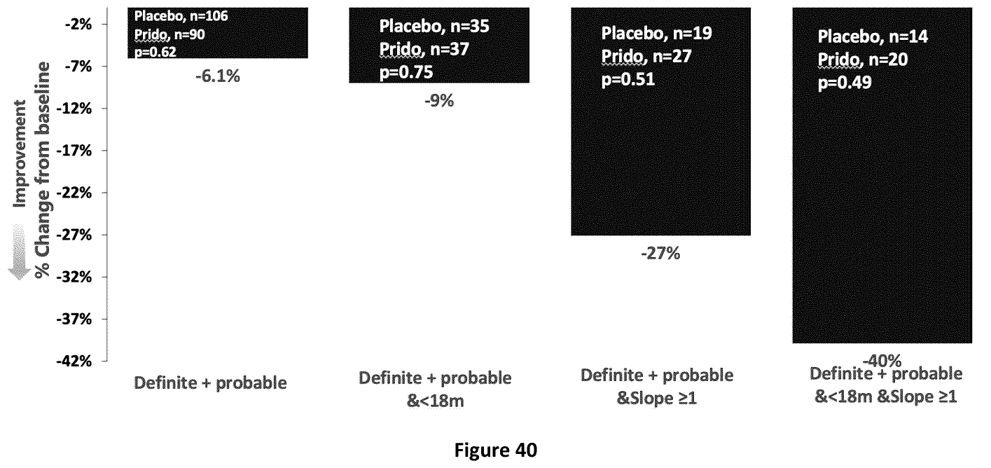

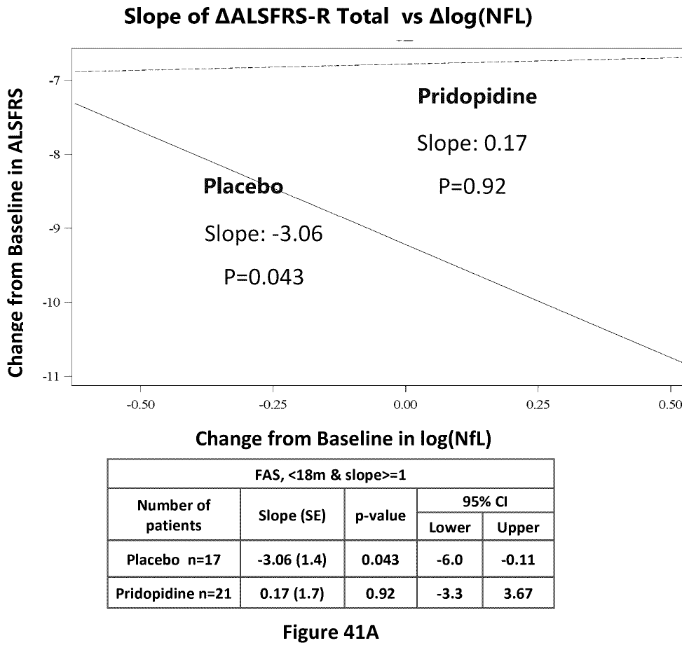

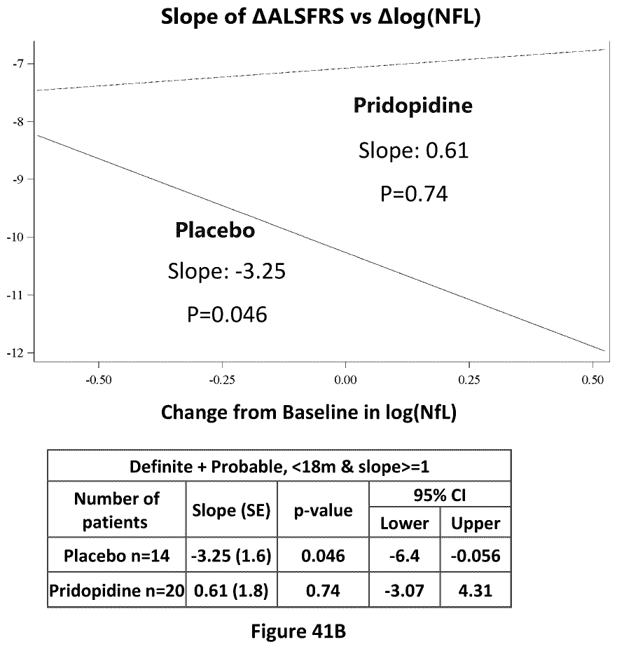

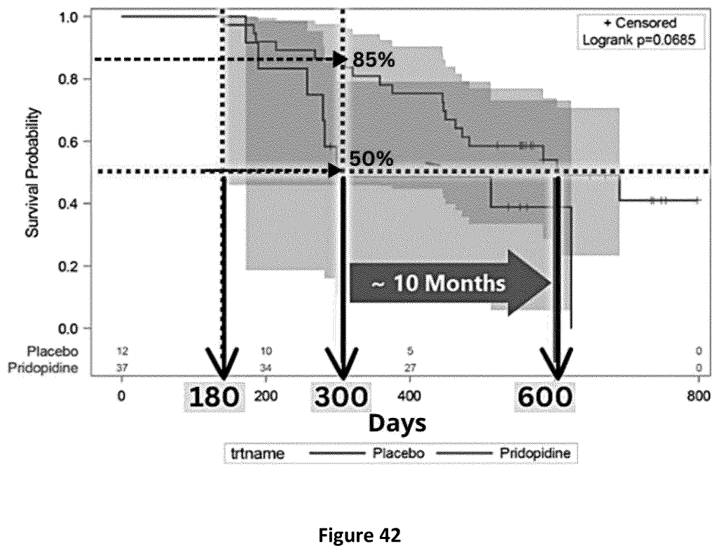

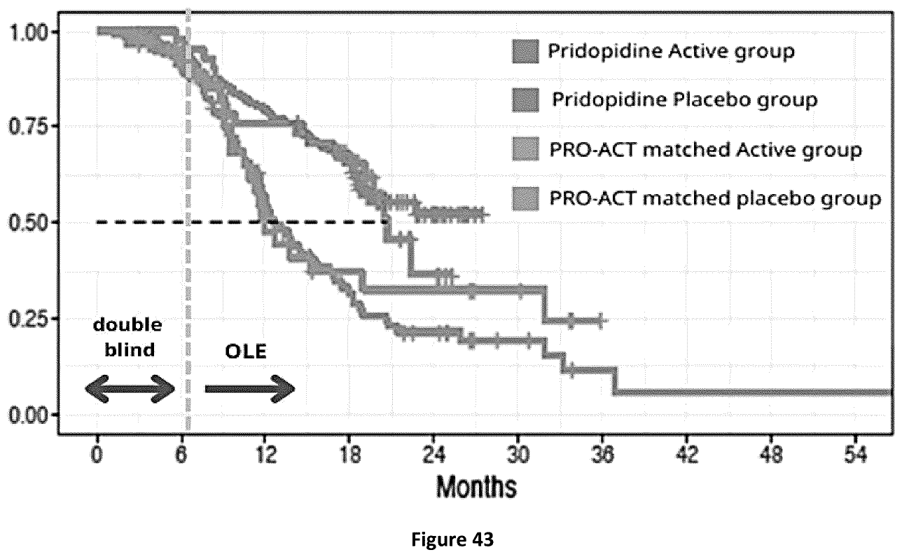

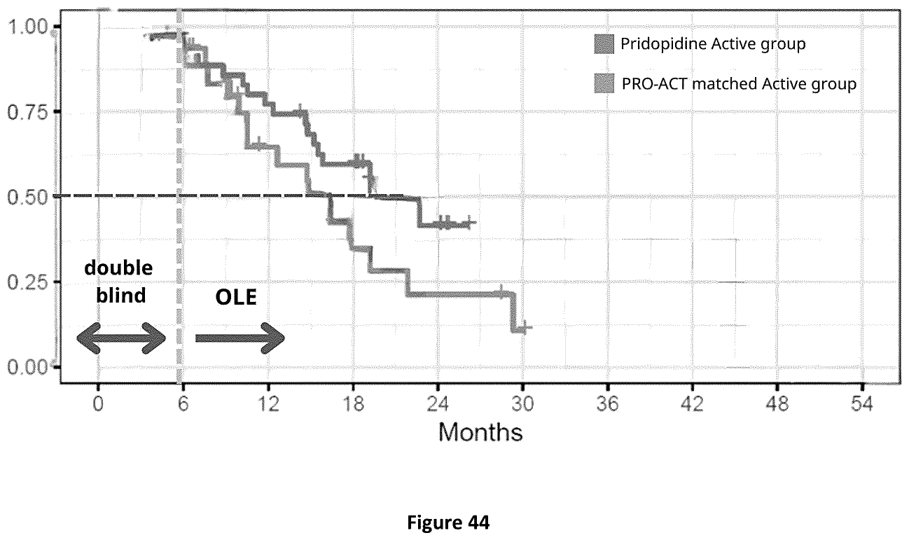

NMJ disruption and the subsequent skeletal muscle wasting are two main pathologies of ALS. The effect of pridopidine on muscle fiber atrophy and preservation of NMJs was evaluated in-vivo. Pre-symptomatic SOD1G93A mice and WT controls (5 weeks old) were treated with either saline as a control, or pridopidine 30 mg/kg, by daily s.c administration for 11 weeks. The Gastrocnemius muscles from vehicle or pridopidine-treated (30 mg/kg s.c.) mice were extracted from the SOD1G93A and WT mice at age 16 weeks. Muscle cross-sections were stained with Hematoxylin & Eosin (H&E), and the mean muscle fiber diameter was quantified for each group (FIG. 10A). NMJ preservation was evaluated by confocal imaging of co-localizing pre (neuronal NFH+Synapsin-I—and post-synaptic (muscular AchR (BTX)) markers and counting the number of fully innervated NMJs in gastrocnemius muscles (FIG. 11A).

### Results

FIG. 10A presents representative images of H&E-stained cross-sections from Gastrocnemius muscle of mice from 3 groups: WT-vehicle treated, SOD1G93A-vehicle treated, and SOD1G93A-30 mg/kg pridopidine treated mice. Muscle histology of SOD1G93A-vehicle mice is poor and reveals a smaller (˜5 μm) diameter of muscle fiber as compared with WT-vehicle (p<0.001) (FIGS. 10A-10B). Pridopidine (30 mg/kg, s.c daily administration) led to a significant ˜4 μm increase in the muscle fiber diameter in SOD1G93A (p<0.05, FIG. 10B).

Muscles of SOD1G93A vehicle-treated mice demonstrated the expected massive ˜60% loss of NMJ and morphological changes in the post-synaptic apparatus mice compared to WT mice (FIGS. 11A-11B). Strikingly, pridopidine treatment limited the loss of NMJs in SOD1G93A mice to ˜20%. Data are shown as mean±SEM. *p-value <0.05; **p-value <0.01; ***p-value <0.001 (double-blind Student's t test).

Overall, these results demonstrate that pridopidine exerted neuroprotective effects in ALS cellular and animal models. In-vitro, in SOD1G93A MNs, pridopidine enhanced BDNF axonal transport, upregulates ERK activation, enhanced axonal growth, restored muscle innervation and improved NMJ formation and function. These neuroprotective effects were mediated by the S1R as a genetic deletion of the S1R gene abolishes pridopidine's effects. In-vivo pridopidine treatment of SOD1G93A ALS mice reduced mutant SOD1 aggregation in the spinal cord (a hallmark of the disease), increased the ALS-reduced muscle fiber diameter and preserved the degenerated NMJs observed in diseased tissue. These data support the use of pridopidine as a neuroprotective agent, and the S1R as a therapeutic target for the treatment of ALS patients.

In the figures, abbreviations are as follows: Geno.=genotype (i.e. wild type (WT), mutant SOD1).

### Experiment 7

### Treatment of ALS in a Human Subject

Periodically orally administering of pridopidine provides a clinically meaningful advantage in reducing the symptoms of ALS in human subjects afflicted with ALS. Pridopidine therapy provides efficacy in treating the patient and is effective in at least one of the following embodiments.
1. The therapy is effective in improving, maintaining, or lessening the decline of symptoms of ALS.
2. The therapy is effective in enhancing BDNF axonal transport in motor neurons and/or enhancing ERK activation.
3. The therapy is effective in improving NMJ formation and preservation, preserving NMJ structure, preserving NMJ function and/or improving innervation rate of muscle tissue.
4. The therapy is effective in enhancing motor neuron axonal growth and/or reducing axonal degeneration, including motor neuron axonal degeneration.
5. The therapy is effective in enhancing muscle cell survival, enhancing muscle fiber diameter and function, reduce progression of muscle fiber wasting, and/or improve muscle contraction; and or
6. The therapy is effective in reducing SOD1 aggregation and/or lessening pseudobulbar disease progression.

In some patients, the attending physician administers pridopidine or pharmaceutically acceptable salt thereof and a second compound, wherein the second compound is riluzole, edaravone, dextromethorphan/quinidine. In some embodiments, the second compound is laquinimod.

### Experiment 8

### Effect of Pridopidine on Motor Neuron Health in TDP43 ΔNLS

Cytoplasmic mislocalization of the RNA-binding protein TDP-43, is reported in >95% of all ALS cases, regardless of the underlying genetic cause. In the inducible transgenic mouse model expressing the human TDP-43 lacking the nuclear-localization-signal (TDP43 ΔNLS), the expression of the truncated protein lacking the NLS is regulated by the doxycycline (DOX) TET-off system. In the presence of DOX, the expression of TDP43 ΔNLS is repressed, and the mouse is healthy. Upon removal of DOX, the truncated protein is expressed and recapitulates ALS-like MN disease pathologies (Walker 2015, Spiller 2016, Spiller 2018).

The effect of pridopidine, and its analogs compound 1 and compound 4 administered individually on neuronal health is evaluated in primary motor neurons (MNs) derived from TDP43 ΔNLS mouse embryos. Neuronal health and survival was assessed by measuring cell cluster area, cell body cluster count, neurite length using high-content image analysis using the Incucyte system. Larger cell clusters and longer neurites are indicative of healthy, active neurons.

### Method

MNs were seeded and maintained in 96-well plate containing 200 μL of with complete Neurobasal (CNB) medium. The MNs were seeded at a density of 10,000 cells per well. The media with the suitable treatment (pridopidine/compound 1/compound 4) was replaced every two days.

Positive control was MNs Treated with Doxycycline (+Dox) in a concentration of 0.1 μg/mL (which don't express human TDP-43 delta NLS), and the negative control was MN without DOX (−Dox, which express human TDP-43 delta NLS).

Cells were automatically imaged at low magnification (20×) by Incucyte Live-Cell Imaging and Analysis instrument at 1 (baseline), 7 and 14 days in vitro (DIV). The images were automatically analyzed by Incucyte software using the neurite tracking function to analyze the cell body cluster area (area/mm2), cell body cluster count (per mm2) and neurite length (mm/mm2). All assays were performed in quintuplicate in 3 independent experiments. Data was normalized to the +Dox (positive control) condition, and compared to −Dox (negative control) condition Using One-way ANOVA test. P-values: *p<0.05, **p<0.01, ***p<0.001, ****p<0.0001)

### Results

Cells expressing the truncated protein TDP43 ΔNLS demonstrate ˜30% decreased cell cluster area, cell cluster body number and neuritic length. Pridopidine treatment rescues cell cluster area (FIG. 12), cell cluster count (FIG. 13), and neuritic length (FIGS. 14A-14B) back to control levels. Similarly, compound 1 treatment rescues cell cluster area (FIGS. 15A-15B), cell cluster count (FIGS. 16A-16B), and neuritic length (FIGS. 17A-17B) back to control levels. Compound 4 treatment also rescues cell cluster area (FIGS. 18A-18B), cell cluster count (FIGS. 19A-19B), and neuritic length (FIGS. 20A-20B) back to control levels.

### Experiment 9

### ALS Clinical Trial

The ALS Platform Trial is managed by the Healey Center for ALS at the Massachusetts General Hospital. This was a multicenter, multi-regimen, randomized, placebo-controlled, adaptive platform clinical trial evaluating the safety and efficacy of multiple investigational products simultaneously or sequentially in ALS.

Treatment duration of placebo-controlled regimens was a maximum of 24-weeks for each regimen. An optional open label extension (OLE) may be offered.

The specific pridopidine regimen was based on cumulative preclinical and clinical studies that suggest a beneficial effect for pridopidine in ALS. Pridopidine acts primarily as a Sigma-1 Receptor (S1R) agonist.

The purpose of this clinical study of pridopidine was to evaluate the effect of pridopidine 45 mg BID on ALS disease progression including functional decline, bulbar function, muscle strength, function of upper and lower limb, voice and speech characteristics, respiratory function and biomarker levels in participants with ALS.

The number of planned participants for the pridopidine regimen is 160.

There were 2 treatment groups for this regimen, active and placebo. Participants were randomized in a 3:1 ratio to active treatment or placebo (i.e., 120 active: 40 placebo).

The maximum duration of the placebo-controlled treatment period was 24 weeks. Placebo was shared from 4 regimens in the trial, with a total of 164 subjects on Placebo and 120 subjects on pridopidine.

### Dosing Regimen

45 mg pridopidine was administered twice daily (BID), taken in the morning and in the early afternoon (approximately 7 to 10 hours after the morning dose).

There was a titration period leading up to the proposed dose whereby participants were initiating pridopidine at 45 mg QD and then increasing to 45 mg BID after 2 weeks.

### Inclusion Criteria

1. Sporadic or familial ALS diagnosed as clinically possible, probable, lab-supported probable, or definite ALS defined by revised El Escorial criteria (Appendix I).

2. Age 18 years or older.

3. Capable of providing informed consent and complying with study procedures, in the SI's opinion.

4. Time since onset of weakness due to ALS≤36 months at the time of the Master Protocol Screening Visit.

5. Vital Capacity ≥50% of predicted capacity for age, height, and sex at the time of the Master Protocol Screening Visit measured by Slow Vital Capacity (SVC), or, if required due to pandemic-related restrictions, Forced Vital Capacity (FVC) measured in person or via telemedicine, or sustained phonation.

6. Participants must either not take riluzole or be on a stable dose of riluzole for ≥30 days prior to the Master Protocol Screening Visit. Riluzole-naïve participants are permitted in the study.

7. Participants must either not take edaravone or have completed at least one cycle of edaravone prior to the Master Protocol Screening Visit. Edaravone-naïve participants are permitted in the study.

8. Participants must have the ability to swallow pills and liquids at the time of the Master Protocol Screening Visit and, in the SI's opinion, have the ability to swallow for the duration of the study.

### Study Objectives

### Primary Efficacy Objective:

To evaluate the efficacy of pridopidine as compared to placebo on ALS disease progression.

### Secondary Efficacy Objective;

To evaluate the effect of pridopidine on selected secondary measures of disease progression, including survival.

### Safety Objective:

To evaluate the safety of pridopidine in ALS patients.

### Exploratory Efficacy Objective:

To evaluate the effect of pridopidine on selected biomarkers and endpoints.

### Study Endpoints

### Primary Efficacy Endpoint:

Change in disease severity as measured by the ALS Functional Rating Scale-Revised (ALSFRS-R) using a Bayesian repeated measures model that accounts for loss to follow-up due to mortality.

### Justification—

The ALSFRS-R measures function in daily activities and is an established scale for monitoring disease progression in ALS. Each type of function is scored from 4 (normal) to 0 (no ability), with a maximum total score of 48 and a minimum total score of 0. Patients with higher scores have more physical function.

### Secondary Efficacy Endpoints:

Bulbar Function in Participants with Bulbar Dysfunction

Rate of change in ALSFRS-R bulbar subdomain (Q1-Q3) score among participants with bulbar dysfunction at baseline, each question is scored from 4 (normal) to 0 (no ability), with a maximum total score of 12 and a minimum total score of 0 for the bulbar subdomain. Patients with higher scores have more bulbar function.

### Bulbar Function in all Randomized Participants

Rate of change in ALSFRS-R bulbar subdomain (Q1-Q3) score among all randomized participants. Each question is scored from 4 (normal) to 0 (no ability), with a maximum total score of 12 and a minimum total score of 0 for the bulbar subdomain. Patients with higher scores have more bulbar function.

### Speech

Rate of change in the speech sub-score of the ALSFRS-R (Q1) among all randomized participants. The speech question is scored from 4 (normal) to 0 (no ability), with a maximum total score of 4 and a minimum total score of 0. Patients with higher scores have better speech.

### Respiratory Function

Rate of change in SVC maximum percent of predicted among all randomized participants,

Bulbar Function in Participants with Rapid Pre-Baseline Progression

Rate of change in ALSFRS-R bulbar subdomain (Q1-Q3) score among participants with pre-baseline slope ≥0.75 points/month, each question is scored from 4 (normal) to 0 (no ability), with a maximum total score of 12 and a minimum total score of 0 for the bulbar subdomain. Patients with higher scores have more bulbar function.

### Time to Bulbar Dysfunction

Time from baseline to the first observed bulbar dysfunction as measured by an ALS Functional Rating Scale-Revised (ALSFRS-R) bulbar subdomain score of less than 12.

### Muscle Strength

Rate of change in muscle strength as measured isometrically using hand-held dynamometry HHD.

Percent change from baseline among all randomized participants,

### Survival

Time to death or death equivalent.

### Other Secondary Efficacy Endpoints (Pre-Specified Non Multiplicity Adjusted Non-Hierarchical)

Time to first decline of 2 points or greater post baseline in the ALSFRS-R total score among participants in FAS,
Proportion of participants experiencing 5 points or less decline in the ALSFRS-R total score from baseline through Week 24 among all participants in FAS,
Proportion of participants experiencing 5 points or less decline in the ALSFRS-R total score from baseline through Week 24 among all participants in FAS with baseline ALSFRS-R greater than or equal to 36 (Early in disease),
Proportion of participants experiencing 5 points or less decline in the ALSFRS-R total score from baseline through Week 24 among all participants in FAS with bulbar dysfunction at baseline defined as an ALSFRS-R bulbar domain (Q1-Q3) score of less than 12,
Rate of change in ALSFRS-R Total Score from baseline through Week 24 among participants in FAS with bulbar dysfunction at baseline defined as an ALSFRS-R bulbar domain (Q1-Q3) score of less than 12,
Proportion of participants experiencing no or only a 1 point decline in the ALSFRS-R bulbar domain (Q1-Q3) score from baseline through Week 24 among participants in FAS with bulbar dysfunction at baseline defined as an ALSFRS-R bulbar domain (Q1-Q3) score of less than 12,
Time to first decline of 1 or more points post baseline in the ALSFRS-R bulbar domain (Q1-Q3) score among participants in FAS with bulbar dysfunction at baseline defined as an ALSFRS-R bulbar domain (Q1-Q3) score of less than 12,
Proportion of participants experiencing no worsening in the ALSFRS-R bulbar domain (Q1-Q3) score from baseline through Week 24 among all participants in FAS with delta-FRS slower than-0.75 points/month (slow progressors),
Proportion of participants with ALSFRS-R bulbar domain (Q1-Q3) score greater than or equal to 9 (out of 12) at Week 24 among all participants in FAS with bulbar dysfunction at baseline defined as an ALSFRS-R bulbar domain (Q1-Q3) score of less than 12,
Proportion of participants experiencing no worsening in the ALSFRS-R bulbar domain (Q1-Q3) score from baseline through Week 24 among all participants in FAS with baseline ALSFRS-R greater than or equal to 36 (Early in disease),
Rate of change in CNS-BFS from baseline through Week 24 among all participants in FAS,
Rate of change in CNS-BFS from baseline through Week 24 among participants in FAS with bulbar dysfunction at baseline defined as an ALSFRS-R bulbar domain (Q1-Q3) score of less than 12,
Change in HHD upper extremity percentage from baseline through Week 24
Change in HHD lower extremity percentage from baseline through Week 24,
Change in log transformed NfL from baseline through Week 24 among all participants in FAS,
Change in log transformed NfL from baseline through Week 24 among participants in FAS with bulbar dysfunction at baseline defined as an ALSFRS-R bulbar domain (Q1-Q3) score of less than 12,
Change in log transformed NfL from baseline through Week 24 among participants in FAS by NfL Median baseline split (slow vs fast progressors),
Rate of change in ALSFRS-R total score from baseline through Week 24 among participants in FAS who were not on Nuedexta at baseline,
Rate of change in ALSFRS-R total score from baseline through Week 24 among participants in FAS who were not on Nuedexta at baseline and with bulbar dysfunction at baseline defined as an ALSFRS-R bulbar domain (Q1-Q3) score of less than 12,
Rate of change in CNS-BFS from baseline through Week 24 among participants in FAS who were not on Nuedexta at baseline,
Rate of change in CNS-BFS from baseline through Week 24 among participants in FAS who were not on Nuedexta at baseline and with bulbar dysfunction at baseline defined as an ALSFRS-R bulbar domain (Q1-Q3) score of less than 12,
Proportion of participants experiencing no worsening in the CNS-BFS from baseline through Week 24 among all participants in FAS who were not on Nuedexta at baseline,
Time to first increase of 5 points or more post baseline in the CNS-BFS score among all participants in FAS who were not on Nuedexta at baseline, and
Rate of change in ALSFRS-R total score from baseline through Week 24 among participants in FAS who were on Nuedexta at baseline.
   Exploratory Endpoints—the Following Categories of Exploratory Endpoints were Evaluated:
Rate of change in ALSFRS-R bulbar domain (Q1-Q3) score from baseline through Week 24 among participants in FAS,
Rate of change in ALSFRS-R bulbar domain (Q1-Q3) score from baseline through Week 24 among participants in FAS with bulbar dysfunction at baseline defined as an ALSFRS-R bulbar domain (Q1-Q3) score of less than 12,
Proportion of participants experiencing no worsening in the ALSFRS-R bulbar domain (Q1-Q3) score from baseline through Week 24 among all participants in FAS with delta-FRS less than −0.75 points/month (fast progressors),
Rate of change in ALSFRS-R bulbar domain (Q1-Q3) score from baseline through Week 24 among all participants in FAS with delta-FRS greater than −0.75 points/month (slow progressors),
Decline in respiratory function is a direct result of the known pathophysiology of the ALS and demonstration of a treatment benefit on respiratory endpoints may also provide evidence of effectiveness.

Loss of strength is a hallmark of disease progression in ALS and meaningful differences in muscle strength should be supportive of an effect on measures of function in activities of daily living.

### Additional Exploratory Endpoints

Changes in quantitative voice characteristics.
Changes in biofluid biomarkers of neurodegeneration.
Changes in patient reported outcomes.

These endpoints provide greater understanding of ALS and may provide identification of surrogate endpoints that are reasonably likely to predict clinical benefit.

### Methods

ALS Functional Rating Scale—Revised (ALSFRS-R), is a quickly administered (5 minutes) ordinal rating scale used to determine participants' assessment of their capability and independence in 12 functional activities. Each functional activity is rated 0-4 for a total score that ranges from 0 to 48. Higher scores indicate better function. Initial validity in ALS patients was established by documenting that, change in ALSFRS-R scores correlated with change in strength over time, was closely associated with quality-of-life measures, and predicted survival. The test-retest reliability is greater than 0.88 for all test items. The advantages of the ALSFRS-R are that all 12 functional activities are relevant to ALS, it is a sensitive and reliable tool for assessing activities of daily living function in those with ALS, and it is quickly administered. With appropriate training the ALSFRS-R can be administered with high inter-rater reliability and test-retest reliability. The ALSFRS-R can be administered by phone with good inter-rater and test-retest reliability. The equivalency of phone versus in-person testing, and the equivalency of study participant versus caregiver responses have also been established. Additionally, the ALSFRS-R can also be obtained using a web-based interface with good concordance with in-person assessment. All ALSFRS-R evaluators must be NEALS certified.

Slow Vital Capacity (SVC), the vital capacity (VC) is determined using the upright slow VC method. All VC evaluators must be NEALS certified. The VC is measured using the Easyone Air spirometer, and assessments is performed using a face mask. A printout from the spirometer of all VC trials will be retained. Three VC trials are required for each testing session, however up to 5 trials may be performed if the variability between the highest and second highest VC is 10% or greater for the first 3 trials. Only the 3 best trials were recorded on the CRF. The highest VC recorded was utilized for eligibility. At least 3 measurable VC trials were completed to score VC for all visits after screening. Predicted VC values and percent-predicted VC values were calculated using the Quanjer Global Lung Initiative equations.

### Measures of Muscle Strength

Handheld Dynamometry: HHD is used as a quantitative measure of muscle strength for this study. Six proximal muscle groups were examined bilaterally in both upper and lower extremities (shoulder flexion, elbow flexion, elbow extension, hip flexion, knee flexion, and knee extension), all of which have been validated against maximum voluntary isometric contraction (MVIC) testing 19. In addition, wrist extension, abductor pollicis brevis, abductor digiti minimi, first dorsal interosseous contraction and ankle dorsiflexion were measured bilaterally; these muscles are often affected in ALS.

Bilateral Hand Grip: Bilateral hand grip were measured using a Jamar hand dynamometer to test the maximum isometric strength of the hand and forearm muscles, measured in pounds.

Voice Analysis. In addition to the scheduled in clinic voice recordings, voice samples were collected twice per week and at each in person visit, using an app installed on either an android or iOS-based smartphone. The app characterizes ambient noise, then asks participants to perform a set of speaking tasks: reading sentences—5 fixed and 5 chosen at random from a large sentence bank—repeating a consonant-vowel sequence, producing a sustained phonation, and counting on a single breath. Voice signals were uploaded to a HIPAA-compliant web server, where an AI-based analysis identifies relevant vocal attributes. Quality control (QC) of individual samples occurred by evaluation of voice records by trained personnel.

The goal of using quantitative voice analysis in the Healey Platform trial was to provide a more sensitive and accurate tool for evaluating the progression of ALS and to monitor the efficacy of treatments for the disease.

In the trial, participants were asked to repeat a set of standardized speech tasks and their speech was recorded and analyzed using quantitative voice analysis technology. The resulting data was used to quantify changes in speech parameters, such as speech rate, pitch, and loudness, which are known to be affected by ALS. The data was then used to evaluate the progression of the disease and the impact of treatments on disease progression.

Center for Neurologic Study Bulbar Function Scale. The Center for Neurologic Study Bulbar Function Scale (CNS-BFS) is a participant self-report scale that has been developed for use as an endpoint in clinical trials and as a clinical measure for evaluating and following ALS patients (Smith et al, 2018). The CNS-BFS consists of three domains (swallowing, speech, and salivation), which are assessed with a 21-question, self-report questionnaire. Higher scores indicate greater bulbar dysfunction. Participants will be handed the questionnaire and asked to write their answers themselves. Caregivers can also help, if needed. Instructions on administering the questionnaire during a phone or telemedicine visit were included in the MOP.

CNS-Lability Scale, the Center for Neurologic Study Lability Scale (CNS-LS) is a participant self-report scale that has been developed for use as an endpoint in clinical trials and as a clinical measure for evaluating emotional lability. The CNS-LS is a short (seven-question), self-report questionnaire, designed to be completed by the participant, that provides a quantitative measure of the perceived frequency of PBA episodes. Higher scores indicate greater emotional lability. A CNS-LS score of 13 or higher may suggest PBA. For all in person visits, participants were handed the questionnaire and asked to write their answers themselves. Caregivers can also help, if needed. During telephone visits, site staff were administer and record data for this scale.

ALSAQ-40. The Amyotrophic Lateral Sclerosis Assessment Questionnaire-40 (ALSAQ-40) is a participant self-report health status patient-reported outcome. The ALSAQ-40 consists of forty questions that are specifically used to measure the subjective well-being of participants with ALS and motor neuron disease. Higher scores indicate a decrease in quality of life. Participants were handed the questionnaire and asked to write their answers themselves. Caregivers can also help, if needed.

### Results

The Results of this Experiment are Detailed in FIGS. 21-41 and Below.

“Pridopidine” as disclosed herein refers to “pridopidine hydrochloride”.

### Pridopidine Demonstrates Less Decline Vs Placebo on Disease Progression Assessed by the ALSFRS-R Total Scale

The effect of pridopidine on disease progression was assessed using the ALSFRS-R Total scale, and its respiratory and bulbar sub-scales. ALSFRS-R data was collected at baseline, week 8, week 16 and 24 weeks. The change from baseline at each visit were calculated and compared between the pridopidine and placebo groups using both the Random Slopes Statistical model and the MMRM statistical Model.

Participating subjects were classified by time from symptom onset (<18 months was the cutoff), faster progression, defined by pre-baseline ALSFRS-R slope (either ≥0.75 or ≥1), and El Escorial criteria of definite and/or probable ALS.

Pridopidine demonstrates a beneficial effect on ALSFRS-R compared to placebo (FIG. 21). This effect is enhanced in subjects with pre-baseline slope of ≥0.75 and in subjects with symptom onset <18 months. The greatest effect is observed in subjects with definite ALS <18 months from symptom onset.

The beneficial effect of pridopidine showing less decline vs placebo in ALSFRS-R is larger in definite+probable ALS subjects (FIG. 22). Among definite+probable subjects, pridopidine demonstrates greater, statistically significant effects in patients <18 months from symptom onset (change vs. placebo 2.9, p=0.03, positive change indicates improvement) and subjects with <18 months from symptom onset and pre-baseline ALSFRS-R slope ≥1 (change vs. placebo 5.2, p=0.04). An improvement is also observed in definite+probable subjects with pre-baseline ALSFRS-R slope ≥1 (change vs. placebo 3.4, p=0.07).

Time-course analysis of the effect of pridopidine and placebo on ALSFRS-R demonstrates that pridopidine mitigates the decline in ALSFRS-R from week 8 (FIGS. 23A-23C). The effect is most pronounced in definite ALS subjects <18 months from symptom onset (see Table 1). In the full analysis set (FAS), subjects <18 months from symptom onset and pre-baseline slope >=1 pridopidine demonstrates a trend towards reducing the decline vs placebo at weeks 8 and 16. The effect is largest at week 24 (change vs. placebo 4.19, p=0.07) (FIG. 24A). In definite+probable ALS subjects <18 months from symptom onset and pre-baseline slope >=1, the effect is larger and statistically at all timepoints (FIG. 24B, Table 1).

TABLE 1
Pridopidine shows less decline vs placebo in ASLFRS-R Total Score in ALS subjects.
Change from baseline to week 8, 16 and 24 in different groups.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 155 | −2.01 | 0.2292 | 112 | −1.69 | 0.2681 | 0.32 | 0.3672 | 0.3672 |
| | 16 | 148 | −4.22 | 0.298 | 104 | −3.73 | 0.3512 | 0.49 | 0.2829 | 0.2829 |
| | 24 | 143 | −5.99 | 0.3745 | 99 | −5.74 | 0.4431 | 0.25 | 0.6696 | 0.6696 |
| FAS + Symptom | 8 | 54 | −2.55 | 0.3724 | 49 | −1.59 | 0.3928 | 0.96 | 0.0819 | 0.0819 |
| Onset <18 Months | 16 | 52 | −5.33 | 0.5495 | 47 | −3.61 | 0.5798 | 1.72 | 0.0367 | 0.0367 |
| | 24 | 49 | −7.23 | 0.6913 | 46 | −5.82 | 0.7243 | 1.41 | 0.1677 | 0.1677 |
| FAS + ALSFRS-R | 8 | 20 | −3.83 | 0.8035 | 27 | −2.04 | 0.6847 | 1.79 | 0.0987 | 0.0987 |
| Pre baseline | 16 | 17 | −7.79 | 1.1295 | 26 | −5.05 | 0.9451 | 2.74 | 0.0697 | 0.0697 |
| slope >=1 | 24 | 16 | −9.99 | 1.3278 | 24 | −7.57 | 1.1068 | 2.42 | 0.1686 | 0.1686 |
| FAS + <18 & re | 8 | 14 | −4.63 | 1.0123 | 20 | −1.81 | 0.8511 | 2.82 | 0.043 | 0.043 |
| baseline slope >=1 | 16 | 12 | −9.15 | 1.474 | 20 | −4.68 | 1.2108 | 4.47 | 0.0267 | 0.0267 |
| | 24 | 11 | −11.33 | 1.699 | 19 | −7.14 | 1.3814 | 4.19 | 0.0663 | 0.0663 |
| Definite | 8 | 61 | −2.32 | 0.3678 | 46 | −1.91 | 0.4205 | 0.41 | 0.4742 | 0.4742 |
| | 16 | 56 | −5.28 | 0.4741 | 42 | −4.45 | 0.5485 | 0.83 | 0.2635 | 0.2635 |
| | 24 | 56 | −7.58 | 0.5985 | 38 | −6.59 | 0.7002 | 0.99 | 0.296 | 0.296 |
| Definite + Onset | 8 | 18 | −2.9 | 0.5424 | 19 | −2.48 | 0.5224 | 0.42 | 0.5962 | 0.5962 |
| <18 Months | 16 | 16 | −6.76 | 0.9363 | 20 | −4.92 | 0.8588 | 1.84 | 0.1729 | 0.1729 |
| | 24 | 16 | −9.81 | 1.2755 | 19 | −7.4 | 1.1875 | 2.41 | 0.1904 | 0.1904 |
| Probable | 8 | 40 | −2.73 | 0.5413 | 37 | −1.34 | 0.5453 | 1.39 | 0.0758 | 0.0758 |
| | 16 | 40 | −4.96 | 0.6314 | 37 | −2.79 | 0.641 | 2.17 | 0.0196 | 0.0196 |
| | 24 | 36 | −6.49 | 0.6989 | 35 | −4.45 | 0.7105 | 2.04 | 0.0468 | 0.0468 |
| Definite + Probable | 8 | 101 | −2.41 | 0.3078 | 83 | −1.76 | 0.3333 | 0.65 | 0.1539 | 0.1539 |
| | 16 | 96 | −5.07 | 0.3826 | 79 | −3.85 | 0.417 | 1.22 | 0.0327 | 0.0327 |
| | 24 | 92 | −7.03 | 0.461 | 73 | −5.79 | 0.5068 | 1.24 | 0.074 | 0.074 |
| Definite + Probable & | 8 | 33 | −3.25 | 0.5406 | 36 | −1.71 | 0.5134 | 1.54 | 0.0446 | 0.0446 |
| Onset <18 months | 16 | 31 | −6.7 | 0.781 | 36 | −3.68 | 0.7358 | 3.02 | 0.0069 | 0.0069 |
| | 24 | 29 | −9 | 0.9375 | 35 | −6.1 | 0.8742 | 2.9 | 0.0282 | 0.0282 |
| Definite + Probable & | 8 | 17 | −4.36 | 0.9193 | 25 | −2.21 | 0.7351 | 2.15 | 0.0783 | 0.0783 |
| Pre baselineslope >=1 | 16 | 15 | −8.66 | 1.2532 | 24 | −5.21 | 0.9943 | 3.45 | 0.0386 | 0.0386 |
| | 24 | 14 | −10.96 | 1.421 | 22 | −7.56 | 1.1318 | 3.4 | 0.0707 | 0.0707 |
| Definite + Probable & | 8 | 12 | −5.42 | 1.1278 | 19 | −1.94 | 0.8785 | 3.48 | 0.0235 | 0.0235 |
| Onset <18 months & | 16 | 10 | −10.41 | 1.6303 | 19 | −4.97 | 1.2421 | 5.44 | 0.0139 | 0.0139 |
| pre-baseline | 24 | 9 | −12.71 | 1.8936 | 18 | −7.51 | 1.4264 | 5.2 | 0.0384 | 0.0384 |
| slope >=1 | | | | | | | | | | |

### Pridopidine Demonstrates Beneficial Effects on Respiratory Functions

The effect of pridopidine on respiratory function was assessed using the ALSFRS-R Respiratory sub-scale. In the FAS, pridopidine demonstrates less decline vs placebo in respiratory function (change vs. placebo 0.09, p=0.06). The effect is larger in subjects with faster progression with a pre-baseline slope ≥0.75 (change vs. placebo 0.11, p=0.26), and subjects who are early with symptom onset <18 months (change vs, placebo 0.11, p=0.14). The effect is largest in definite ALS subjects <18 months from symptom onset (change vs. placebo 0.2, p=0.12) (FIG. 25, Random Slopes Model and Table 2).

The beneficial effect of pridopidine on respiratory function is also demonstrated when analyzed using the MMRM statistical model. In the FAS, pridopidine demonstrated a beneficial effect vs placebo (change vs. placebo 0.44, p=0.09), which was greater in subjects with faster progression having pre-baseline slope ≥0.75 (change vs, baseline 0.53, p=0.34), early with <18 months from symptom onset (change vs. baseline 0.79, p=0.08) and definite ALS subjects early with <18 months from symptom onset (change vs. placebo 1.04, p=0.18) (FIG. 26 and Table 2).

Time-course analysis demonstrates that pridopidine shows less decline vs placebo in ALSFRS-R respiratory score from week 8 in FAS and FAS subjects who are early with <18 months from symptom onset, and at 16 weeks in FAS who are faster progressors with pre-baseline slope ≥0.75, and definite ALS subjects who are early with <18 months from symptom onset (FIGS. 27A-27D and Table 2).

TABLE 2
Pridopidine shows less decline vs placebo in ASLFRS-R Respiratory in ALS subjects.
Change from baseline to week 8, 16 and 24 in different groups.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 155 | −0.38 | 0.121 | 112 | −0.3 | 0.1416 | 0.08 | 0.1871 | 0.6631 |
| | 16 | 148 | −0.84 | 0.1475 | 104 | −0.52 | 0.174 | 0.32 | 0.2294 | 0.1626 |
| | 24 | 143 | −1.24 | 0.1657 | 99 | −0.79 | 0.1963 | 0.45 | 0.2583 | 0.0877 |
| FAS + Symptom | 8 | 54 | −0.66 | 0.1907 | 49 | −0.2 | 0.2012 | 0.46 | 0.2799 | 0.1061 |
| Onset <18 Months | 16 | 52 | −1.31 | 0.2648 | 47 | −0.51 | 0.2798 | 0.8 | 0.3898 | 0.0407 |
| | 24 | 49 | −1.61 | 0.3034 | 46 | −0.83 | 0.3172 | 0.78 | 0.444 | 0.0789 |
| FAS + ALSFRS-R | 8 | 20 | −0.89 | 0.4676 | 27 | −0.62 | 0.3965 | 0.27 | 0.6153 | 0.6623 |
| Pre baselineslope >=1 | 16 | 17 | −1.59 | 0.5417 | 26 | −0.86 | 0.4516 | 0.73 | 0.7073 | 0.3066 |
| | 24 | 16 | −1.98 | 0.6225 | 24 | −1.34 | 0.5173 | 0.64 | 0.8131 | 0.4369 |
| FAS + <18 & re | 8 | 14 | −1.15 | 0.5567 | 20 | −0.38 | 0.467 | 0.77 | 0.731 | 0.2967 |
| baseline slope >=1 | 16 | 12 | −2.04 | 0.6445 | 20 | −0.59 | 0.527 | 1.45 | 0.8372 | 0.0957 |
| | 24 | 11 | −2.27 | 0.7453 | 19 | −0.99 | 0.6019 | 1.28 | 0.9645 | 0.1969 |
| Definite | 8 | 61 | −0.31 | 0.2091 | 46 | −0.47 | 0.2397 | −0.16 | 0.3221 | 0.6209 |
| | 16 | 56 | −0.85 | 0.2378 | 42 | −0.65 | 0.2745 | 0.2 | 0.3693 | 0.5881 |
| | 24 | 56 | −1.48 | 0.2806 | 38 | −0.99 | 0.3303 | 0.49 | 0.4409 | 0.2698 |
| Definite + Onset | 8 | 18 | −0.37 | 0.2651 | 19 | −0.44 | 0.2544 | −0.07 | 0.3863 | 0.8539 |
| <18 Months | 16 | 16 | −1.16 | 0.4225 | 20 | −0.68 | 0.3879 | 0.48 | 0.5977 | 0.4318 |
| | 24 | 16 | −1.98 | 0.5477 | 19 | −0.94 | 0.4963 | 1.04 | 0.7654 | 0.1843 |
| Probable | 8 | 40 | −0.76 | 0.292 | 37 | −0.11 | 0.2943 | 0.65 | 0.4179 | 0.1221 |
| | 16 | 40 | −1.4 | 0.3274 | 37 | −0.13 | 0.3327 | 1.27 | 0.4701 | 0.0086 |
| | 24 | 36 | −1.45 | 0.3422 | 35 | −0.27 | 0.3464 | 1.18 | 0.4908 | 0.0188 |
| Definite + Probable | 8 | 101 | −0.46 | 0.1696 | 83 | −0.35 | 0.184 | 0.11 | 0.2517 | 0.6549 |
| | 16 | 96 | −1.05 | 0.1949 | 79 | −0.46 | 0.2121 | 0.59 | 0.2899 | 0.046 |
| | 24 | 92 | −1.45 | 0.2163 | 73 | −0.71 | 0.2379 | 0.74 | 0.3238 | 0.0248 |
| Definite + Probable & | 8 | 33 | −0.8 | 0.2534 | 36 | −0.23 | 0.2407 | 0.57 | 0.3518 | 0.1158 |
| Onset <18 months | 16 | 31 | −1.57 | 0.3503 | 36 | −0.33 | 0.3297 | 1.24 | 0.4852 | 0.013 |
| | 24 | 29 | −1.94 | 0.3948 | 35 | −0.74 | 0.3665 | 1.2 | 0.5432 | 0.0304 |
| Definite + Probable & | 8 | 17 | −1.13 | 0.5202 | 25 | −0.67 | 0.414 | 0.46 | 0.6681 | 0.4933 |
| Pre baseline | 16 | 15 | −1.85 | 0.6007 | 24 | −0.84 | 0.4755 | 1.01 | 0.7688 | 0.1958 |
| slope >=1 | 24 | 14 | −2.4 | 0.6695 | 22 | −1.3 | 0.5309 | 1.1 | 0.862 | 0.2077 |
| Definite + Probable & | 8 | 12 | −1.46 | 0.5894 | 19 | −0.38 | 0.4584 | 1.08 | 0.7499 | 0.1621 |
| Onset <18 months & | 16 | 10 | −2.4 | 0.7072 | 19 | −0.63 | 0.5381 | 1.77 | 0.8907 | 0.0588 |
| pre-baseline | 24 | 9 | −2.86 | 0.7937 | 18 | −1.05 | 0.5939 | 1.81 | 0.9973 | 0.0823 |
| slope >=1 | | | | | | | | | | |

Pridopidine demonstrates beneficial effects in different subdomains of the ALSFRS-R respiratory scale. Dyspnea is the medical term for shortness of breath and is described as an intense tightening in the chest, breathlessness, or a feeling of suffocation. Pridopidine demonstrates a beneficial effect on dyspnea that is stronger and more statistically significant in subjects who are early with <18 months from symptom onset, faster progressors with a pre-baseline slope ≥1 and with a definite or probable ALS diagnosis (See table 3a).

TABLE 3a
Pridopidine shows less decline vs placebo in ALSFRS-R Respiratory-dyspnea in_ALS subjects.
Change from baseline to week 8, 16 and 24 in different groups.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 152 | −0.12 | 0.0699 | 110 | −0.15 | 0.082 | −0.03 | 0.1083 | 0.805 |
| | 16 | 145 | −0.28 | 0.0792 | 100 | −0.14 | 0.0942 | 0.14 | 0.1239 | 0.2574 |
| | 24 | 57 | −0.47 | 0.1105 | 97 | −0.21 | 0.0971 | 0.26 | 0.1477 | 0.0869 |
| FAS + Onset | 8 | 54 | −0.25 | 0.1026 | 48 | −0.07 | 0.1088 | 0.18 | 0.1516 | 0.2361 |
| <18 Months | 16 | 49 | −0.52 | 0.1229 | 46 | −0.09 | 0.1267 | 0.43 | 0.179 | 0.0166 |
| | 24 | 21 | −0.77 | 0.2006 | 44 | −0.16 | 0.1546 | 0.61 | 0.2547 | 0.0179 |
| FAS + Alsfrs-R | 8 | 19 | −0.47 | 0.217 | 26 | −0.11 | 0.1825 | 0.36 | 0.29 | 0.2299 |
| Pre baseline | 16 | 16 | −0.91 | 0.2349 | 24 | −0.15 | 0.1928 | 0.76 | 0.312 | 0.0189 |
| slope >=1 | 24 | 8 | −1.01 | 0.3281 | 23 | −0.18 | 0.2111 | 0.83 | 0.3985 | 0.0427 |
| FAS + <18 and | 8 | 14 | −0.6 | 0.2895 | 20 | −0.17 | 0.2408 | 0.43 | 0.3873 | 0.2715 |
| slope >=1 | 16 | 11 | −1.27 | 0.3162 | 19 | −0.23 | 0.2497 | 1.04 | 0.4147 | 0.0187 |
| | 24 | 6 | −1.33 | 0.4489 | 18 | −0.12 | 0.2979 | 1.21 | 0.5556 | 0.0393 |
| Definite | 8 | 59 | −0.06 | 0.1296 | 45 | −0.22 | 0.1494 | −0.16 | 0.2 | 0.4231 |
| | 16 | 56 | −0.34 | 0.15 | 39 | −0.27 | 0.1777 | 0.07 | 0.2366 | 0.7697 |
| | 24 | 28 | −0.48 | 0.189 | 37 | −0.25 | 0.181 | 0.23 | 0.2666 | 0.3783 |
| Probable | 8 | 40 | −0.26 | 0.1348 | 37 | −0.01 | 0.1386 | 0.25 | 0.1958 | 0.2176 |
| | 16 | 38 | −0.47 | 0.1544 | 36 | 0.07 | 0.1576 | 0.54 | 0.2235 | 0.0199 |
| | 24 | 14 | −0.36 | 0.1952 | 34 | −0.05 | 0.1351 | 0.31 | 0.2427 | 0.2142 |
| Definite + Probable | 8 | 99 | −0.12 | 0.0934 | 82 | −0.14 | 0.1021 | −0.02 | 0.1392 | 0.8913 |
| | 16 | 94 | −0.37 | 0.1073 | 75 | −0.13 | 0.1186 | 0.24 | 0.1612 | 0.1305 |
| | 24 | 42 | −0.44 | 0.1324 | 71 | −0.17 | 0.1133 | 0.27 | 0.1756 | 0.1347 |
| Definite + Probable & | 8 | 33 | −0.27 | 0.1274 | 36 | −0.08 | 0.1216 | 0.19 | 0.1771 | 0.2809 |
| <18 m | 16 | 29 | −0.68 | 0.1723 | 35 | −0.09 | 0.1578 | 0.59 | 0.2356 | 0.0147 |
| | 24 | 14 | −0.75 | 0.2529 | 33 | −0.14 | 0.1766 | 0.61 | 0.3089 | 0.0531 |
| Definite + Probable & | 8 | 17 | −0.5 | 0.2161 | 24 | −0.13 | 0.1791 | 0.37 | 0.2867 | 0.2113 |
| Pre baseline | 16 | 14 | −0.94 | 0.2511 | 22 | −0.17 | 0.2009 | 0.77 | 0.3308 | 0.0272 |
| slope >=1 | 24 | 7 | −1.22 | 0.3479 | 21 | −0.15 | 0.216 | 1.07 | 0.4222 | 0.0162 |
| Definite + Probable & | 8 | 12 | −0.62 | 0.2921 | 19 | −0.18 | 0.2315 | 0.44 | 0.3805 | 0.2613 |
| <18 m & baseline | 16 | 9 | −1.3 | 0.3477 | 18 | −0.25 | 0.2569 | 1.05 | 0.441 | 0.026 |
| slope >=1 | 24 | 5 | −1.53 | 0.4633 | 17 | −0.12 | 0.2799 | 1.41 | 0.5582 | 0.0191 |

Orthopnea is the sensation of breathlessness in the recumbent position which is alleviated by sitting or standing. Pridopidine demonstrates a trend towards improvement in orthopnea (Table 3b).

TABLE 3b
Pridopidine shows less decline vs placebo in in ALSFRS-R Respiratory-Orthopnea in ALS subjects.
Change from baseline to week 8, 16 and 24 in different groups.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 152 | −0.14 | 0.0646 | 110 | −0.05 | 0.0758 | 0.09 | 0.1001 | 0.378 |
| | 16 | 145 | −0.31 | 0.0875 | 100 | −0.26 | 0.1037 | 0.05 | 0.1365 | 0.7086 |
| | 24 | 57 | −0.43 | 0.1196 | 97 | −0.36 | 0.1109 | 0.07 | 0.1637 | 0.6765 |
| FAS + Symptom | 8 | 54 | −0.19 | 0.1117 | 48 | −0.1 | 0.1185 | 0.09 | 0.1651 | 0.5746 |
| Onset <18 Months | 16 | 49 | −0.42 | 0.1767 | 46 | −0.4 | 0.1838 | 0.02 | 0.2581 | 0.9346 |
| FAS + <18 & re | 8 | 14 | −0.53 | 0.2953 | 20 | −0.12 | 0.2456 | 0.41 | 0.3954 | 0.3055 |
| baseline slope >=1 | 16 | 11 | −0.43 | 0.3644 | 19 | −0.33 | 0.2971 | 0.1 | 0.4794 | 0.839 |
| Definite | 8 | 59 | −0.17 | 0.1007 | 45 | −0.11 | 0.1161 | 0.06 | 0.1553 | 0.6925 |
| | 16 | 56 | −0.33 | 0.1126 | 39 | −0.31 | 0.1323 | 0.02 | 0.1765 | 0.9263 |
| | 24 | 28 | −0.53 | 0.1652 | 37 | −0.52 | 0.1574 | 0.01 | 0.2327 | 0.982 |
| Probable | 8 | 40 | −0.34 | 0.1582 | 37 | 0.05 | 0.1616 | 0.39 | 0.2295 | 0.0959 |
| | 16 | 38 | −0.63 | 0.1842 | 36 | −0.06 | 0.1876 | 0.57 | 0.2665 | 0.0357 |
| | 24 | 14 | −0.8 | 0.2667 | 34 | 0.04 | 0.2206 | 0.84 | 0.3498 | 0.0182 |
| Definite + Probable | 8 | 99 | −0.24 | 0.088 | 82 | −0.05 | 0.0961 | 0.19 | 0.1311 | 0.1449 |
| | 16 | 94 | −0.45 | 0.101 | 75 | −0.2 | 0.111 | 0.25 | 0.1512 | 0.1056 |
| | 24 | 42 | −0.59 | 0.146 | 71 | −0.28 | 0.1308 | 0.31 | 0.1973 | 0.1194 |
| Definite + Probable & | 8 | 33 | −0.44 | 0.1372 | 36 | −0.09 | 0.131 | 0.35 | 0.1907 | 0.0744 |
| Onset <18 months | 16 | 29 | −0.63 | 0.2043 | 35 | −0.24 | 0.1895 | 0.39 | 0.2806 | 0.1729 |
| | 24 | 14 | −0.54 | 0.2391 | 33 | −0.45 | 0.1861 | 0.09 | 0.3036 | 0.7653 |
| Definite + Probable & | 8 | 12 | −0.72 | 0.311 | 19 | −0.1 | 0.2465 | 0.62 | 0.4049 | 0.1366 |
| Onset <18 months & | 16 | 9 | −0.56 | 0.4085 | 18 | −0.33 | 0.3149 | 0.23 | 0.5218 | 0.6553 |
| pre-baseline | 24 | 5 | −0.84 | 0.4253 | 17 | −0.67 | 0.2689 | 0.17 | 0.5186 | 0.7535 |
| slope >=1 | | | | | | | | | | |

Respiratory insufficiency is broadly defined as the impairment of gas exchange between air and circulating blood. Pridopidine has a beneficial effect on the decline seen in ALS subjects. The effect is most notable in definite+probable subjects <18 months from symptom onset and with pre-baseline slope ≥1 (Table 3c).

TABLE 3c
Pridopidine shows less decline vs placebo in Respiratory-Insufficiency in ALS subjects.
Change from baseline to week 8, 16 and 24 in different groups.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 152 | −0.09 | 0.0362 | 110 | −0.08 | 0.0424 | 0.01 | 0.056 | 0.8552 |
| | 16 | 145 | −0.17 | 0.0479 | 100 | −0.11 | 0.0567 | 0.06 | 0.0747 | 0.4262 |
| | 24 | 57 | −0.29 | 0.0699 | 97 | −0.19 | 0.0654 | 0.1 | 0.0962 | 0.3102 |
| Probable | 8 | 40 | −0.19 | 0.0881 | 37 | −0.15 | 0.0901 | 0.04 | 0.1279 | 0.7648 |
| | 16 | 38 | −0.26 | 0.1043 | 36 | −0.19 | 0.1061 | 0.07 | 0.1507 | 0.6517 |
| | 24 | 14 | −0.32 | 0.1268 | 34 | −0.25 | 0.1168 | 0.07 | 0.1745 | 0.6893 |
| Definite + Probable | 8 | 99 | −0.12 | 0.0475 | 82 | −0.08 | 0.0519 | 0.04 | 0.0708 | 0.5437 |
| | 16 | 94 | −0.2 | 0.0631 | 75 | −0.1 | 0.0695 | 0.1 | 0.0946 | 0.284 |
| | 24 | 42 | −0.39 | 0.0885 | 71 | −0.18 | 0.0835 | 0.21 | 0.1225 | 0.0907 |
| Definite + Probable & | 8 | 33 | −0.09 | 0.0734 | 36 | −0.08 | 0.0701 | 0.01 | 0.102 | 0.967 |
| Onset <18 months | 16 | 29 | −0.12 | 0.0878 | 35 | −0.03 | 0.0815 | 0.09 | 0.1207 | 0.4607 |
| | 24 | 14 | −0.16 | 0.0997 | 33 | −0.14 | 0.0716 | 0.02 | 0.1231 | 0.8319 |

Pridopidine demonstrates a significant, mitigating effect vs. placebo on ALSFRS-R respiratory score in definite+probable subjects analyzed with the MMRM model (change vs. baseline 0.73, p=0.02). The effect is larger in subjects <18 months from symptom onset (change vs. placebo 1.2, p=0.03) (FIG. 28).

Time-course analysis demonstrates that pridopidine shows a trend for mitigating the decline in ALSFRS-R respiratory score from week 8 in definite+probable and definite+probable subjects <18 months from symptom onset, and from 16 weeks in FAS with pre-baseline slope ³ 0.75 and definite ALS subjects <18 months from symptom onset (FIG. 28).

The effect of pridopidine on respiratory parameters SVC % and FVC % was also evaluated in the study. Pridopidine shows a trend towards improvement in SVC % (Table 4) and FVC % (Table 5), with the changes most notable in definite+probable subjects <18 months from symptom onset.

TABLE 4
Pridopidine shows less decline vs placebo SVC % in ALS subjects.
Change from baseline to week 8, 16 and 24 in different groups.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS + Symptom | 24 | 40 | −11.43 | 1.9784 | 32 | −8.31 | 2.192 | 3.12 | 2.9648 | 0.2949 |
| Onset <18 Months | | | | | | | | | | |
| FAS + ALSFRS-R | 8 | 16 | −6.44 | 2.8105 | 21 | −3.42 | 2.4456 | 3.02 | 3.8169 | 0.4333 |
| Pre baseline | 16 | 12 | −11.15 | 3.6929 | 18 | −9.81 | 3.1144 | 1.34 | 4.9756 | 0.7889 |
| slope >=1 | 24 | 10 | −16.69 | 4.8688 | 18 | −14.2 | 3.9548 | 2.49 | 6.532 | 0.7053 |
| FAS + <18 & re | 8 | 13 | −6.14 | 3.5109 | 17 | −4.32 | 3.1372 | 1.82 | 4.9257 | 0.7154 |
| baseline slope >=1 | 16 | 11 | −11.66 | 4.5304 | 14 | −10.71 | 4.0363 | 0.95 | 6.2989 | 0.8822 |
| | 24 | 7 | −17.66 | 6.5829 | 13 | −15.05 | 5.2566 | 2.61 | 8.9108 | 0.7718 |
| Definite | 8 | 49 | −4.96 | 1.1932 | 32 | −1.28 | 1.4866 | 3.68 | 1.9271 | 0.0592 |
| | 16 | 44 | −8.1 | 1.7408 | 24 | −6.4 | 2.2641 | 1.7 | 2.8979 | 0.5608 |
| | 24 | 37 | −10.89 | 1.836 | 29 | −8.9 | 2.0867 | 1.99 | 2.8161 | 0.483 |
| Definite + Onset | 8 | 15 | −4.03 | 2.4633 | 16 | −1.61 | 2.4988 | 2.42 | 3.6595 | 0.5144 |
| <18 Months | 16 | 15 | −10.44 | 2.623 | 11 | −8.93 | 2.8885 | 1.51 | 4.0776 | 0.7142 |
| | 24 | 12 | −15.15 | 3.3351 | 12 | −7.58 | 3.2988 | 7.57 | 4.8239 | 0.1296 |
| Definite + Probable & | 8 | 29 | −3.75 | 1.8395 | 32 | −3.57 | 1.7693 | 0.18 | 2.5657 | 0.944 |
| Onset <18 months | 16 | 27 | −8.47 | 2.347 | 26 | −7.68 | 2.3434 | 0.79 | 3.3464 | 0.8159 |
| | 24 | 22 | −14.21 | 2.7679 | 26 | −8.34 | 2.6197 | 5.87 | 3.8412 | 0.1326 |
| Definite + Probable & | 8 | 13 | −10.68 | 2.5045 | 19 | −3.22 | 2.0252 | 7.46 | 3.3393 | 0.0335 |
| Pre baseline | 16 | 10 | −13.02 | 4.1648 | 16 | −10.3 | 3.3635 | 2.72 | 5.5376 | 0.6272 |
| slope >=1 | 24 | 8 | −19.74 | 5.7539 | 16 | −14.9 | 4.3532 | 4.84 | 7.6577 | 0.5323 |
| Definite + Probable & | 8 | 11 | −10.65 | 2.7587 | 16 | −4.06 | 2.3194 | 6.59 | 3.7377 | 0.094 |
| Onset <18 months & | 16 | 9 | −13.9 | 4.8801 | 13 | −10.91 | 4.0736 | 2.99 | 6.5584 | 0.653 |
| pre-baseline | 24 | 5 | −20.22 | 7.9625 | 12 | −15.66 | 5.32 | 4.56 | 10.4079 | 0.6665 |
| slope >=1 | | | | | | | | | | |

TABLE 5
Pridopidine shows less decline vs placebo FVC % in ALS subjects.
Change from baseline to week 8, 16 and 24 in different groups.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 24 | 23 | −18.46 | 3.4469 | 28 | −13.83 | 3.2805 | 4.63 | 4.8103 | 0.3388 |
| FAS + Symptom | 8 | 22 | −8.45 | 2.232 | 24 | −4.22 | 2.0374 | 4.23 | 3.2142 | 0.1952 |
| Onset <18 Months | 16 | 21 | −16.6 | 3.1119 | 24 | −7.26 | 2.8338 | 9.34 | 4.4113 | 0.0399 |
| | 24 | 9 | −23.45 | 6.2059 | 15 | −11.32 | 4.9672 | 12.13 | 7.9441 | 0.1337 |
| FAS + ALSFRS-R | 24 | 4 | −32.75 | 12.2779 | 7 | −24.14 | 8.5977 | 8.61 | 15.358 | 0.5835 |
| Pre baseline | | | | | | | | | | |
| slope >=1 | | | | | | | | | | |
| FAS + <18 & re | 8 | 4 | −23.26 | 9.0839 | 11 | −4.3 | 4.5995 | 18.96 | 11.1448 | 0.1196 |
| baseline slope >=1 | 16 | 4 | −36 | 10.0879 | 13 | −17.18 | 4.9942 | 18.82 | 11.9963 | 0.1478 |
| | 24 | 3 | −44.67 | 13.5587 | 6 | −18.43 | 8.3767 | 26.24 | 16.5799 | 0.1447 |
| Definite | 24 | 10 | −25.58 | 6.6729 | 8 | −19.87 | 7.4904 | 5.71 | 10.1913 | 0.5787 |
| Probable | 8 | 15 | −7.33 | 3.1888 | 15 | −6.03 | 3.391 | 1.3 | 4.9269 | 0.7941 |
| | 16 | 17 | −10.62 | 4.1609 | 15 | −8.68 | 4.4639 | 1.94 | 6.2648 | 0.7593 |
| | 24 | 9 | −12.46 | 4.9743 | 10 | −9.76 | 5.1732 | 2.7 | 7.295 | 0.7144 |
| Definite + Probable | 24 | 19 | −19.98 | 3.8915 | 18 | −13.24 | 4.0807 | 6.74 | 5.7275 | 0.2429 |
| Definite + Probable & | 8 | 14 | −11.07 | 3.0959 | 19 | −6.04 | 2.5761 | 5.03 | 4.2304 | 0.2443 |
| Onset <18 months | 16 | 14 | −23.77 | 3.8935 | 20 | −11.03 | 3.2049 | 12.74 | 5.2481 | 0.0214 |
| | 24 | 7 | −32.42 | 7.542 | 11 | −13.37 | 6.0374 | 19.05 | 9.7608 | 0.0603 |
| Definite + Probable & | 8 | 7 | −11.64 | 6.4108 | 12 | −10.02 | 4.0641 | 1.62 | 8.2282 | 0.8469 |
| Pre baseline | 16 | 6 | −21.64 | 7.896 | 13 | −18.91 | 5.1707 | 2.73 | 10.1672 | 0.7923 |
| slope >=1 | 24 | 4 | −33.85 | 12.6537 | 6 | −21.34 | 9.2303 | 12.51 | 16.5016 | 0.4609 |
| Definite + Probable & | 8 | 4 | −23.06 | 9.0984 | 11 | −4.1 | 4.5711 | 18.96 | 11.1448 | 0.1196 |
| Onset <18 months & | 16 | 3 | −44.46 | 13.5754 | 6 | −18.23 | 8.3434 | 26.23 | 16.5799 | 0.1447 |
| pre-baseline | 24 | 4 | −35.8 | 10.1008 | 13 | −16.98 | 4.9703 | 18.82 | 11.9963 | 0.1478 |
| slope >=1 | | | | | | | | | | |

The effect of pridopidine on bulbar functions was evaluated using the ALSFRS-R bulbar score as well as by the CNS-BFS.

Pridopidine demonstrates a trend towards mitigating the decline in the ALSFRS-R Bulbar Score (FIG. 30 and Table 6). This effect is larger in definite subjects <18 months from symptom onset (FIGS. 30 and 31). Similarly, the mitigating effect on bulbar functions are larger in definite +probable ALS subjects (FIG. 32). Tables 7A-C demonstrate the effect of pridopidine on sections of the bulbar scale speech, salivation, and swallowing.

TABLE 6
Pridopidine shows less decline vs placebo in ASLFRS-R Bulbar in ALS subjects.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 155 | −0.52 | 0.0788 | 112 | −0.47 | 0.0925 | 0.05 | 0.122 | 0.7027 |
| | 16 | 148 | −1.01 | 0.1101 | 104 | −0.89 | 0.1303 | 0.12 | 0.1716 | 0.4916 |
| | 24 | 143 | −1.4 | 0.1402 | 99 | −1.28 | 0.1668 | 0.12 | 0.2192 | 0.6022 |
| FAS + Symptom | 8 | 54 | −0.53 | 0.1435 | 49 | −0.58 | 0.1512 | −0.05 | 0.2108 | 0.8304 |
| Onset <18 Months | 16 | 52 | −1.26 | 0.1978 | 47 | −0.75 | 0.2086 | 0.51 | 0.2913 | 0.0818 |
| | 24 | 49 | −1.49 | 0.2529 | 46 | −1.18 | 0.2644 | 0.31 | 0.3702 | 0.396 |
| FAS + ALSFRS-R | 8 | 20 | −0.52 | 0.2513 | 27 | −0.65 | 0.2148 | −0.13 | 0.3341 | 0.7099 |
| Pre baseline | 16 | 17 | −1.9 | 0.4172 | 26 | −1.43 | 0.3463 | 0.47 | 0.5438 | 0.3835 |
| slope >=1 | 24 | 16 | −2.25 | 0.5216 | 24 | −1.83 | 0.4335 | 0.42 | 0.68 | 0.5411 |
| FAS + <18 & re | 8 | 14 | −0.6 | 0.2891 | 20 | −0.59 | 0.2407 | 0.01 | 0.3826 | 0.9763 |
| baseline slope >=1 | 16 | 12 | −2 | 0.4521 | 20 | −1.03 | 0.3593 | 0.97 | 0.582 | 0.1091 |
| | 24 | 11 | −2.25 | 0.5715 | 19 | −1.52 | 0.4588 | 0.73 | 0.7372 | 0.3325 |
| Definite | 8 | 61 | −0.63 | 0.1182 | 46 | −0.43 | 0.1361 | 0.2 | 0.1825 | 0.2845 |
| | 16 | 56 | −1.35 | 0.192 | 42 | −1.19 | 0.223 | 0.16 | 0.2996 | 0.5973 |
| | 24 | 56 | −2.02 | 0.2443 | 38 | −1.72 | 0.2877 | 0.3 | 0.3841 | 0.4396 |
| Definite + Onset | 8 | 18 | −0.77 | 0.238 | 19 | −0.73 | 0.2292 | 0.04 | 0.3456 | 0.9219 |
| <18 Months | 16 | 16 | −1.9 | 0.3495 | 20 | −0.72 | 0.3158 | 1.18 | 0.4923 | 0.0228 |
| | 24 | 16 | −2.35 | 0.4808 | 19 | −1.53 | 0.4352 | 0.82 | 0.6718 | 0.2321 |
| Probable | 8 | 40 | −0.78 | 0.1842 | 37 | −0.54 | 0.187 | 0.24 | 0.2652 | 0.3654 |
| | 16 | 40 | −1.23 | 0.2118 | 37 | −0.8 | 0.2158 | 0.43 | 0.3049 | 0.1582 |
| | 24 | 36 | −1.55 | 0.2635 | 35 | −1.01 | 0.2691 | 0.54 | 0.3799 | 0.1615 |
| Definite + Probable | 8 | 101 | −0.67 | 0.103 | 83 | −0.5 | 0.1125 | 0.17 | 0.1534 | 0.2668 |
| | 16 | 96 | −1.29 | 0.1426 | 79 | −1.03 | 0.1563 | 0.26 | 0.213 | 0.2144 |
| | 24 | 92 | −1.8 | 0.1811 | 73 | −1.45 | 0.201 | 0.35 | 0.2724 | 0.1933 |
| Definite + Probable & | 8 | 33 | −0.82 | 0.2012 | 36 | −0.61 | 0.1912 | 0.21 | 0.2794 | 0.4558 |
| Onset <18 months | 16 | 31 | −1.84 | 0.2732 | 36 | −0.92 | 0.2573 | 0.92 | 0.3787 | 0.0174 |
| | 24 | 29 | −2.3 | 0.3497 | 35 | −1.38 | 0.3246 | 0.92 | 0.4812 | 0.0585 |
| Definite + Probable & | 16 | 15 | −1.93 | 0.4613 | 24 | −1.49 | 0.3683 | 0.44 | 0.5943 | 0.4717 |
| Pre baseline | 24 | 14 | −2.24 | 0.5628 | 22 | −1.83 | 0.4511 | 0.41 | 0.729 | 0.573 |
| slope >=1 | | | | | | | | | | |
| Definite + Probable & | 8 | 12 | −0.62 | 0.3203 | 19 | −0.63 | 0.2514 | −0.01 | 0.4126 | 0.9873 |
| Onset <18 months & | 16 | 10 | −2.28 | 0.5096 | 19 | −1.1 | 0.3814 | 1.18 | 0.639 | 0.0775 |
| pre-baseline | 24 | 9 | −2.44 | 0.6454 | 18 | −1.63 | 0.4861 | 0.81 | 0.8127 | 0.3318 |
| slope >=1 | | | | | | | | | | |

TABLE 7a
pridopidine shows less decline vs placebo in ASLFRS-R Bulbar-speech in ALS subjects.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 152 | −0.2 | 0 | 110 | −0.19 | 0.046 | 0.01 | 0.0607 | 0.8609 |
| | 16 | 145 | −0.39 | 0.1 | 100 | −0.29 | 0.0593 | 0.1 | 0.078 | 0.2097 |
| | 24 | 57 | −0.51 | 0.1 | 97 | −0.38 | 0.0681 | 0.13 | 0.1019 | 0.2092 |
| FAS + Symptom | 16 | 49 | −0.44 | 0.1 | 46 | −0.25 | 0.0931 | 0.19 | 0.1309 | 0.159 |
| Onset <18 Months | 24 | 21 | −0.48 | 0.1 | 44 | −0.37 | 0.1101 | 0.11 | 0.1782 | 0.5263 |
| FAS + <18 & re | 16 | 11 | −0.55 | 0.2 | 19 | −0.38 | 0.1494 | 0.17 | 0.2484 | 0.5036 |
| baseline slope >=1 | 24 | 6 | −1.06 | 0.3 | 18 | −0.49 | 0.2092 | 0.57 | 0.3611 | 0.1229 |
| Definite | 8 | 59 | −0.29 | 0.1 | 45 | −0.22 | 0.0809 | 0.07 | 0.1082 | 0.52 |
| | 16 | 56 | −0.51 | 0.1 | 39 | −0.38 | 0.1094 | 0.13 | 0.1459 | 0.38 |
| | 24 | 28 | −0.66 | 0.1 | 37 | −0.43 | 0.1109 | 0.23 | 0.1588 | 0.1567 |
| Probable | 8 | 40 | −0.25 | 0.1 | 37 | −0.13 | 0.0754 | 0.12 | 0.1067 | 0.2568 |
| | 16 | 38 | −0.54 | 0.1 | 36 | −0.22 | 0.0907 | 0.32 | 0.1287 | 0.0164 |
| | 24 | 14 | −0.87 | 0.2 | 34 | −0.41 | 0.1169 | 0.46 | 0.2137 | 0.0351 |
| Definite + Probable | 8 | 99 | −0.27 | 0.1 | 82 | −0.19 | 0.0559 | 0.08 | 0.0762 | 0.3523 |
| | 16 | 94 | −0.51 | 0.1 | 75 | −0.33 | 0.0722 | 0.18 | 0.0982 | 0.0623 |
| | 24 | 42 | −0.67 | 0.1 | 71 | −0.45 | 0.0823 | 0.22 | 0.1256 | 0.078 |
| Definite + Probable & | 16 | 29 | −0.69 | 0.1 | 35 | −0.31 | 0.1096 | 0.38 | 0.1627 | 0.0229 |
| Onset <18 months | 24 | 14 | −0.81 | 0.2 | 33 | −0.49 | 0.1362 | 0.32 | 0.2305 | 0.1711 |
| Definite + Probable & | 16 | 14 | −0.52 | 0.2 | 22 | −0.47 | 0.1307 | 0.05 | 0.217 | 0.8312 |
| Pre baseline slope >=1 | 24 | 7 | −0.83 | 0.2 | 21 | −0.61 | 0.1782 | 0.22 | 0.3092 | 0.4896 |
| Definite + Probable & | 16 | 9 | −0.67 | 0.2 | 18 | −0.39 | 0.1594 | 0.28 | 0.2742 | 0.3166 |
| Onset <18 months & | 24 | 5 | −1.08 | 0.3 | 17 | −0.51 | 0.2289 | 0.57 | 0.4056 | 0.1714 |
| pre-baseline slope >=1 | | | | | | | | | | |

TABLE 7b
Pridopidine shows less decline vs placebo in ASLFRS-R Bulbar-salivation in ALS subjects.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS + Symptom | 8 | 54 | −0.28 | 0.0807 | 48 | −0.21 | 0.0857 | 0.07 | 0.1193 | 0.5706 |
| Onset <18 Months | 16 | 49 | −0.44 | 0.1342 | 46 | −0.25 | 0.1396 | 0.19 | 0.196 | 0.3338 |
| | 24 | 21 | −0.41 | 0.2137 | 44 | −0.39 | 0.1727 | 0.02 | 0.2764 | 0.9645 |
| FAS + ALSFRS-R | 16 | 16 | −0.56 | 0.2957 | 24 | −0.48 | 0.2418 | 0.08 | 0.3867 | 0.8371 |
| Pre baseline slope >=1 | 24 | 8 | −0.53 | 0.4413 | 23 | −0.43 | 0.3207 | 0.1 | 0.5493 | 0.8472 |
| FAS + <18 & re | 8 | 14 | −0.29 | 0.1883 | 20 | −0.15 | 0.1567 | 0.14 | 0.2519 | 0.6027 |
| baseline slope >=1 | 16 | 11 | −0.7 | 0.3465 | 19 | −0.27 | 0.2691 | 0.43 | 0.4435 | 0.3461 |
| | 24 | 6 | −0.91 | 0.5508 | 18 | −0.15 | 0.3781 | 0.76 | 0.6796 | 0.2742 |
| Definite | 8 | 59 | −0.19 | 0.0787 | 45 | −0.15 | 0.0908 | 0.04 | 0.1215 | 0.7424 |
| | 16 | 56 | −0.48 | 0.1165 | 39 | −0.43 | 0.1385 | 0.05 | 0.1845 | 0.7736 |
| | 24 | 28 | −0.7 | 0.1657 | 37 | −0.64 | 0.1707 | 0.06 | 0.2424 | 0.7991 |
| Probable | 8 | 40 | −0.28 | 0.1051 | 37 | −0.18 | 0.1076 | 0.1 | 0.1527 | 0.5366 |
| | 16 | 38 | −0.32 | 0.1313 | 36 | −0.28 | 0.1337 | 0.04 | 0.1898 | 0.8044 |
| | 24 | 14 | −0.31 | 0.2602 | 34 | −0.32 | 0.1852 | −0.01 | 0.3236 | 0.9807 |
| Definite + Probable | 8 | 99 | −0.22 | 0.0633 | 82 | −0.17 | 0.0693 | 0.05 | 0.0945 | 0.5607 |
| | 16 | 94 | −0.41 | 0.0848 | 75 | −0.35 | 0.0939 | 0.06 | 0.1276 | 0.6338 |
| | 24 | 42 | −0.56 | 0.1389 | 71 | −0.47 | 0.1219 | 0.09 | 0.186 | 0.6412 |
| Definite + Probable & | 8 | 33 | −0.36 | 0.1183 | 36 | −0.23 | 0.1129 | 0.13 | 0.1644 | 0.4195 |
| Onset <18 months | 16 | 29 | −0.57 | 0.1988 | 35 | −0.29 | 0.1832 | 0.28 | 0.2724 | 0.3184 |
| | 24 | 14 | −0.57 | 0.3002 | 33 | −0.39 | 0.2264 | 0.18 | 0.3767 | 0.628 |
| Definite + Probable & | 8 | 17 | −0.05 | 0.1893 | 24 | −0.2 | 0.1566 | −0.15 | 0.2508 | 0.5594 |
| Pre baseline slope >=1 | 16 | 14 | −0.56 | 0.3332 | 22 | −0.53 | 0.2647 | 0.03 | 0.4339 | 0.951 |
| | 24 | 7 | −0.62 | 0.4945 | 21 | −0.4 | 0.3524 | 0.22 | 0.6153 | 0.7328 |
| Definite + Probable & | 8 | 12 | −0.26 | 0.2079 | 19 | −0.16 | 0.1647 | 0.1 | 0.2707 | 0.7124 |
| Onset <18 months & | 16 | 9 | −0.76 | 0.3984 | 18 | −0.28 | 0.2874 | 0.48 | 0.4958 | 0.3508 |
| pre-baseline slope >=1 | 24 | 5 | −1.1 | 0.6298 | 17 | −0.15 | 0.405 | 0.95 | 0.7611 | 0.2216 |

TABLE 7c
Pridopidine shows less decline vs placebo in ASLFRS-R Bulbar-swallowing in ALS subjects.
Positive change indicates improvement

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 152 | −0.17 | 0.0398 | 110 | −0.13 | 0.0467 | 0.04 | 0.0617 | 0.4643 |
| | 16 | 145 | −0.29 | 0.052 | 100 | −0.3 | 0.0618 | −0.01 | 0.0813 | 0.9191 |
| | 24 | 57 | −0.47 | 0.0859 | 97 | −0.39 | 0.0795 | 0.08 | 0.1176 | 0.5083 |
| FAS + Symptom | 8 | 54 | −0.13 | 0.0645 | 48 | −0.13 | 0.0684 | 0 | 0.0953 | 0.9551 |
| Onset <18 Months | 16 | 49 | −0.35 | 0.0849 | 46 | −0.22 | 0.0884 | 0.13 | 0.1241 | 0.2967 |
| | 24 | 21 | −0.48 | 0.1357 | 44 | −0.24 | 0.1066 | 0.24 | 0.1736 | 0.171 |
| FAS + ALSFRS-R | 8 | 19 | −0.25 | 0.132 | 26 | −0.23 | 0.1108 | 0.02 | 0.1765 | 0.9087 |
| Pre baseline slope >=1 | 16 | 16 | −0.62 | 0.1981 | 24 | −0.43 | 0.1621 | 0.19 | 0.2603 | 0.4645 |
| | 24 | 8 | −0.76 | 0.337 | 23 | −0.55 | 0.2206 | 0.21 | 0.4077 | 0.6167 |
| FAS + <18 & re | 8 | 14 | −0.24 | 0.1609 | 20 | −0.23 | 0.1339 | 0.01 | 0.215 | 0.9906 |
| baseline slope >=1 | 16 | 11 | −0.58 | 0.2226 | 19 | −0.39 | 0.1744 | 0.19 | 0.2879 | 0.5219 |
| | 24 | 6 | −0.64 | 0.3561 | 18 | −0.48 | 0.2146 | 0.16 | 0.4277 | 0.7153 |
| Definite | 8 | 59 | −0.18 | 0.068 | 45 | −0.06 | 0.0784 | 0.12 | 0.1049 | 0.2253 |
| | 16 | 56 | −0.39 | 0.0945 | 39 | −0.36 | 0.1118 | 0.03 | 0.149 | 0.8786 |
| | 24 | 28 | −0.6 | 0.147 | 37 | −0.53 | 0.1491 | 0.07 | 0.2135 | 0.764 |
| Probable | 8 | 40 | −0.27 | 0.0885 | 37 | −0.26 | 0.0909 | 0.01 | 0.1287 | 0.9513 |
| | 16 | 38 | −0.38 | 0.1072 | 36 | −0.39 | 0.1094 | −0.01 | 0.1551 | 0.9734 |
| | 24 | 14 | −0.64 | 0.1646 | 34 | −0.32 | 0.1154 | 0.32 | 0.2049 | 0.1259 |
| Definite + Probable | 8 | 99 | −0.22 | 0.0545 | 82 | −0.16 | 0.0597 | 0.06 | 0.0813 | 0.45 |
| | 16 | 94 | −0.39 | 0.0701 | 75 | −0.37 | 0.0774 | 0.02 | 0.1053 | 0.8901 |
| | 24 | 42 | −0.57 | 0.1099 | 71 | −0.45 | 0.0993 | 0.12 | 0.1491 | 0.4288 |
| Definite + Probable & | 8 | 33 | −0.24 | 0.093 | 36 | −0.19 | 0.0888 | 0.05 | 0.1292 | 0.6919 |
| Onset <18 months | 16 | 29 | −0.54 | 0.1254 | 35 | −0.32 | 0.116 | 0.22 | 0.1721 | 0.1983 |
| | 24 | 14 | −0.69 | 0.1724 | 33 | −0.29 | 0.127 | 0.4 | 0.2148 | 0.0728 |
| Definite + Probable & | 8 | 17 | −0.27 | 0.1439 | 24 | −0.26 | 0.1194 | 0.01 | 0.1914 | 0.9534 |
| Pre baseline slope >=1 | 16 | 14 | −0.69 | 0.2218 | 22 | −0.47 | 0.1765 | 0.22 | 0.2902 | 0.4469 |
| | 24 | 7 | −0.81 | 0.3844 | 21 | −0.57 | 0.2456 | 0.24 | 0.4662 | 0.6033 |
| Definite + Probable & | 8 | 12 | −0.27 | 0.1761 | 19 | −0.25 | 0.1396 | 0.02 | 0.2292 | 0.9423 |
| Onset <18 months & | 16 | 9 | −0.69 | 0.255 | 18 | −0.42 | 0.1867 | 0.27 | 0.3203 | 0.4016 |
| pre-baseline slope >=1 | 24 | 5 | −0.72 | 0.4131 | 17 | −0.52 | 0.234 | 0.2 | 0.4882 | 0.6754 |

Pridopidine demonstrates a mitigating effect on the decline in bulbar function in ALS subjects. The effect observed in the FAS is driven by subjects with definite+probable ALS <18 months from symptom onset (Table 8).

Pridopidine demonstrates a significant mitigating effect on speech and swallowing as assessed by the CNS-BFS (Table 8a and 8b). The effects are greater and more significant in subjects with earlier onset and more rapid progression.

TABLE 8
Pridopidine shows less decline vs placebo in CNS-BFS bulbar function.
(negative change indicates improvement)

| | | Placebo | | | Pridopidine | | | | | |
|---|---|---|---|---|---|---|---|---|---|---|
| | | | Change | | | Change | | | | |
| | | | from | | | from | | | | |
| | | | baseline | | | baseline | | Pridopidine vs placebo | | |
| | Week | N | (LS means) | SE | N | (LS means) | SE | (LS means) | SE | P Value |
| FAS | 8 | 155 | 1.76 | 0.5594 | 113 | 1.7 | 0.6564 | −0.06 | 0.866 | 0.9381 |
| | 16 | 148 | 4.53 | 0.6972 | 102 | 3.37 | 0.8303 | −1.16 | 1.0898 | 0.2875 |
| | 24 | 141 | 7.85 | 0.8796 | 100 | 6.85 | 1.0436 | −1 | 1.3737 | 0.4669 |
| FAS + Symptom | 8 | 54 | 1.31 | 1.0096 | 49 | 1.12 | 1.0656 | −0.19 | 1.4864 | 0.9016 |
| Onset <18 Months | 16 | 52 | 4.96 | 1.2292 | 46 | 3.1 | 1.3036 | −1.86 | 1.8143 | 0.3071 |
| | 24 | 48 | 8.2 | 1.7053 | 46 | 6.85 | 1.7629 | −1.35 | 2.4831 | 0.5883 |
| FAS + <18 & pre | 8 | 14 | 1.85 | 2.0153 | 20 | 2.84 | 1.6719 | 0.99 | 2.6926 | 0.716 |
| baseline slope >=1 | 16 | 13 | 10.69 | 3.6184 | 19 | 8.19 | 2.9962 | −2.5 | 4.7503 | 0.6034 |
| | 24 | 11 | 11.17 | 3.6401 | 19 | 8.55 | 2.8758 | −2.62 | 4.6959 | 0.5811 |
| Definite | 8 | | | | | | | | | |
| | 16 | 56 | 7.67 | 1.3088 | 41 | 7 | 1.5274 | −0.67 | 2.0451 | 0.7439 |
| | 24 | 55 | 11.18 | 1.4321 | 39 | 10.47 | 1.693 | −0.71 | 2.2583 | 0.7534 |
| Probable | 8 | 40 | 2.76 | 1.2133 | 37 | −0.29 | 1.2466 | −3.05 | 1.764 | 0.088 |
| | 16 | 40 | 4.41 | 1.1849 | 37 | 0.21 | 1.2173 | −4.2 | 1.7235 | 0.0174 |
| | 24 | 35 | 10.18 | 2.0386 | 35 | 4.71 | 2.0599 | −5.47 | 2.9274 | 0.0662 |
| Definite + Probable | 8 | 100 | 1.99 | 0.7091 | 85 | 2.28 | 0.7702 | 0.29 | 1.0532 | 0.7827 |
| | 16 | 96 | 5.91 | 0.9327 | 78 | 4.38 | 1.026 | −1.53 | 1.3949 | 0.2747 |
| | 24 | 90 | 10.38 | 1.1639 | 74 | 8.16 | 1.2828 | −2.22 | 1.7454 | 0.2061 |
| Definite + Probable & | 8 | 33 | 1.42 | 1.3882 | 36 | 0.78 | 1.327 | −0.64 | 1.9322 | 0.7413 |
| Onset <18 months | 16 | 31 | 6.2 | 1.8077 | 35 | 4.03 | 1.7177 | −2.17 | 2.5089 | 0.3894 |
| | 24 | 28 | 12.31 | 2.5816 | 35 | 8.24 | 2.3568 | −4.07 | 3.526 | 0.2528 |
| Definite + Probable & | 8 | 12 | 1.27 | 2.2233 | 19 | 3.08 | 1.7548 | 1.81 | 2.8929 | 0.5384 |
| Onset <18 months & | 16 | 11 | 11.25 | 3.9437 | 18 | 8.84 | 3.1027 | −2.41 | 5.0607 | 0.6383 |
| pre-baseline slope >=1 | 24 | 9 | 13.18 | 4.2148 | 18 | 8.9 | 3.1402 | −4.28 | 5.3039 | 0.4277 |

TABLE 8a
Pridopidine shows less decline vs placebo in CNS-BFS Speech in ALS subjects.
Negative change indicates improvement

| | | Placebo | | | Pridopidine | | | | | |
|---|---|---|---|---|---|---|---|---|---|---|
| | | | Change | | | Change | | | | |
| | | | from | | | from | | | | |
| Group at | | | baseline | | | baseline | | Pridopidine vs placebo | | |
| 24 weeks | Week | N | (LS means) | SE | N | (LS means) | SE | (LS means) | SE | P Value |
| FAS and | <18 and slope >=1 | 6 | 8.95 | 1.8463 | 18 | 4.03 | 1.4214 | −4.92 | 2.3556 | 0.0465 |
| | <24 and slope >=1 | 6 | 9.19 | 1.6968 | 22 | 4.37 | 1.2237 | −4.82 | 2.1216 | 0.0296 |
| | <18 and slope >=0.9 | 7 | 7.73 | 1.8417 | 23 | 3.95 | 1.211 | −3.78 | 2.2429 | 0.1008 |
| | <24 and slope >=0.9 | 8 | 7.46 | 1.5842 | 27 | 4.21 | 1.0822 | −3.25 | 1.9332 | 0.1001 |
| | Alsfrs-R Pre bl slp >=1 | 8 | 7.04 | 1.7724 | 23 | 5.01 | 1.3358 | −2.03 | 2.2315 | 0.3676 |
| | Probable | 13 | 4.56 | 1.2239 | 34 | 2.69 | 0.8483 | −1.87 | 1.5135 | 0.2216 |
| | <24 and Svc >60% | 25 | 4.61 | 0.8494 | 56 | 3.11 | 0.6503 | −1.5 | 1.075 | 0.1647 |
| | Onset <24 Months | 30 | 4.83 | 0.7676 | 67 | 3.35 | 0.6102 | −1.48 | 0.9867 | 0.1346 |
| | Onset <18 Months | 20 | 4.09 | 0.9363 | 44 | 2.89 | 0.7962 | −1.2 | 1.2382 | 0.3368 |
| | Definite and Probable | 41 | 4.76 | 0.6899 | 71 | 3.88 | 0.5872 | −0.88 | 0.9135 | 0.3326 |
| | Alsfrs-R Pre bl slp >=0.9 | 11 | 5.86 | 1.5023 | 28 | 5 | 1.141 | −0.86 | 1.8983 | 0.6523 |
| | All Patients | 56 | 3.75 | 0.5537 | 97 | 3.23 | 0.4758 | −0.52 | 0.7336 | 0.4834 |
| | Svc >60% | 46 | 3.49 | 0.6068 | 84 | 3.11 | 0.5022 | −0.38 | 0.79 | 0.6323 |
| | Definite | 28 | 4.84 | 0.8804 | 37 | 4.74 | 0.841 | −0.1 | 1.244 | 0.9356 |
| Definite | <18 and slope >=1 | 5 | 10.46 | 1.9652 | 17 | 4.24 | 1.4438 | −6.22 | 2.4584 | 0.0187 |
| and | <18 and slope >=0.9 | 5 | 10.35 | 2.2151 | 20 | 4.28 | 1.3696 | −6.07 | 2.6551 | 0.0307 |
| probable | <24 and slope >=1 | 5 | 10.63 | 1.7615 | 21 | 4.61 | 1.2214 | −6.02 | 2.1704 | 0.0094 |
| and | <24 and slope >=0.9 | 6 | 9.05 | 1.8412 | 24 | 4.71 | 1.1885 | −4.34 | 2.2161 | 0.0585 |
| | Alsfrs-R Pre bl slp >=1 | 7 | 8.36 | 1.6764 | 21 | 4.47 | 1.2382 | −3.89 | 2.1058 | 0.0733 |
| | Alsfrs-R Pre bl slp >=0.9 | 9 | 7.24 | 1.6231 | 24 | 4.7 | 1.1803 | −2.54 | 2.0405 | 0.2207 |
| | Onset <18 Months | 13 | 5.83 | 1.3208 | 33 | 4.06 | 1.0616 | −1.77 | 1.7017 | 0.3021 |
| | Onset <24 Months | 21 | 5.79 | 1.0313 | 49 | 4.32 | 0.8112 | −1.47 | 1.3206 | 0.2679 |
| | <24 and Svc >60% | 16 | 5.69 | 1.2123 | 40 | 4.22 | 0.9073 | −1.47 | 1.5221 | 0.3378 |
| | Definite + Probable | 41 | 4.76 | 0.6899 | 71 | 3.88 | 0.5872 | −0.88 | 0.9135 | 0.3326 |
| | Svc >60% | 33 | 4.38 | 0.774 | 61 | 3.8 | 0.6128 | −0.58 | 0.9954 | 0.5579 |

TABLE 8b
pridopidine shows less decline vs placebo in CNS-BFS Swallowing in ALS subjects.
negative change indicates improvement.

| | | Placebo | | | Pridopidine | | | | | |
|---|---|---|---|---|---|---|---|---|---|---|
| | | | Change | | | Change | | | | |
| | | | from | | | from | | | | |
| Group at | | | baseline | | | baseline | | Pridopidine vs placebo | | |
| 24 weeks | Week | N | (LS means) | SE | N | (LS means) | SE | (LS means) | SE | P Value |
| FAS and | Probable | 13 | 4.79 | 1.0563 | 34 | 0.9 | 0.7384 | −3.89 | 1.3147 | 0.0043 |
| | <24 and slope >=1 | 6 | 5.09 | 2.6572 | 22 | 1.58 | 1.7732 | −3.51 | 3.2513 | 0.2886 |
| | Onset <24 Months | 30 | 3.97 | 0.8906 | 67 | 1.13 | 0.6695 | −2.84 | 1.1204 | 0.0123 |
| | <24 and Svc >60% | 25 | 4.18 | 0.881 | 56 | 1.37 | 0.6457 | −2.81 | 1.096 | 0.0116 |
| | <24 and slope >=0.9 | 8 | 4.55 | 2.1548 | 27 | 1.94 | 1.4155 | −2.61 | 2.6044 | 0.3208 |
| | <18 and slope >=1 | 6 | 3.75 | 2.5468 | 18 | 1.35 | 1.684 | −2.4 | 3.1097 | 0.4477 |
| | Definite and Probable | 41 | 3.88 | 0.7708 | 71 | 1.49 | 0.6297 | −2.39 | 1.004 | 0.0184 |
| | <18 and slope >=0.9 | 7 | 3.54 | 2.2505 | 23 | 1.62 | 1.3937 | −1.92 | 2.694 | 0.481 |
| | Svc >60% | 46 | 3.39 | 0.6406 | 84 | 1.48 | 0.5093 | −1.91 | 0.8205 | 0.0214 |
| | All Patients | 56 | 3.2 | 0.631 | 97 | 1.3 | 0.5282 | −1.9 | 0.8268 | 0.0224 |
| | Onset <18 Months | 20 | 2.45 | 1.1064 | 44 | 1.32 | 0.8005 | −1.13 | 1.3711 | 0.4099 |
| | Definite | 28 | 2.71 | 1.0107 | 37 | 2.34 | 0.946 | −0.37 | 1.416 | 0.7957 |
| | Svc >60% | 33 | 4.34 | 0.757 | 61 | 1.61 | 0.581 | −2.73 | 0.9628 | 0.0054 |
| | <24 and Svc >60% | 16 | 5.06 | 1.1326 | 40 | 1.68 | 0.7704 | −3.38 | 1.3742 | 0.0165 |
| Definite | Definite + Probable | 41 | 3.88 | 0.7708 | 71 | 1.49 | 0.6297 | −2.39 | 1.004 | 0.0184 |
| and | Onset <24 Months | 21 | 4.44 | 1.1285 | 49 | 1.64 | 0.8107 | −2.8 | 1.3986 | 0.0477 |
| probable | <24 and slope >=0.9 | 6 | 6.25 | 2.5442 | 24 | 1.6 | 1.5176 | −4.65 | 3.0187 | 0.1329 |
| and | <24 and slope >=1 | 5 | 6.4 | 2.9731 | 21 | 1.54 | 1.8383 | −4.86 | 3.561 | 0.1818 |
| | <18 and slope >=0.9 | 5 | 5.37 | 2.7412 | 20 | 1.1 | 1.5231 | −4.27 | 3.2092 | 0.1957 |
| | <18 and slope >=1 | 5 | 5.14 | 2.893 | 17 | 1.22 | 1.7552 | −3.92 | 3.4498 | 0.2674 |
| | Onset <18 Months | 13 | 2.98 | 1.627 | 33 | 1.02 | 1.115 | −1.96 | 1.9774 | 0.3269 |
| | Alsfrs-R Pre bl | 9 | 2.77 | 2.1152 | 24 | 1.44 | 1.3991 | −1.33 | 2.6129 | 0.6131 |
| | slp >=0.9 | | | | | | | | | |
| | Alsfrs-R Pre bl | 7 | 2.29 | 2.5351 | 21 | 1.17 | 1.6048 | −1.12 | 3.0632 | 0.7158 |
| | slp >=1 | | | | | | | | | |

### Pridopidine Demonstrates a Significant Beneficial Effect on Speech Characteristics

The effect of pridopidine on speech characteristics was evaluated using Aural Analytics software. Pridopidine demonstrated significant effects on articulation rate (change vs. placebo 0.21±0.085, p=0.0129), speaking rate (change vs, placebo 0.19±0.088, p=0.0277) and phonation time (change vs, placebo 1.37±0.771, p=0.076) as well as a beneficial effect on articulatory precision (change vs, placebo 0.22±0.14, p=0.1138). These effects were further confirmed with the MMRM model in post-hoc analysis (Table 9).

TABLE 9
Pridopidine improves key speech outcome
measure in ALS, all subjects

| Change vs placebo Week 24 | B-spline model | MMRM model |
|---|---|---|
| Articulation Rate (syllables/sec) | 0.21 (0.085) | 0.15 (0.074) |
| | P = 0.0129 | P = 0.047 |
| Speaking Rate (syllables/sec) | 0.19 (0.088) | 0.2 (0.08) |
| | P = 0.0277 | P = 0.009 |
| Phonation Time (Sec) | 1.37 (0.771) | 5.77 (0.9) |
| | P = 0.076 | P = 0.049 |
| Articulatory Precision (ratio) | 0.22 (0.140) | 0.21 (0.08) |
| | P = 0.1138 | P = 0.096 |

Data are LS Means (SE)

Pridopidine has a significant beneficial effect on speaking rate (syllables/second) at 24 weeks. This effect was significant in all subjects, and greatest in subjects with pre-baseline slope ≥0.75 (FIG. 33). In definite+probable ALS subjects, the effect was larger and more significant, especially in subjects <18 months from symptom onset and pre-baseline slope ≥1 (change vs. placebo 1.08, p=0.0003) (FIG. 34).

Pridopidine demonstrated significant improvement in articulation rate as well. As with speaking rate, the effect is largest and most significant in subjects with pre-baseline slope ≥0.75 (change vs. placebo 0.57, p=0.0002) (FIG. 35). Pridopidine's effect on articulation rate was more pronounced in definite+probable ALS subjects, where the effect was most pronounced in subjects <18 months from symptom onset and pre-baseline slope ≥1 (change vs. placebo 1.03, p=0.00002) (FIG. 36).

The effect of pridopidine was also assessed on the fluid biomarker neurofilament light chain (NfL). Serum NfL levels were an exploratory endpoint. Increased biofluid NfL levels are associated with disease progression in ALS. Thus, a decrease in NfL levels can indicate therapeutic efficacy. NfL levels were log-transformed and change from baseline in geometric LS means was calculated.

Pridopidine demonstrated a trend towards reducing NfL levels compared to placebo in the FAS (−4%, p=0.59). This effect was larger in subjects <18 months from symptom onset (−7%, p=0.65) and in subjects with a pre-baseline slope ≥0.75 (−16%, p=0.04) (FIGS. 37 and 38).

The stabilizing effect of pridopidine on NfL levels was more pronounced in definite+probable ALS subjects. The effect was largest in definite+probable subjects <18 months from symptom onset with a pre-baseline slope ≥1, where placebo increased NfL by 8% compared to a decrease of 35% in the pridopidine group (FIG. 39). FIG. 40 illustrates the change vs. placebo in definite+probable subjects, in which pridopidine demonstrates a beneficial effect.

The association between serum NfL levels and changes in ALSFRS-R were evaluated in the FAS <18 months from symptom onset and pre-baseline slope ≥1. In the placebo group (n=17), there was a significant negative association between NfL levels and ALSFRS-R (slope=−3.06±1.4, p=0.043), indicating that higher NfL levels are correlated with disease progression. In the pridopidine group (n=21), the slope is flattened (slope −0.17±1.7, p=0.92) indicating both less worsening and a reduction in NfL levels (FIG. 41A).

The association between serum NfL levels and ALSFRS-R was further confirmed in definite+probable ALS subjects <18 months from symptom onset and pre-baseline slope ≥1. In the placebo group (n=14), NfL levels were significantly, negatively associated with ALSFRS-R score (slope=−3.25±1.6, p=0.046). Pridopidine again demonstrated a stabilizing effect on the association between NfL levels and changes in ALSFRS-R (slope=0.61±1.8, p=0.74). (FIG. 41B).

TABLE 10
A composition combining Pridopidine and Edaravone demonstrates
a greater beneficial effect on ALSFRS-R Total compere to Placebo
group in ALS subjects. (positive change indicates improvement)

| | Edaravone Yes | | Edaravone No | |
|---|---|---|---|---|
| | placebo | pridopidine | placebo | pridopidine |
| n | 41 | 28 | 123 | 92 |

| | | |
|---|---|---|
| Change vs placebo | 0.2 | 0.02 |
| in 24 weeks | | |

The effect of pridopidine on function was assessed using the commonly used ALSFRS-R scale. Pridopidine showed a beneficial effect at 24 weeks in all ALS subjects. This effect was even greater in a composition combining Pridopidine and Edaravone. (Change vs. placebo 0.02, 0.2 respectively) (Table 10).

### Experiment 10

### Pridopidine Improves Survival in ALS

“Survival in ALS clinical trials” refer to the length of time a patient lives without reaching Death or permanent assisted ventilation (PAV)

“Survival probability” refers to the likelihood that a patient will survive for a specific period after receiving a particular treatment or being diagnosed with the disease.

“Survival data” is often presented using Kaplan-Meier curves in the context of clinical trials. These curves illustrate the probability of survival over time and are commonly used in studies where the outcome of interest is time to a specific event, such as death.

“The median survival” (The time to 50% survival probability), indicates the time at which 50% of the participants have died.

“The log-rank test” is used in survival analysis to compare the survival curves of two or more groups.

Pridopidine Showed Clinically Meaningful Impact on Survival in ALS Patients from the HEALY Phase 2 Clinical Trial.

The results are based on the study described in Experiment 9.

Survival analysis was conducted using Kaplan-Meier product-limit estimates, providing a comprehensive view of survival probabilities along with 95% confidence bounds for each treatment group. To assess the overall survival differences between treatment groups, the log-rank test was employed.

In the survival analysis results from Definite, Probable and Early (<18 months since symptom onset) patients showed clinically meaningful survival benefit in favor of pridopidine active group compared to placebo-to-pridopidine group, starting from ˜6 months (180 days), (log rank test: p=0.069)

At approximately 300 days (˜10 months) post first dose, pridopidine active group showed a survival probability of ˜85% compared to ˜50% survival probability for placebo-to-pridopidine group. Median was substantially prolonged by ˜10 months (˜300 to 600 days) in the pridopidine active group compared to the placebo-to-pridopidine group, reflecting an approximate doubling of survival time in this subgroup when treated with pridopidine (FIG. 42).

Pridopidine has a Clinically Meaningful Impact on Survival in ALS Patients Compared to Matched ALS Patients from PRO-ACT Dataset

The impact of pridopidine on survival in ALS patients was further assessed by comparing data from the ALS platform trial with data from the large PRO-ACT database (Pooled Resource Open-Access ALS Clinical Trials). The PRO-ACT database serves as a reliable source of well-matched historical controls for ALS trials. It includes data from 16 ALS clinical trials, 1 large observation study, and over 8 million de-identified longitudinally collected data points from over 8,600 people with ALS. This analysis provides valuable insights into the potential benefits of pridopidine beyond the specific trial context, emphasizing its relevance for a wider spectrum of ALS patients. Analysis was done on PRO-ACT dataset with available survival data.

Propensity score matching was used at baseline to ensure balanced groups, based on key covariates including Time since ALS symptom onset (months), Age at baseline (years), Pre-baseline ALSFRS-R Slope (48-ALSFRS-R at baseline)/Time Since Onset, VC % Predicted Baseline Value (SVC or FVC), El Escorial Criteria (Definite or Probable vs. Other) and Site of Onset (Bulbar vs. Other). This baseline matching criteria ensures balance in covariates between data sets.

The Pridopidine active group (n=120) received pridopidine during the 24-week double-blind and continued to receive pridopidine during OLE (Open Label Extension). PRO-ACT Matched Active group (n=117, with survival data) were matched at baseline with the mentioned covariates to the Pridopidine Active group (Table 11 and Table 12). Placebo-to-Pridopidine group (n=42) received placebo during the 24-week double-blind period and received pridopidine during OLE (“delayed start”). PRO-ACT Matched Placebo group (n=40, with survival data) matched at baseline to the “Placebo-to-Pridopidine” group with the mentioned covariates above. (Table 11 and Table 12).

TABLE 11
Analysis sets matched at baseline:

| | | | |
|---|---|---|---|
| Group 1 | Pridopidine | N = | Received pridopidine during the 24-week |
| | Active | 120 | double-blind. Received pridopidine |
| | | | during OLE |
| Group 2 | PRO-ACT | N = | Matched at baseline to the “Pridopidine |
| | Matched | 117 | Active” group |
| | Active | | |
| Group 3 | Placebo-to- | N = | Received placebo during the 24-week |
| | Pridopidine | 42 | double-blind. Received pridopidine during |
| | | | OLE (“delayed start”) |
| Group 4 | PRO-ACT | N = | Matched at baseline to the “Placebo-to- |
| | Matched | 40 | Pridopidine” group |
| | Placebo | | |

TABLE 12
Well Balanced Baseline Demographics between
groups after propensity score matching

| | | | PRO-ACT | | | | PRO-ACT | |
|---|---|---|---|---|---|---|---|---|
| Baseline | Pridopidine | | matched | | Placebo to | | Matched | |
| characteristics | Active | | Active | | Pridopidine | | Placebo | |
| n | 120 | | 117 | | 42 | | 40 | |
| Age (years) | 58.54 | (10.1) | 60.1 | (9.49) | 57.68 | (9.9) | 56.62 | (9.06) |
| Time since symptom | 20.95 | (8.23) | 20.01 | (9) | 21.82 | (9.98 | 20.31 | (9.62 |
| onset (months) | | | | | | | | |
| ALSFRSR-R | 0.75 | (0.49) | 0.8 | (0.73) | 0.69 | (0.44) | 0.71 | (0.54) |
| pre-baseline slope | | | | | | | | |
| SCV/FVC (%) | 81 | (0.18) | 80 | (0.13) | 80 | (0.17) | 77 | (0.13) |
| Definite or | 75 | (0.43) | 79 | (0.41) | 71 | (0.46) | 80 | (0.41) |
| probable ALS (%) | | | | | | | | |
| Bulbar onset (%) | 19 | (0.4) | 21 | (0.41) | 24 | (0.43) | 32 | (0.47) |

Kaplan-Meier curves showed clinically meaningful and significant survival benefit, between all groups (FIG. 43):
1. Pridopidine Active group vs. PRO-ACT matched Active group log-rank test p<0.001.
   Median survival was ˜23 months in the pridopidine active group vs ˜13 months for the PRO-ACT matched Active group (˜10 months prolongation of survival).
2. Placebo-to-pridopidine group vs. PRO-ACT matched Active group, log-rank p=0.00.4
   Median survival was ˜21 months in the Placebo-to-pridopidine group vs ˜13 months for the PRO-ACT matched Active group (˜8 months prolongation of survival).
3. Pridopidine Active group vs. PRO-ACT matching placebo to pridopidine group, log-rank P=0.005.
   Median survival was ˜23 months in the pridopidine active group vs ˜12 months for the PRO-ACT matched Placebo group (˜11 months prolongation of survival).
4. Placebo-to-Pridopidine group vs. PRO-ACT matching placebo to pridopidine group log-rank p=0.081.
   Median survival was ˜21 months in the Placebo-to-pridopidine group vs. ˜12 months for the PRO-ACT matched Placebo group (˜9 months prolongation of survival)

### Pridopidine Showed Significantly Improved Survival Compared to Matched PRO-ACT Subjects, Using Cox Proportional Hazard Ratio Analysis.

In a comprehensive analysis utilizing the Cox Proportional Hazard Ratio model with a matched active set, pridopidine demonstrated remarkable improvements in survival when compared to matched subjects from the PRO-ACT dataset. The hazard ratio for the pridopidine active group versus the PRO-ACT matched active group was 0.416. This signifies a substantial ˜58% decrease in the risk of death (p<0.001).

Furthermore, placebo-to-pridopidine group in comparison to the PRO-ACT matched active subjects have hazard ratio of 0.466, which translates to ˜53% decrease in the risk of death (p=0.004) (Table 13).

In the analysis utilizing Cox Proportional Hazard Ratio model with a matched placebo set from PRO-ACT dataset, pridopidine showed consistent robust and meaningful survival benefits. The hazard ratio for the pridopidine active group versus the PRO-ACT matched placebo group was 0.0495, indicating ˜50% decrease in the risk of death (p=0.009). In the placebo-to pridopidine group compared to PRO-ACT matched placebo subjects, the hazard ratio was 0.552, indicating ˜45% decrease in the risk of death (p=0.065) (Table 14).

These data support consistent robust and meaningful survival benefit of pridopidine.

TABLE 13
Pridopidine shows significantly improved Survival
compared to matched PRO-ACT-active group subjects,
using Cox Proportional Hazard Ratio Analysis.

| | Hazard ratio | Survival | |
|---|---|---|---|
| | (95% CI) | benefit | P value |
| pridopidine active group vs the | 0.416 | ~58% | <0.001 |
| PRO-ACT matched active group | (0.286, 0.605) | | |
| placebo to pridopidine vs | 0.466 | ~53% | 0.004 |
| PRO-ACT matched active group | (0.279, 0.780) | | |

TABLE 14
Pridopidine shows significantly improved Survival
compared to matched PRO-ACT-placebo group subjects,
using Cox Proportional Hazard Ratio Analysis.

| | Hazard ratio | Survival | |
|---|---|---|---|
| | (95% CI) | benefit | P value |
| pridopidine active group vs the | 0.459 | ~50% | 0.009 |
| PRO-ACT matched placebo group | (0.293, 0.836) | | |
| placebo to pridopidine vs | 0.552 | ~45% | 0.065 |
| PRO-ACT matched placebo group | (0.294, 1.039) | | |

### Pridopidine has a Clinically Meaningful Impact on Survival in Definite, Probable and Early Subgroup of ALS Patients Compared to Treatment Naive PRO-ACT Patients. (FIG. 44)

In the survival analysis results from Definite, Probable and Early Subgroup Kaplan-Meier curves showed clinically meaningful and significant survival benefit, between the following groups:
1. Pridopidine Active group vs. PRO-ACT matched Active group log-rank test p=0.119.
   Median survival was ˜21 months in the pridopidine active group vs ˜15 months for the PRO-ACT matched Active group (˜7 months prolongation of survival).
2. Pridopidine Active group vs. PRO-ACT matching placebo group, log-rank p=0.039.
   Median survival was ˜21 months in the pridopidine active group vs ˜9 months for the PRO-ACT matched Placebo group (˜12 months prolongation of survival).

TABLE 15
Analysis sets matched at baseline in Definite,
Probable and Early Subgroup of ALS patients.

| | | | |
|---|---|---|---|
| Group 1 | Pridopidine | N = | Received pridopidine during the 24-week |
| | Active | 36 | double-blind. Received pridopidine |
| | | | during OLE |
| Group 2 | PRO-ACT | N = | Matched at baseline to the “Pridopidine |
| | Matched | 35 | Active” group |
| | Active | | |
| Group 3 | PRO-ACT | N = | Matched at baseline to the “Placebo-to- |
| | Matched | 10 | Pridopidine” group |
| | Placebo | | |

TABLE 16
PRO-ACT vs pridopidine after matching well balanced at baseline
in Definite, Probable and Early Subgroup of ALS patients.

| | | | PRO-ACT | | PRO-ACT | |
|---|---|---|---|---|---|---|
| Baseline | Pridopidine | | matched | | Matched | |
| characteristics | Active | | Active | | Placebo | |
| n | 36 | | 35 | | 10 | |
| Age (years) | 58.23 | (10.49) | 59.81 | (10.54) | 54.61 | (7.57) |
| Time since symptom | 11.06 | (4.3) | 12.38 | (2.89) | 11.6 | (4.23) |
| onset (months) | | | | | | |
| ALSFRSR-R pre- | 1.31 | (0.73) | 1.1 | (0.66) | 1.1 | (0.89) |
| baseline slope | | | | | | |
| SCV/FVC (%) | 0.8 | (0.48) | 0.8 | (0.17) | 0.79 | (0.13) |
| Definite or probable | 1 | (0) | 1 | (0) | 1 | (0) |
| ALS (%) | | | | | | |
| Bulbar onset (%) | 0.34 | (0.48) | 0.33 | (0.49) | 0.2 | (0.42) |

### Pridopidine in Definite, Probable and Early Subgroup of ALS Patients Shows Significantly Improved Survival Compared to Matched PRO-ACT Subjects, Using Cox Proportional Hazard Ratio Analysis.

In a targeted survival analysis focusing on the Definite, Probable, and Early Subgroup of ALS patients, pridopidine demonstrates a significant improvement in survival outcomes compared to matched PRO-ACT subjects, utilizing Cox Proportional Hazard regression Analysis.

The hazard ratio for the pridopidine active group compared to the PRO-ACT matched active group was 0.588, This signifies a substantial ˜41% decrease in the risk of death.

These data support consistent robust and meaningful survival benefit of pridopidine.

TABLE 17
Pridopidine showed significantly improved Survival
compared to matched PRO-ACT subjects, using
Cox Proportional Hazard Ratio Analysis.

| | Hazard ratio (95% CI) | Survival benefit |
|---|---|---|
| pridopidine active | 0.588 (0.292, 1.182) | 41% |
| group vs the PRO-ACT | | |
| matched active group | | |

### Experiment 11

### Phase 3 Study to Evaluate Efficacy and Safety in ALS (PREVAILS)

A Phase 3, Randomized, Double-blind, Placebo-controlled (DBPC) Study is designed to Evaluate the Efficacy and Safety of Pridopidine in Participants with Amyotrophic Lateral Sclerosis is conducted (PREVAILS).

This Phase 3 study aims to further evaluate the effect of pridopidine 45 mg b.i.d on disease progression, survival, speech, bulbar functions, respiratory function, pseudobulbar affect, biomarkers, quality of life as well as to further characterize the safety profile in participants with ALS.

Table 18 provides the Objectives and Endpoints of this study:

TABLE 18
Objectives and Endpoints (Primary, Secondary and Safety)

| | |
|---|---|
| Objectives | Endpoints |

Primary

| | |
|---|---|
| To evaluate the effect of pridopidine | Change from baseline through Week 26 in the |
| on disease progression in participants | ALSFRS-R total score adjusted for mortality |
| with ALS | Change from baseline through Week 48 in the |
| | ALSFRS-R total score adjusted for mortality |

Secondary

| | |
|---|---|
| To evaluate the effect of pridopidine | Overall survival at Week 96 (end of OLE) |
| on survival | |
| To evaluate the impact of pridopidine | Change from baseline through Week 26, |
| on speech | Week 48 in: |
| | Speaking rate as measured by quantitative |
| | speech assessment in the clinic |
| | Intelligibility of speech as measured by |
| | quantitative speech assessment in the |
| | clinic |
| To evaluate the effect of pridopidine | Change from baseline through Week 48 in |
| on the decline in Respiratory function | slow vital capacity (SVC) |
| To evaluate the effect of pridopidine | Change from baseline through Week 48 in the |
| on the decline in Bulbar function | bulbar subdomain of the ALSFRS-R |
| To evaluate the effect of pridopidine | Change from baseline at Week 48 in the |
| on Quality of Life | ALSAQ-40 |
| To evaluate the long-term effect of | Change from baseline in the ALSFRS-R total |
| pridopidine on various functional | score adjusted for mortality through Week 96 |
| parameters in participants with ALS | (end of Open label extension-(OLE)) |
| To evaluate the long-term effect of | Change from baseline through Week 96 (end |
| pridopidine on additional efficacy | of OLE) in: |
| measures of disease progression, | Speaking rate and intelligibility of speech |
| quality of life, and respiratory function | as measured by quantitative speech |
| | assessment in the clinic |
| | SVC (Slow vital capacity) |
| | ALSFRS-R bulbar subdomain |
| | Quality of Life (ALSAQ-40) |

Safety

| | |
|---|---|
| To evaluate the safety and tolerability | Incidence and severity of treatment emergent |
| of pridopidine in participants with | adverse events (TEAEs), adverse events of |
| ALS | special interest (AESIs), and serious adverse |
| | events (SAEs) in DBPC and OLE |
| | Incidence and shifts of clinically significant |
| | abnormalities in electrocardiogram (ECG), |
| | laboratory tests, and vital signs in DBPC and |
| | OLE |
| | Change from baseline in Columbia-Suicide |
| | Severity Rating Scale (C-SSRS) in DBPC and |
| | OLE |
| | Tolerability in DBPC and OLE: |
| | The number (%) of participants who |
| | complete the Treatment period |
| | The number (%) of participants who fail |
| | to complete the Treatment period due to |
| | TEAEs |
| | The number (%) of participants who fail |
| | to complete the Treatment period due to |
| | stopping rules |

Exploratory

| | |
|---|---|
| To evaluate the effect of pridopidine | Change from baseline through Week 96 (end |
| on patient reported outcomes (PROs) | of OLE) in: |
| | Communication as measured by The |
| | Communicative Participation Item Bank |
| | (CPIB) |
| | European Quality of Life-5-Dimension |
| | (EQ-5D) including the EQ visual analog |
| | scale (EQ VAS) |
| | ALSFRS-R subdomains as ranked by the |
| | Patient-Ranked Order of Function |
| | (PROOF) |
| To evaluate the effect of pridopidine | Change from baseline through Week 96 (end |
| on other efficacy measures | of OLE) in: |
| | Center for Neurologic Study Lability |
| | Scale (CNS-LS) |
| | Additional exploratory quantitative |
| | speech assessments (eg, naturalness) |
| Pharmacokinetics and Biomarkers | |
| To evaluate the pridopidine exposure | Plasma concentration of pridopidine and |
| in participants with ALS | metabolite in DBPC and OLE |
| To evaluate changes in disease | Change from baseline in biomarkers of |
| biomarkers in serum | neurodegeneration (eg, neurofilament light |
| | [NfL], glial fibrillary acidic protein [GFAP], |
| | YKL-40) in DBPC and OLE |

This is a Phase 3, randomized study consisting of a double-blind placebo-controlled (DBPC) period followed by an open label extension (OLE) to evaluate the efficacy and safety of pridopidine administered orally at a dose of 45 mg bid in participants with early and rapidly progressing ALS. Standard of care treatments will be allowed as long as participants are on stable dose for at least 4 weeks prior to dosing.

In the DBPC period, participants are randomized in a 3:2 ratio to one of two arms: active arm (pridopidine) or control arm (placebo); randomization is stratified at Day 1 by the presence of Bulbar involvement, defined as ALSFRS-R bulbar domain score <12 (yes/no), active use of dextromethorphan hydrobromide and quinidine sulfate (DM/Q; Nuedexta®) or its components (yes/no), and active use of edaravone (Radicava®) (yes/no).

In the OLE, all participants receive pridopidine through Week 96, while maintaining the blind to their original randomization for both the participant as well as the Investigator. Participants who complete the 48-week DBPC period enter into the OLE, starting at the Week 48 visit, and receive open-label pridopidine. The OLE consist of a 48-week treatment period, including an initial two-week (re) titration period for all participants at pridopidine 45 mg qd (as described for the DBPC) and a maintenance period of 46 weeks with pridopidine 45 mg bid. The original randomization is maintained blinded for all participants, investigators, site staff and CRO until the end of the OLE.

All participants follow for safety for two weeks after the last dose of study drug. For each participant, the last visit will be the End of Study (EoS) visit scheduled at Week 98 if the participant completes all study visits, or the Early Termination (ET) visit if the participant withdraws from study before Week 96 (End of Treatment).

### Study Duration:

For each participant, the study is expected to last as follows:

TABLE 19

| Study Period | Duration |
|---|---|
| Screening Period | Up to 4 weeks |
| Treatment Period | 96 weeks |
| DBPC | 48 weeks (two weeks titration + 46-week maintenance) |
| OLE | 48 weeks (two weeks titration + 46-week maintenance) |
| Follow-up Period | 2 weeks |
| Total Study Duration | Up to 102 weeks* |

Abbreviations: DBPC, double-blind, placebo-controlled; OLE, open-label extension
*Survival assessment will continue until End of Study.

The study comprises a screening period (up to 4 weeks), two treatment periods, the: DBPC and OLE (each consisting of 48 weeks), and a follow-up period (two weeks after the last dose).

### Drug Administration

| ARM Name | Active | Control |
|---|---|---|
| Study Part | DBPC and OLE | DBPC only |
| Study Drug Name | Pridopidine | Placebo |
| (INN) | | |
| Type | Drug (small molecule) | — |
| Dose Formulation | Hard gelatin capsules | Hard gelatin capsules |
| Unit Dose Strength(s) | 45 mg | NA |
| Dosage Level(s) | | |
| Titration period | 45 mg capsule qd (morning | Placebo capsule qd (morning |
| | dose) for 2 weeks ± 2 days | dose) for 2 weeks ± 2 days |
| Main full-dose | 45 mg capsule bid (1 capsule in | Placebo capsule bid (1 capsule |
| Treatment period | the morning and 1 capsule in the | in the morning and 1 capsule in the |
| | afternoon, 7 to 10 hours after | afternoon, 7 to 10 hours after |
| | morning dose and before 7:00 | morning dose and before 7:00 |
| | pm); total daily dose of 90 mg | pm) |
| Route of | Oral | Oral |
| Administration | | |

### Efficacy Assessment

### ALS Functional Rating Scale-Revised (ALSFRS-R)

The ALSFRS-R is the most widely used instrument for assessing functional deficits in ALS. The scale is disease-specific and encompasses 12 questions grouped into four domains to assess bulbar symptoms, limb and trunk functionality, respiratory symptoms, and the need for percutaneous endoscopic gastrostomy, non-invasive ventilation, or tracheostomy with invasive ventilation. Each function is scored from 0 (no ability) to 4 (normal), with a minimum total score of 0 and maximum total score of 48 (Cedarbaum, 1999; Bakker, 2017). Higher scores indicate better functioning. To the extent possible, there should be a single rater for a participant throughout the study visits.

### Speech Assessment

Changes in Speaking Rate, as assessed either by words per minute or syllables per second, is an early and sensitive measure of speech impairment in ALS not only in those with bulbar onset, but also in those with limb-onset as the disease inevitably spreads and the bulbar system becomes involved/impaired. Speaking Rate is a preferred assessment for early changes in speech because it is readily measurable, robust as to acquisition conditions (timing measures can be reliably acquired on lower quality devices [MOU1]), has high intra- and inter-rater reliability (ICC of 0.99-100), and perhaps most importantly, declines earlier in the disease than either the speech question of the ALSFRS-R or measures like intelligibility (Ball, 2002; Green, 2013; Stipancic, 2018).

Composite (naturalness/intelligibility) and motor features of speech (speaking rate) are measured by quantitative speech assessment in the clinic using vendor provided equipment. Motor speech function is recorded via an automated algorithmic digital application running on a tablet supplied by the vendor. The speech assessment is conducted in a standardized manner during clinic visits with site staff guiding participants through the task battery, such as producing a sustained vowel, repeating syllables and reading a short passage. The overall duration of the speech battery is approximately 10 minutes. Task prompts appear in the respective local language.

The recordings are uploaded to the vendor portal and are checked for quality and completeness prior to analysis, which includes metrics across motor domains of speech, voice, and breath support that relate to ALS.

### Respiratory Function—Slow Vital Capacity (SVC)

SVC is assessed in the sitting position with a spirometer. SVC measures the maximal amount of air exhaled in a relaxed expiration from full inspiration to residual volume; exhalation should be terminated after 15 seconds (Pinto, 2019).

### Survival Assessment

Survival assessment is defined as date of death or date last known alive or death equivalent (tracheostomy or PAV, defined as use of mechanical ventilation for more than 22 hours a day for more than 7 consecutive days) for each randomized participant at the time of Week 48, Week 96 (or ET visit for participants that discontinue study treatment), and at the End of Study timepoint.

### Patient Reported Outcomes (PROs)

ALSFRS-R Patient-ranked Order of Function (PROOF) The ALSFRS-R PROOF endpoint builds on the ALSFRS-R by adding the additional patient reported questions: ‘Which domain bothers you the most?’ and ‘Imagine you will receive a treatment that delays disease progression; delay of which domain is the most important to you?’. The latter can be answered by ranking the four domains in their order of importance using ranks 1 (most important) to 4 (least important), or by indicating that there is no preference (ie all domains are of equal importance). These additional questions are used to prioritize the four ALSFRS-R subdomain scores based on the participant-reported impact and importance. This method ensures a comprehensive and participant-centered evaluation of the treatment effect, capturing the aspects of the disease that matter most to participants (van Eijk, 2022b).

Amyotrophic Lateral Sclerosis Assessment Questionnaire—40 (ALSAQ-40) ALSAQ-40 includes 40 items/questions with 5 discrete scales including physical mobility (10 items), activities of daily living and independence (10 items), eating and drinking (3 items), communication (7 items), and emotional reactions (10 items). The questions refer to two weeks prior to the day of the application and the answers are given according to the following variables: never, rarely, sometimes, frequently and always. The score ranges from 0 to 100; the higher the score, the worse the quality of life (Jenkinson, 1999a; Jenkinson, 1999b; Jenkinson, 2000).

European Quality of Life-5-Dimension-5 Level (EQ-5D-5L) The EQ-5D-5L consists of the EQ-5D descriptive system and the EQ visual analogue scale (EQ VAS) (Herdman, 2011). The EQ-5D-5L descriptive system evaluates an individual's health across five dimensions: mobility, self-care, usual activities, pain/discomfort, and anxiety/depression, with each dimension having multiple severity levels. Each dimension has 5 levels: no problems, slight problems, moderate problems, severe problems, and extreme problems. The participant is asked to indicate his/her health state by ticking the box next to the most appropriate statement in each of the five dimensions. This decision results in a 1-digit number that expresses the level selected for that dimension. The digits for the five dimensions can be combined into a 5-digit number that describes the participant's health state. The EQ VAS records the participant's self-rated health on a vertical visual analogue scale where the endpoints are labeled ‘The best health you can imagine’ and ‘The worst health you can imagine’. The VAS is used as a quantitative measure of health outcome that reflects the participant's own judgment.

Center for Neurologic Study-Lability Scale (CNS-LS) The Center for Neurologic Study-Lability Scale (CNS-LS) is a self-administered, seven-item questionnaire designed to measure the frequency of episodes of PBA, which includes involuntary and inappropriate laughing and crying. It is used primarily in participants with neurological conditions such as ALS and multiple sclerosis (MS). The CNS-LS helps in evaluating the severity of affective lability by asking participants to rate their experiences over the past week. Each item is scored on a scale from 1 (applies never) to 5 (applies most of the time), with a total score ranging from 7 to 35. A score of 13 or higher may suggest the presence of PBA, although it is not a definitive diagnostic tool. The CNS-LS has been validated in ALS and MS participant populations, showing good test-retest reliability and internal consistency (Moore, 1997). To be conducted in English-speaking countries and countries with an official translation only.

Communicative Participation Item Bank (CPIB) The CPIB provides a unidimensional, self-report outcome measurement instrument dedicated to the construct of communicative participation. The CPIB was developed with the intent of being appropriate for community-dwelling adults in the variety of conversational situations they frequently engage in as part of life roles at home, at work, and in social and leisure situations. All items in the CPIB start with the stem, ‘Does your condition interfere with . . . ’ followed by various conversational situations such as ‘Making a phone call to get information,’ or ‘Having a conversation in a noisy place.’ Respondents choose from four response categories to rate the level of interference they experience in that situation, ranging from 0 (Not at all) to 3 (Very much).′ The CPIB reflects the degree to which ALS disease-related changes in speech influence.

### Inclusion Criteria

Participants are enrolled to the study if any of the following criteria apply:
a. Adult participants (age 18-80, inclusive) with a diagnosis of Definite ALS or Probable ALS using the El Escorial criteria, within 18 months of symptom onset and a Treatment Initiative to Cure ALS (TRICALS) Risk Profile Calculator score in the range of −6< to <−2.
b. Definite ALS or Probable ALS using the El Escorial criteria (Brooks, 2000).
c. Symptom onset* of ≤18 months at screening.
* The date of onset is the date the participant reported one or more of the following symptoms: Muscle weakness, Speech/swallowing difficulties, Respiratory symptoms, Slow Vital Capacity (SVC) greater or equal to 60% according to the GLI-2012 reference standard. (GLI-2012)
d. TRICALS Risk Score, based on the European Network for the Cure of ALS (ENCALS) survival prediction model (Westeneng, 2018; van Eijk, 2019), >−6 and <−2 at screening
e. Contraceptive use should be consistent with local regulations regarding the methods of contraception for those participating in clinical studies.
f. Individuals of childbearing potential (IOCBP) must have a negative β-human chorionic gonadotropin (β-hCG) test at screening (serum) and Day 1 (urine).
g. IOCBP who are sexually active with male partners of reproductive potential (ie, without vasectomy) must use double contraception (including a barrier contraceptive and another highly effective method) for at least 3 months prior to Screening, for the duration of the study, and for 30 days after last dose of study drug. Participants must also refrain from oocyte donation for the purpose of reproduction during this period. Exceptions are made for surgically sterile participants, or post-menopausal participants (defined as 12 months of spontaneous amenorrhea or 6 months of spontaneous amenorrhea with serum follicle stimulating hormone levels >40 mIU/mL or 6 weeks postsurgical bilateral oophorectomy with or without hysterectomy).
h. Male participants must be sterile, or if they are potentially fertile/reproductively competent (not surgically [eg, vasectomy] or congenitally sterile) and their individual partners are of childbearing potential, they must use, together with partners of child-bearing potential, effective birth control methods (including a barrier contraceptive and another method) for the duration of the study and for 90 days after last dose of study drug discontinuation. Male participants must refrain from sperm donation throughout the study and for 90 days after last dose of study drug.
i. Able to swallow a capsule at the time of the Day 1 visit.
*The Participants transitioning to enteral feeding such as percutaneous endoscopic gastrostomy (PEG) tube during the course of the study may be allowed to continue the study on study drug.
j. Participants currently taking riluzole and/or edaravone in countries with regulatory approval, may continue to do so provided they have been on a stable dose of riluzole for ≥4 weeks prior to Day 1 and/or a stable dose of edaravone for at least one treatment cycle and plan to remain on this medication regimen for the remainder of the study.

### Exclusion Criteria:

Participants are excluded from the study if any of the following criteria apply:

### Medical Conditions

a. Presence of tracheostomy or permanent assisted ventilation (PAV).
b. Clinically significant heart disease, clinically significant history of arrhythmia, symptomatic or uncontrolled atrial fibrillation despite treatment, or asymptomatic sustained ventricular tachycardia, or presence of left bundle branch block.
c. Presence of unstable psychiatric disease, cognitive impairment, dementia or substance abuse (as defined by the Diagnostic and Statistical Manual-Fifth Edition (DSM-5) Text Revision criteria for substance use) that would impair ability of the participant to provide informed consent and participate in the study, according to Investigator judgment.
d. Clinically significant and/or unstable medical condition (other than ALS) that may either pose a clinically meaningful risk to the participant and/or to study completion, in the opinion of the Investigator, including but not limited to:
   Cardiovascular dysfunction, history of clinically significant electrocardiogram [ECG] findings, uncontrolled hypertension
   Systemic infection (acute or chronic), confirmed Hepatitis B virus [HBV], Hepatitis C virus [HCV], confirmed human immunodeficiency virus [HIV], acquired immune deficiency syndrome
   History of seizure/epilepsy in the previous 5 years
   Unstable psychiatric or other neurologic disorders [eg, additional movement disorders or cognitive impairment not related to ALS such as Alzheimer's disease, Parkinson's disease, or frontotemporal dementia]
   Any history of metastatic cancer, any malignancy requiring active cancer treatment (eg chemotherapy, radiotherapy, immunotherapy, or targeted therapy) within the last 12 months, or any cancer diagnosed within the previous 5 years, unless localized and clearly resolved (eg, basal cell carcinoma, squamous cell carcinoma of the skin, cervical carcinoma in situ, papillary thyroid cancer post-thyroidectomy).
   Additional systemic disease, unstable thyroid dysfunction, unexplained and/or clinically significant laboratory values outside of the reference range
   Known intracranial neoplasms, vascular malformations, history of cerebrovascular event, or intracranial hemorrhage.
e. Active suicidal ideation or intent as measured by a most severe suicide ideation score of 4 (Active Suicidal Ideation with Some Intent to Act, without Specific Plan) or 5 (Active Suicidal Ideation with Specific Plan and Intent) on the C-SSRS if the ideation or intent occurred within one year of Screening, or participants who answered “Yes” on any of the five C-SSRS Suicidal Behavior Items (actual attempt, interrupted attempt, aborted attempt, preparatory acts, or behavior), if the attempt or acts were performed within one year of Screening, or participants who, in the opinion of the Investigator, present a serious risk of suicide.

### Prior Concomitant Therapy

a. Use of Nuedexta® or DM/Q at a dosage higher than 20 mg dextromethorphan+10 mg quinidine bid. Note: In countries with regulatory approval, participants are allowed to be on riluzole and/or edaravone and/or Nuedexta (DM/Q) (at an equivalent or lower dose than stated above) as long as they have been on a stable dose of medication for ≥4 weeks prior to the Day 1 visit and plan to remain on the same dose of this medication for the remainder of the study.
b. Use of medications that prolong QT interval, taken within four weeks prior to Day 1 visit (note, Amiodarone is not allowed within six weeks of Day 1 visit) or at any timepoint during the study.
c. Previous treatment with pridopidine.
d. Any previous treatment with gene therapy or antisense oligonucleotides.
e. Treatment with any investigational product within 6 weeks or 5 half-lives (whichever is longer) before Day 1 or a plan to participate in another clinical study that assesses any investigational product during the study.

### Diagnostic Assessments

a. Abnormal liver function at screening defined as aspartate aminotransferase (AST) and/or alanine aminotransferase (ALT)>three times the upper limit of the normal range (ULN).
b. Total bilirubin >1.5 mg/dL, except participants with unconjugated hyperbilirubinemia without other liver function derangements or other explanations for the elevated bilirubin (eg consistent with diagnosis of Gilbert's syndrome).
c. Severe renal insufficiency at screening as defined by eGFR <30 mL/min/1.73 m².
d. Confirmed prolonged Fridericia-corrected QT (QTcF) interval (defined as a QTcF interval of >450 msec for male participants and >470 msec for female participants).
e. Known history of long QT syndrome or a first degree relative with this condition.
f. Confirmed mutation in the SOD1 or C9orf72 gene.

### Other Exclusion Criteria

a. Implantation of Diaphragm Pacing System (DPS).
b. History of known allergy to pridopidine, silicified microcrystalline cellulose, or magnesium stearate.
c. Pregnancy (confirmed by a pregnancy test within seven days of first dose of study drug for IOCBP) or currently breastfeeding or intending to become pregnant during the study.
d. Vulnerable participant (eg, people kept in detention), or participant unfit to participate in a clinical study due to living circumstances (eg, without sufficient family or social support, stable residence, sustainable financial and general healthcare and resources).
e. Any direct relation to study staff or Sponsor.

### REFERENCES

Bakker, L. A., et al. (2017). “Assessment of the factorial validity and reliability of the ALSFRS-R: a revision of its measurement model.” J Neurol 264(7): 1413-1420.
Ball, L., et al. (2002). “Timing of speech deterioration in people with amyotrophic lateral sclerosis.” J Med Speech Lang Pathol 10(4): 231-235.
Bilsland et al. (2010) Deficits in axonal transport precede ALS symptoms in vivo. Proc Natl Acad Sci USA. 107(47):20523-8.
Bonni A, et al. (1999) Cell survival promoted by the Ras-MAPK signaling pathway by transcription-dependent and -independent mechanisms. Science 286:1358-1362.
Bozzoni et al. (2016) Amyotrophic lateral sclerosis and environmental factors”, Funct. Neurol. 31(1):7-19.
Brooks, B. R. (1994). El Escorial World Federation of Neurology criteria for the diagnosis of amyotrophic lateral sclerosis. Subcommittee on Motor Neuron Diseases/Amyotrophic Lateral Sclerosis of the World Federation of Neurology Research Group on Neuromuscular Diseases and the El Escorial “Clinical limits of amyotrophic lateral sclerosis” workshop contributors. Journal of the neurological sciences, 124, 96-107.
Brooks, B. R., et al. (2000). “El Escorial revisited: revised criteria for the diagnosis of amyotrophic lateral sclerosis.” Amyotroph Lateral Scler Other Motor Neuron Disord 1(5): 293-299.
Cedarbaum, J. M., et al. (1999). “The ALSFRS-R: a revised ALS functional rating scale that incorporates assessments of respiratory function. BDNF ALS Study Group (Phase III).” J Neurol Sci 169(1-2): 13-21.
De Vos et al. (2007) Familial amyotrophic lateral sclerosis-linked SOD1 mutants perturb fast axonal transport to reduce axonal mitochondria content. Hum Mol Genet. 16(22):2720-2728.
Eykens C, Robberecht W (2015) The genetic basis of amyotrophic lateral sclerosis: recent breakthroughs. Adv Genomics Genetics 5:327-345.
Geva et al. (2016). Pridopidine activates neuroprotective pathways impaired in Huntington Disease. HMG 25(18): 3975-87.
Green, J. R., et al. (2013). “Bulbar and speech motor assessment in ALS: challenges and future directions.” Amyotroph Lateral Scler Frontotemporal Degener 14(7-8): 494-500.
Hammarström P, et al, (2010) A fluorescent pentameric thiophene derivative detects in vitro-formed prefibrillar protein aggregates. Biochemistry 49:6838-45.
Herdman, M., et al. (2011). “Development and preliminary testing of the new five-level version of EQ-5D (EQ-5D-5L).” Qual Life Res 20(10): 1727-1736.
Ionescu et al. (2016) Compartmental microfluidic system for studying muscle-neuron communication and neuromuscular junction maintenance. (2016) European Journal of Cell Biology 95(2) 69-88.
Jenkinson, C., et al. (1999a). “Development and validation of a short measure of health status for individuals with amyotrophic lateral sclerosis/motor neurone disease: the ALSAQ-40.” J Neurol 246 Suppl 3: Iii16-21.
Jenkinson, C., et al. (1999b). “Evidence for the validity and reliability of the ALS assessment questionnaire: the ALSAQ-40.” Amyotroph Lateral Scler Other Motor Neuron Disord 1(1): 33-40.
Jenkinson, C., Levvy, G., Fitzpatrick, R. and Garratt, A. (2000). “The amyotrophic lateral sclerosis assessment questionnaire (ALSAQ-40): tests of data quality, score reliability and response rate in a survey of patients.” J Neurol Sci 180(1-2): 94-100.
Langa F et al. (2003) Generation and phenotypic analysis of sigma receptor type I (sigma 1) knockout mice. Eur J Neurosci. 18:2188-96.
Martel et al. (2016) From animal models to human disease: a genetic approach for personalized medicine in ALS, Acta Neuropathol. Commun. 4(1):70.
Millecamps and Julien (2013) Axonal transport deficits and neurodegenerative diseases. Nat Rev Neurosci. 14:161-76.
Moore, S. R., et al. (1997). “A self report measure of affective lability.” J Neurol Neurosurg Psychiatry 63(1): 89-93.
Perlson, et al. (2009) A Switch in Retrograde Signaling from Survival to Stress in Rapid Onset Neurodegeneration. J Neurosci. 2009 29(31): 9903-9917.
Pinto, S. and de Carvalho, M. (2019). “SVC Is a Marker of Respiratory Decline Function, Similar to FVC, in Patients With ALS.” Front Neurol 10:109
Riva et al. (2016) Recent advances in amyotrophic lateral sclerosis, J. Neurol. 263:1241-1254.
Sahlholm et al. (2015) Pridopidine selectively occupies sigma-1 rather than dopamine D2 receptors at behaviorally active doses. Psychopharm. 232(18):3342-53.
Smith, et al. (2017). Enhanced Bulbar Function in Amyotrophic Lateral Sclerosis: The Nuedexta Treatment Trial. Neurotherapeutics, 14(3), 762-772.
Smith, R. A., at el. (2018). Assessment of bulbar function in amyotrophic lateral sclerosis: validation of a self-report scale (Center for Neurologic Study Bulbar Function Scale). European journal of neurology, 25(7), 907-e66.
Spiller, K. J. et al. (2016) Selective motor neuron resistance and recovery in a new inducible mouse model of TDP-43 proteinopathy. J. Neurosci. 36, 7707-7717.
Spiller, K. J. et al. (2018) Microglia-mediated recovery from ALS-relevant motor neuron degeneration in a mouse model of TDP-43 proteinopathy. Nat. Neurosci. 21, 329-340.
Stegmann, G., et al. (2020) Early detection and tracking of bulbar changes in ALS via frequent and remote speech analysis, npj Digit. Med. 3, 132. Song et al. (2013) An update on amine oxidase inhibitors: Multifaceted drugs, Prog. Neuropyschopharmacol. Biol. Psychiatry 44:118-124.
Stipancic, K. L., et al. (2018). “Minimally detectable change and minimal clinically important difference of a decline in sentence intelligibility and speaking rate for individuals with amyotrophic lateral sclerosis.” J Speech Lang Hear Res 61(11): 2757-2771.
van Eijk, R. P. A., et al. (2019). “Refining eligibility criteria for amyotrophic lateral sclerosis clinical trials.” Neurology 92(5): e451-e460.
van Eijk, R. P. A., et al. (2022b). “Composite endpoint for ALS clinical trials based on patient preference: Patient-Ranked Order of Function (PROOF).” J Neurol Neurosurg Psychiatry 93(5): 539-546.
Walker, A. K. et al. Functional recovery in new mouse models of ALS/FTLD after clearance of pathological cytoplasmic TDP-43. Acta Neuropathol. 130, 643-660 (2015).
Westeneng, H.-J., Debray, T. P. A., Visser, A. E., van Eijk, R. P. A., Rooney, J. P. K., Calvo, A., et al. (2018). “Prognosis for patients with amyotrophic lateral sclerosis: development and validation of a personalised prediction model.” The Lancet Neurology 17(5): 423-433.
Zahavi, et al. (2015) A compartmentalized microfluidic neuromuscular co-culture system reveals spatial aspects of GDNF functions. J. Cell Sci. 128, 1241-1252.
Zou et al. (2016) Toward precision medicine in amyotrophic lateral sclerosis, Ann. Transl. Med. 4(2):27.
Riluzole—Drug Summary, PDR (Prescribers' Digital Reference), www.pdr.net/drug-summary/Rilutek-riluzole-526 accessed Jul. 28, 2017
Edaravone—Drug Summary, PDR (Prescribers' Digital Reference), www.pdr.net/drug-summary/Radicava-edaravone-24080 accessed Jul. 28, 2017
Dextromethorphan hydrobromide/quinidine sulfate—Drug Summary, PDR (Prescribers' Digital Reference), http://www.pdr.net/drug-summary/Nuedexta-dextromethorphan-hydrobromide-quinidine-sulfate-1344.3281 accessed Aug. 14, 2017
U.S. Pat. No. 7,923,459
U.S. Pat. No. RE46117
PCT Application Publication No. WO 2017/015609

What is claimed is:

1. A method for maintaining, improving, or lessening the decline of symptoms associated with ALS in a subject in need thereof wherein the symptom is impaired: functionality, respiratory function, bulbar function, speech, muscle strength or any combination thereof, wherein the method comprises administering to the subject a composition comprising a therapeutically acceptable amount of pridopidine or pharmaceutically acceptable salt thereof.

2. The method of claim 1, wherein the symptom is impairment in speech.

3. The method of claim 2, wherein the impairment of speech comprises reduced speaking rate, reduced phonation time, reduced articulation rate, reduced speech intelligibility and reduced articulation precision.

4. The method of claim 1, wherein ALS patient's impaired functionality comprises speech, salivation, swallowing, handwriting, cutting food and handling utensils, dressing and hygiene, turning in bed and adjusting bed clothes, walking, climbing stairs, dyspnea, orthopnea, respiratory insufficiency or any combination thereof.

5. The method of claim 1, wherein impaired respiratory function is assessed by slow vital capacity (SVC) or forced vital capacity (FVC) or by the ALSFRS-R-Respiratory sub-domain.

6. The method of claim 1, wherein maintaining, improving, or lessening the decline in bulbar function is measured by the ALSFRS-R bulbar subdomain (Q1-Q3) score.

7. The method of claim 1, wherein said maintaining, improving, or lessening the decline in bulbar function is measured by the CNS-BFS.

8. The method of claim 1, wherein ALS patient's impaired bulbar function comprises impaired speech, swallowing or salivation.

9. The method of claim 1, wherein the subject has TRICALS Risk Score between >6 and <−2, a disease duration ≤18 months from symptom onset, and a Slow Vital Capacity (SVC) ≥60% of predicted according to GLI-2012 standards.

10. The method of claim 1, wherein the amount of pridopidine or pharmaceutically acceptable salt thereof is effective in maintaining, reducing or lessening the increase in neurofilament light (NfL) protein levels in a human subject afflicted with ALS.

11. The method of claim 1, wherein the amount of pridopidine or pharmaceutically acceptable salt thereof is effective in maintaining, reducing or lessening levels of glial fibrillary acidic protein (GFAP) or YKL-40.

12. The method of claim 1, wherein said subject has faster disease progression as measured by the ALSFRS-R pre-baseline slope.

13. The method of claim 1, wherein said subject has faster disease progression as measured by the baseline NfL levels, GFAP, YKL-40 or combination thereof.

14. The method of claim 1, wherein the method further comprises assessing a biomarker associated with ALS progression or treatment response: wherein the biomarker comprises cardiac Troponin T (cTnT), and wherein the subject has elevated cTnT levels at baseline, demonstrates a longitudinal change in cTnT levels during treatment, or is stratified according to cTnT levels for assessing disease severity or progression rate.

15. The method of claim 1, wherein said subject has early ALS with less than 18 months from symptom onset.

16. The method of claim 1, wherein said subject has faster disease progression as measured by the ALSFRS-R pre-baseline slope and early with <18 months from symptom onset.

17. The method of claim 1, wherein the maintaining, improving, or lessening the decline is measured by the ALS Functional Rating Scale-Revised (ALSFRS-R).

18. The method of claim 1, wherein the treatment efficacy is measured by ALSFRS-R Patient-Ranked Order of Function (PROOF), the ALS Assessment Questionnaire-40 (ALSAQ-40), the Communicative Participation Item Bank (CPIB), the EQ-5D-5L including EQ-VAS or combination thereof.

19. The method of claim 1, wherein survival is further evaluated through Week 96, with death equivalents defined as tracheostomy or permanent assisted ventilation (>22 hours/day for >7 consecutive days).

20. The method of claim 1, wherein the amount of pridopidine or pharmaceutically acceptable salt thereof is administered daily, twice a week, three times a week or more often than once daily.

21. The method of claim 1, wherein the amount of pridopidine or pharmaceutically acceptable salt thereof is administered orally.

22. The method of claim 1, wherein the amount of pridopidine or pharmaceutically acceptable salt thereof administered is 10 mg per day to 90 mg per day.

23. The method of claim 1, wherein the pridopidine salt is pridopidine hydrochloride.

24. The method of claim 1, further comprising administering a second composition to the subject comprising a therapeutically effective amount of a Second compound, wherein the Second compound is riluzole, edaravone, dextromethorphan/quinidine, sodium phenylbutyrate (PB), tauroursodeoxycholic acid, sodium phenylbutyrate (PB)/tauroursodeoxycholic acid, SLS-005 (Trehalose), DNL343, CNM-Au8 nanocrystalline gold or ABBV-CLS-7262.

25. The method of claim 23, wherein the administration of the Second compound precedes the administration of pridopidine or pharmaceutically acceptable salt thereof.

26. The method of claim 23, wherein the administration of pridopidine or pharmaceutically acceptable salt thereof precedes the administration of the Second compound.

27. The method of claim 23, wherein the pridopidine or pharmaceutically acceptable salt thereof, is administered adjunctively to the Second compound.

28. The method of claim 23, wherein the Second compound is administered adjunctively to the pridopidine or pharmaceutically acceptable salt thereof.
